(19) United States
(12) Patent Application Publication
(10) Pub. No.: US 2004/0249144 A1
(43) Pub. Date: Dec. 9, 2004

(54) Regulated breast cancer genes

(76) Inventors: Zairen Sun, Rockville, MD (US); Xuan Li, Silver Spring, MD (US); Gilbert Jay, North Bethesda, MD (US); Karl F. Kovacs, Rockville, MD (US); Wufang Fan, Germantown, MD (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/491,566

(22) PCT Filed: Oct. 2, 2002
(86) PCT No.: PCT/US02/31287

Related U.S. Application Data
(63) Continuation-in-part of application No. 10/144,194, filed on May 14, 2002.
(60) Provisional application No. 60/326,526, filed on Oct. 3, 2001.

(51) Int. Cl. C12Q 1/68; C07H 21/04; C07K 14/47

(52) U.S. Cl. 536/23.5; 435/6; 435/69.1; 435/320.1; 435/325; 530/350

(57) ABSTRACT

The present invention relates to all facets of novel polynucleotides, the polypeptides they encode, antibodies and specific binding partners thereto, and their applications to research, diagnosis, drug discovery, therapy, clinical medicine, forensic/science and medicine, etc. The polynucleotides are differentially-regulated in breast cancer and are therefore useful in variety of ways, including, but not limited to, as molecular markers, as drug targets, and for detecting, diagnosing, staging, monitoring, predisposition to, etc., diseases and conditions relating to breast cancer.

This application claims the benefit of U.S. Provisional Application Ser. No. 60/326,526, filed Oct. 3, 2001, and U.S. Ser. No. 10/144,194, filed May 14, 2002.

### DESCRIPTION OF THE DRAWINGS

FIGS. 1-20 show amino acid sequence alignments between polypeptides of the present invention, and polypeptides listed in public databases. SEQ ID NOS for the polypeptides of the present invention are listed in Table 3. Others are as follows: NP_{—}065728 (SEQ ID NO 93); XM_{—}046054 (SEQ ID NO 94); XM_{—}108902 (SEQ ID NO 95); NM_{—}015046 (SEQ ID NO 96); XM_{—}045169 (SEQ ID NO 97); XM_{—}035861 (SEQ ID NO 98); NM_{—}032141 (SEQ ID NO 99); XM_{—}054973 (SEQ ID NO 100); XM_{—}047529 (SEQ ID NO 101); NM_{—}012201 (SEQ ID NO 102); NM_{—}018114 (SEQ ID NO 103); NM_{—}002026 (SEQ ID NO 104); NM_{—}016569 (SEQ ID NO 105); XM_{—}051146 (SEQ ID NO 106); AAK83466 (SEQ ID NO 107); AAF19255 (SEQ ID NO 108); AAC97961 (SEQ ID NO 109); AK000274 (SEQ ID NO 110); NM_{—}016648 (SEQ ID NO. 111); BAA03747 (SEQ ID NO 112); XM_{—}047995 (SEQ ID NO 113); XM_{—}031992 (SEQ ID NO 114).

FIG. 21 shows the expression pattern of genes of the present invention in normal breast (top panel) and breast cancer (bottom panel) tissues. Twelve different tissue samples were analyzed for each gene using RT-PCR with gene-specific primers. In each case, gene expression was up-regulated in the cancer. The arrowhead indicates the position of the PCR product which corresponds to the gene. The lower less defined bands represent the primer dimers.

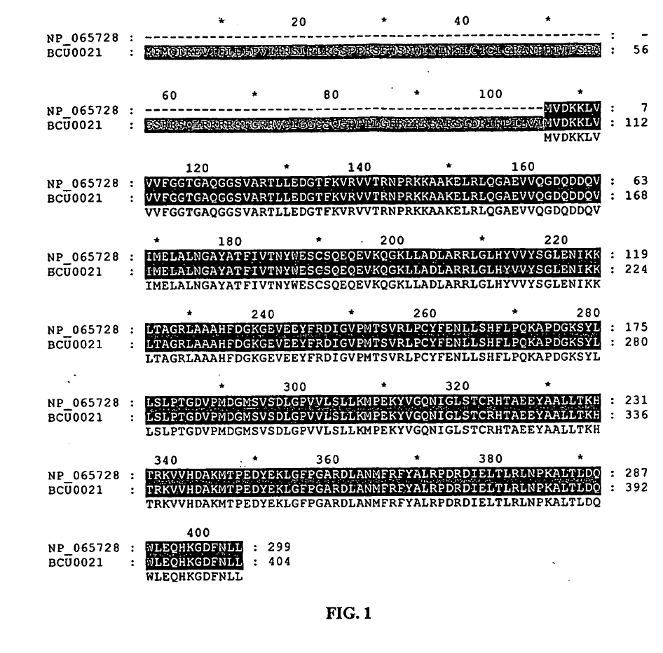

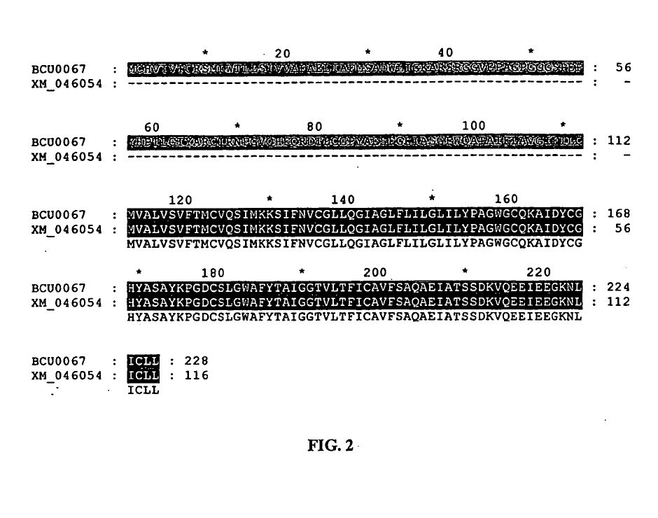

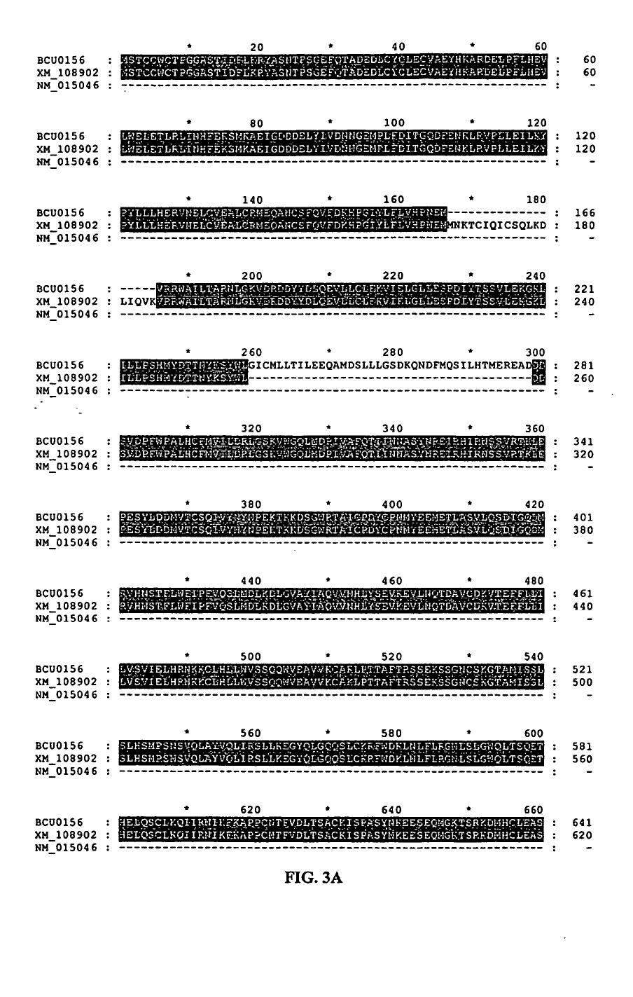

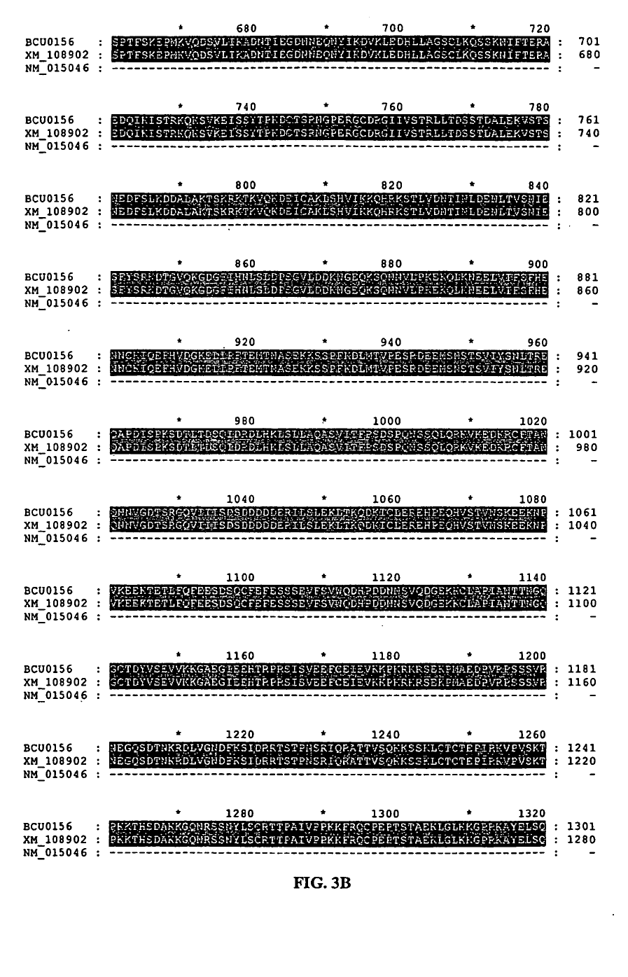

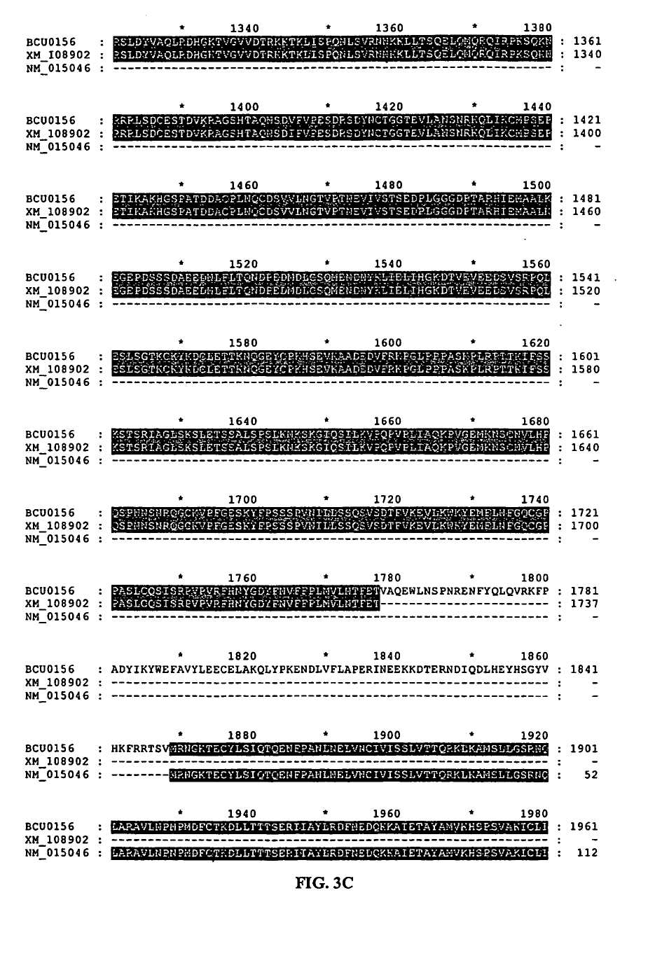

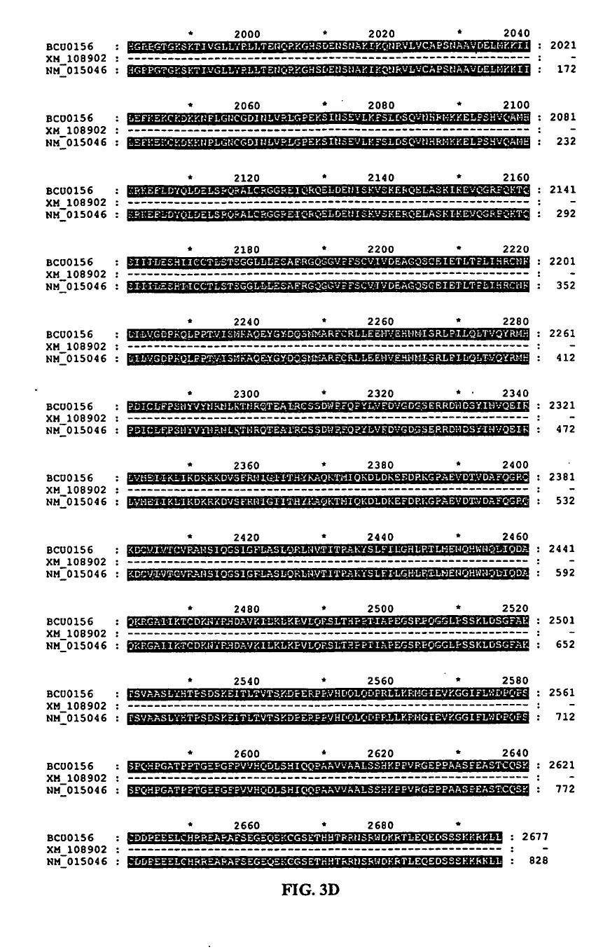

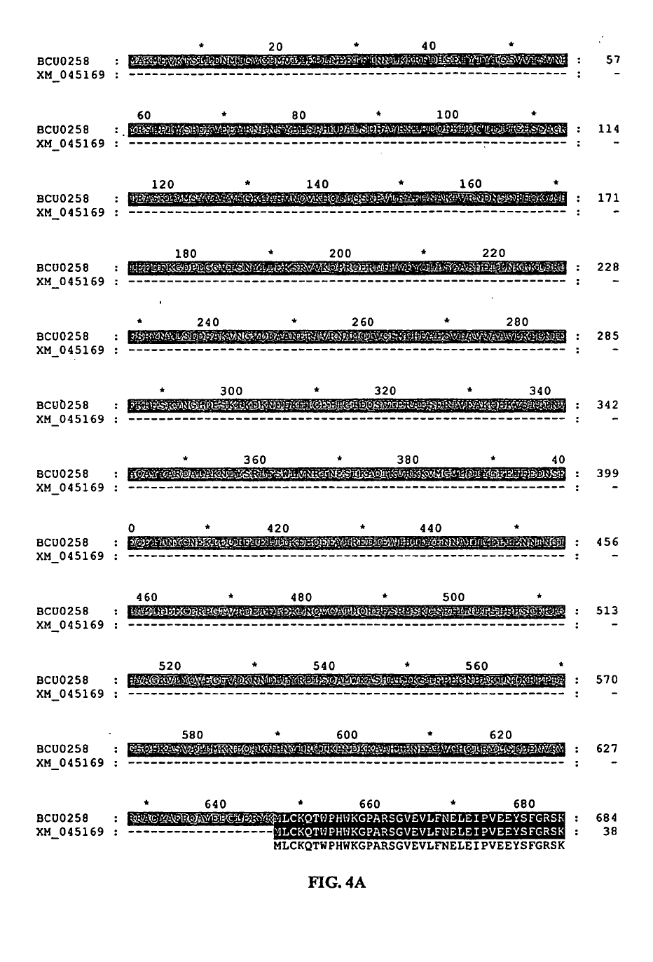

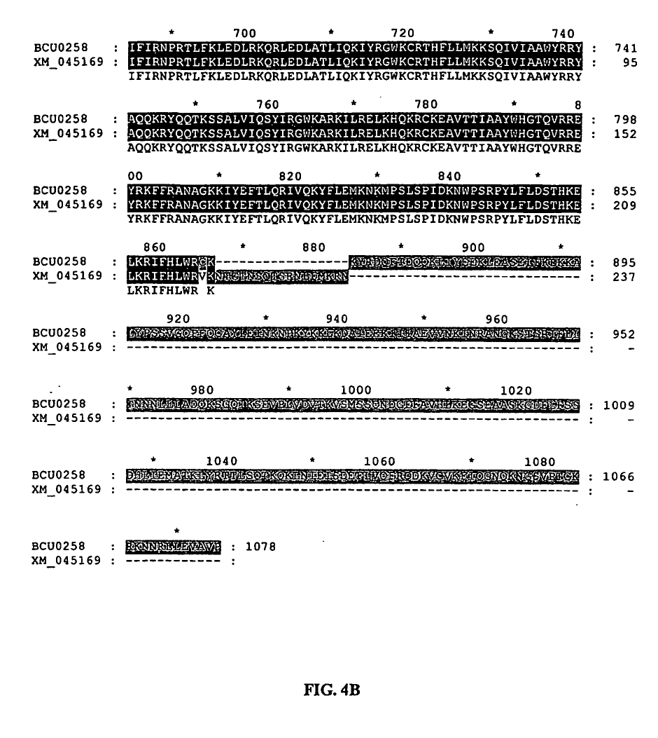

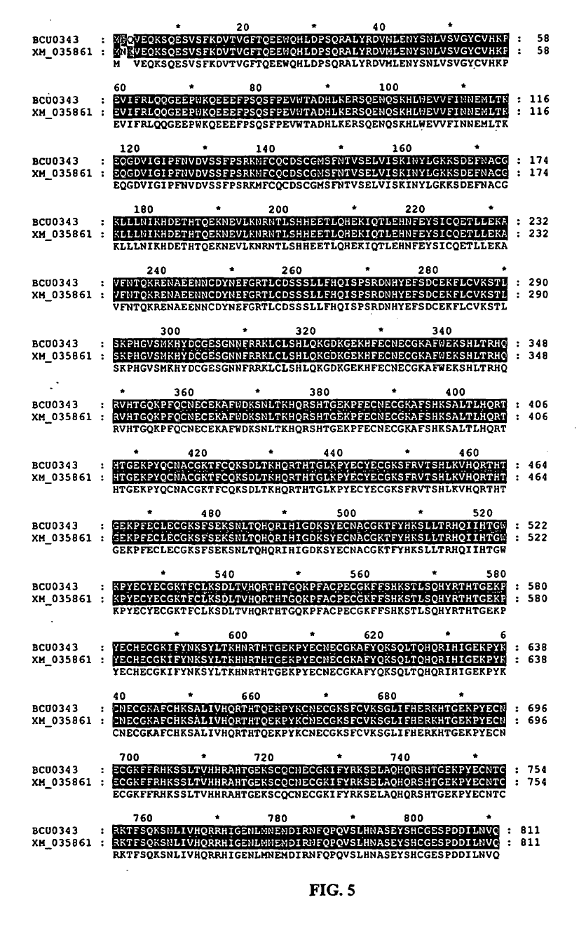

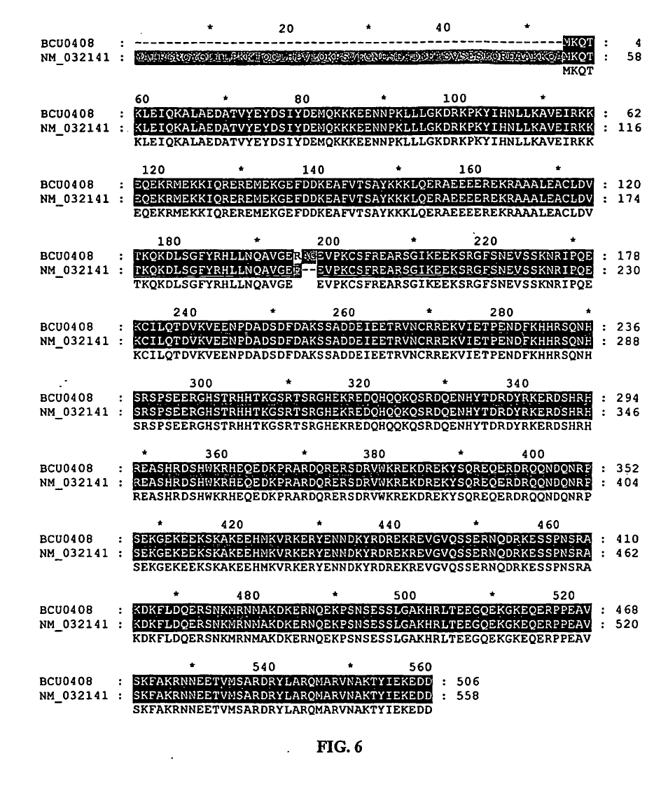

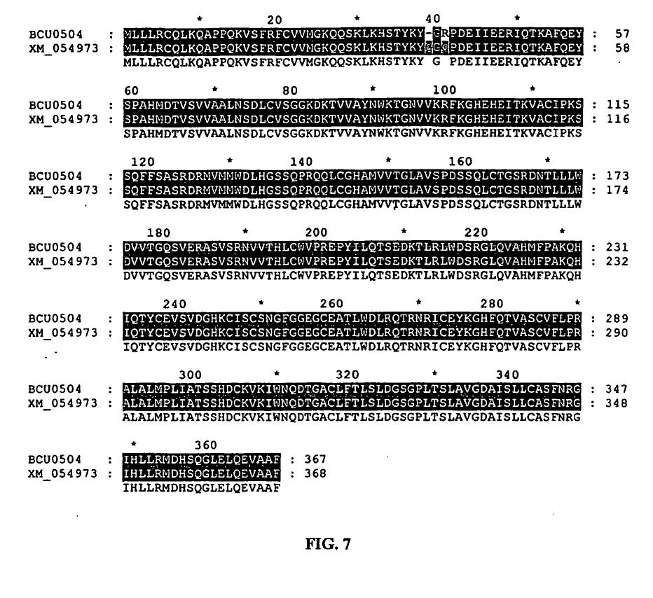

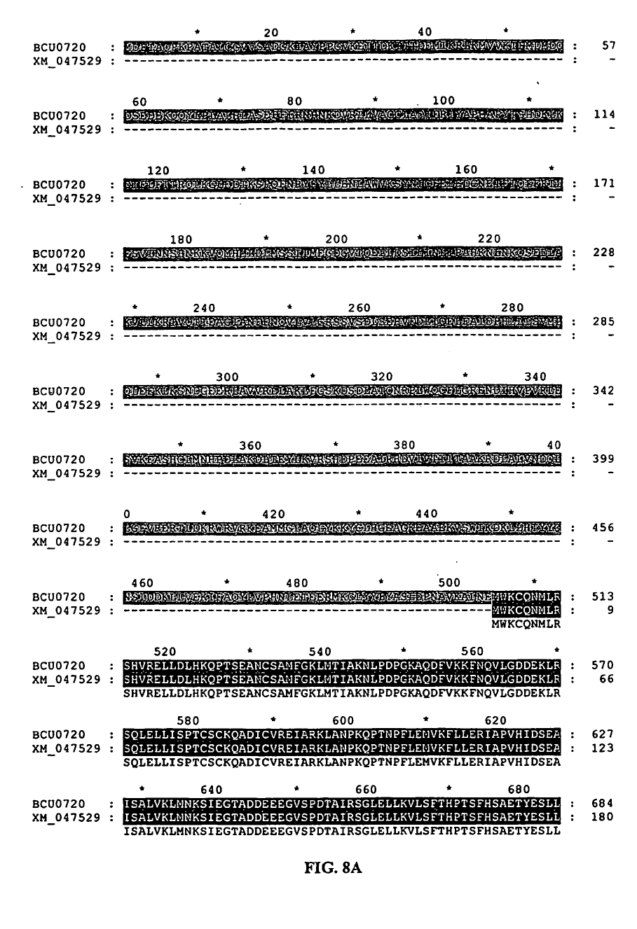

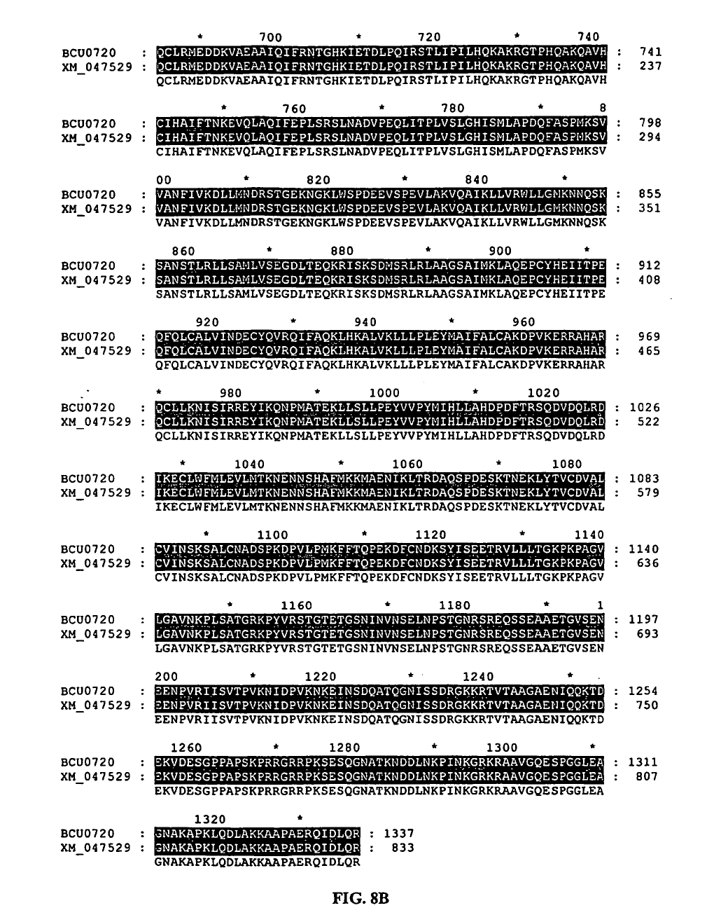

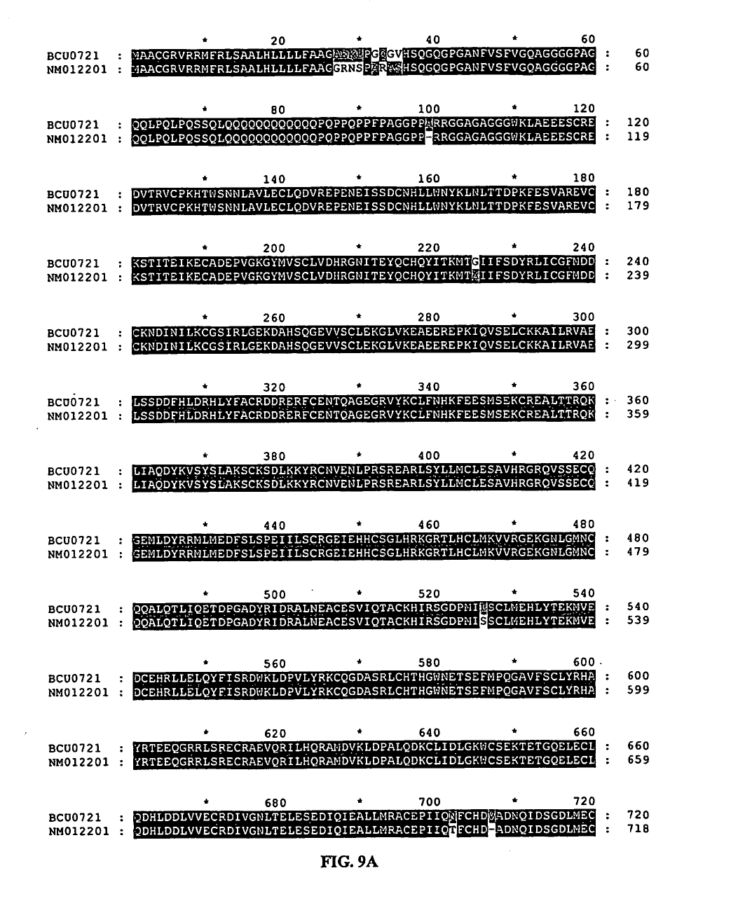

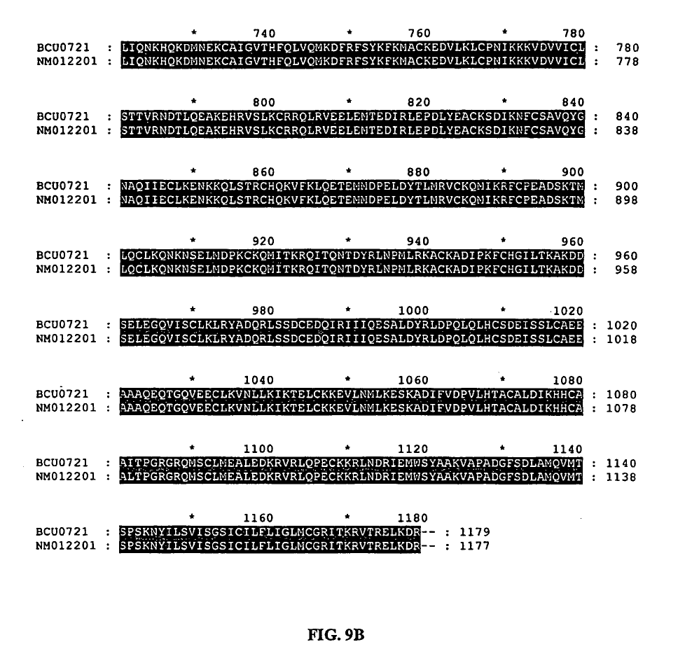

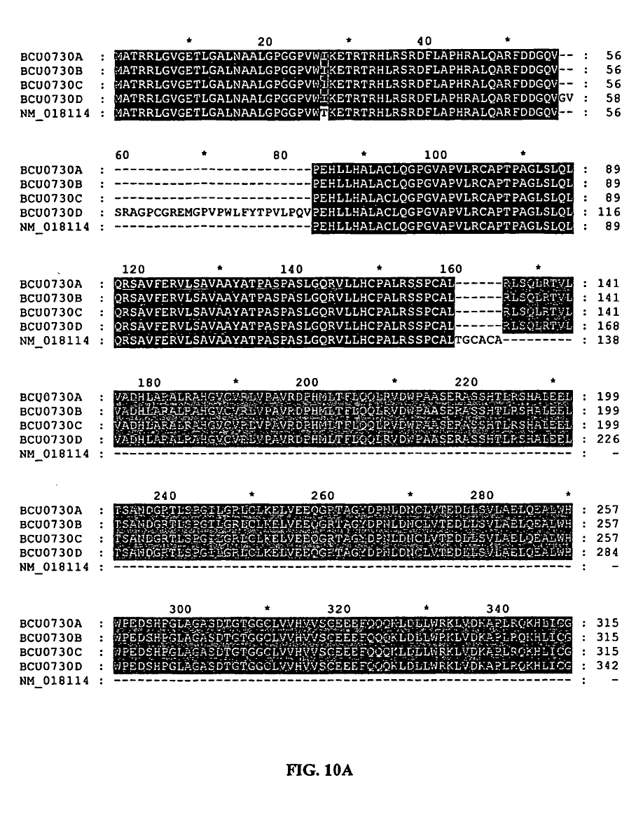

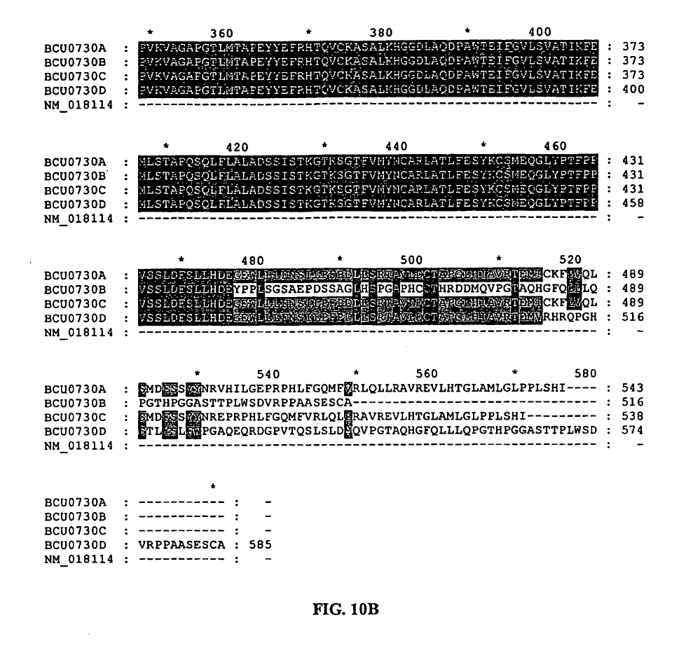

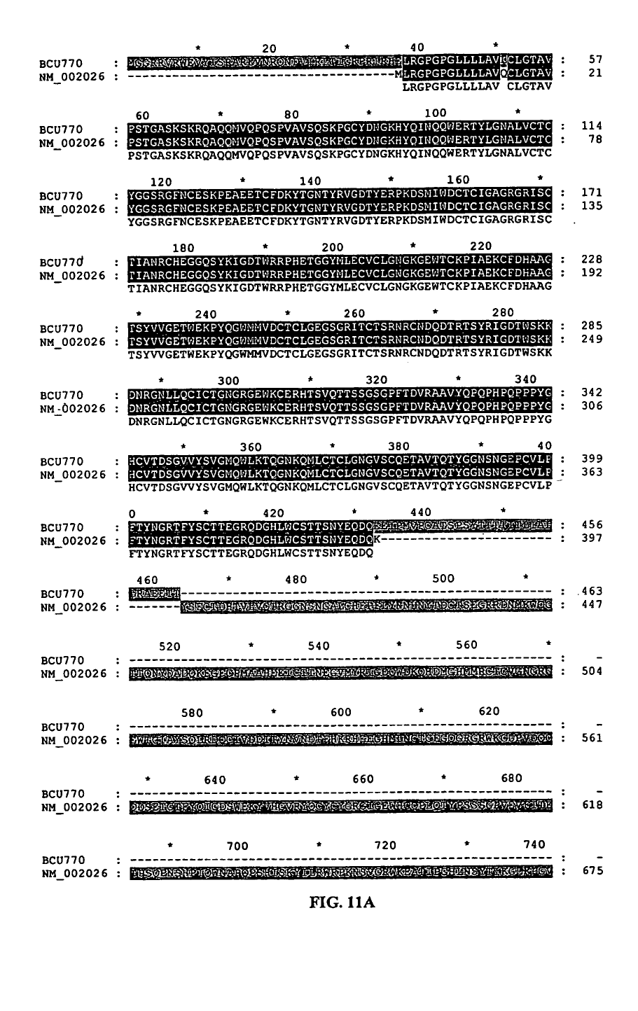

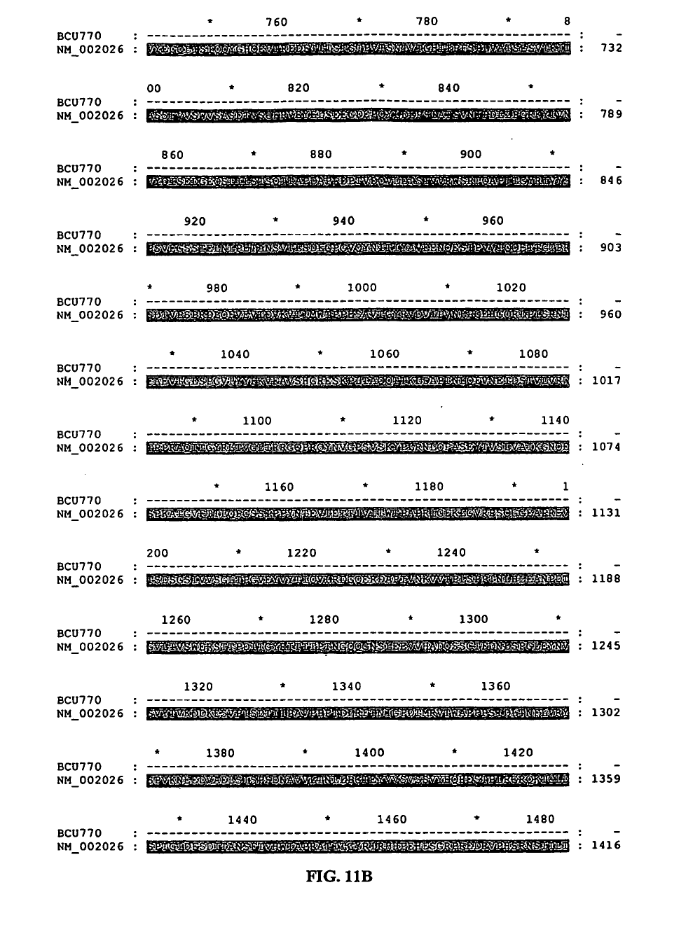

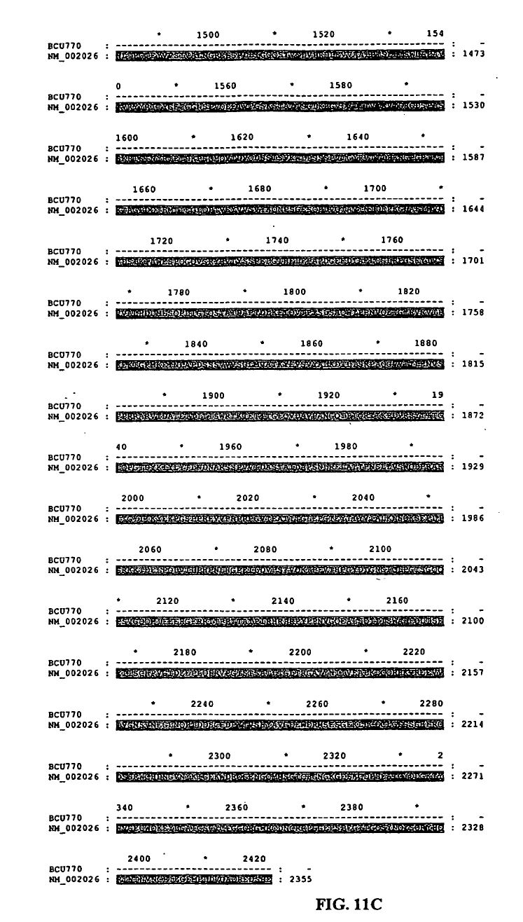

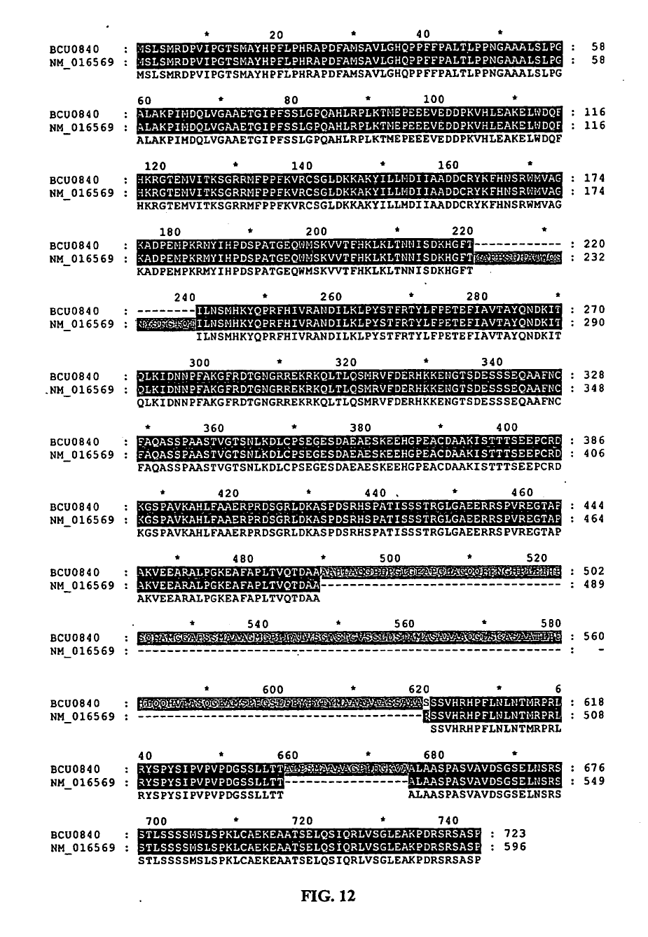

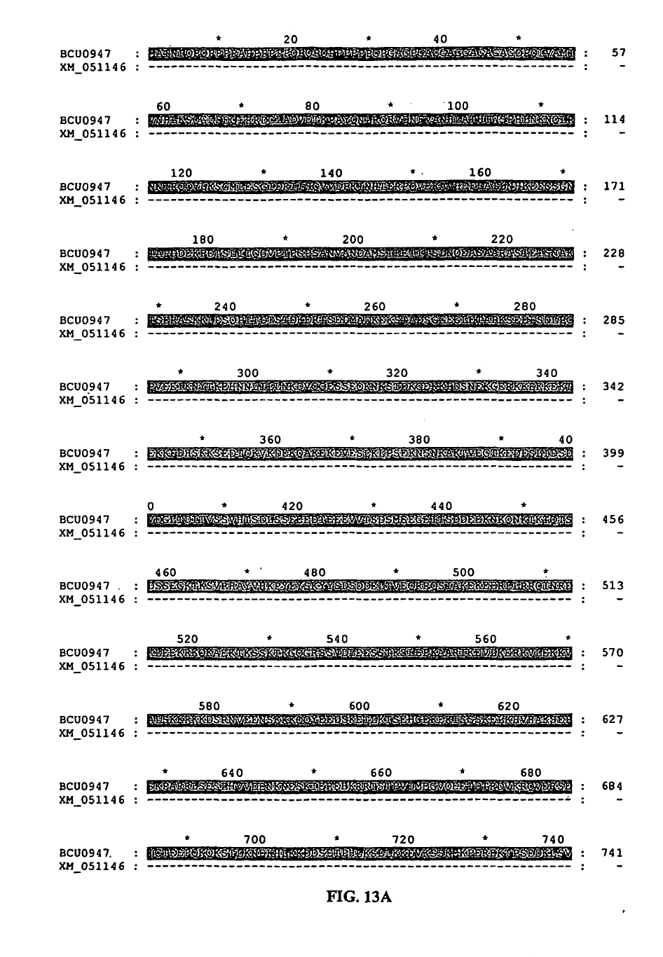

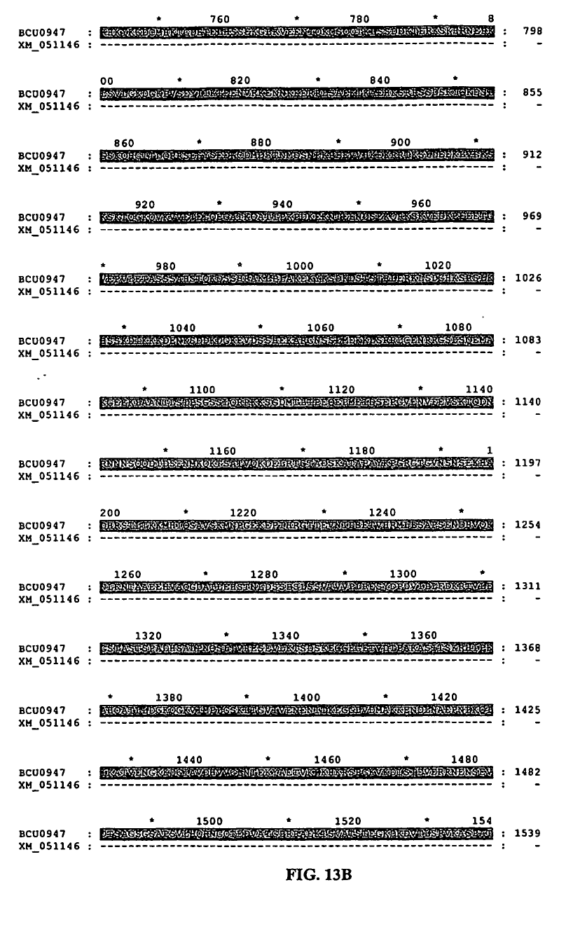

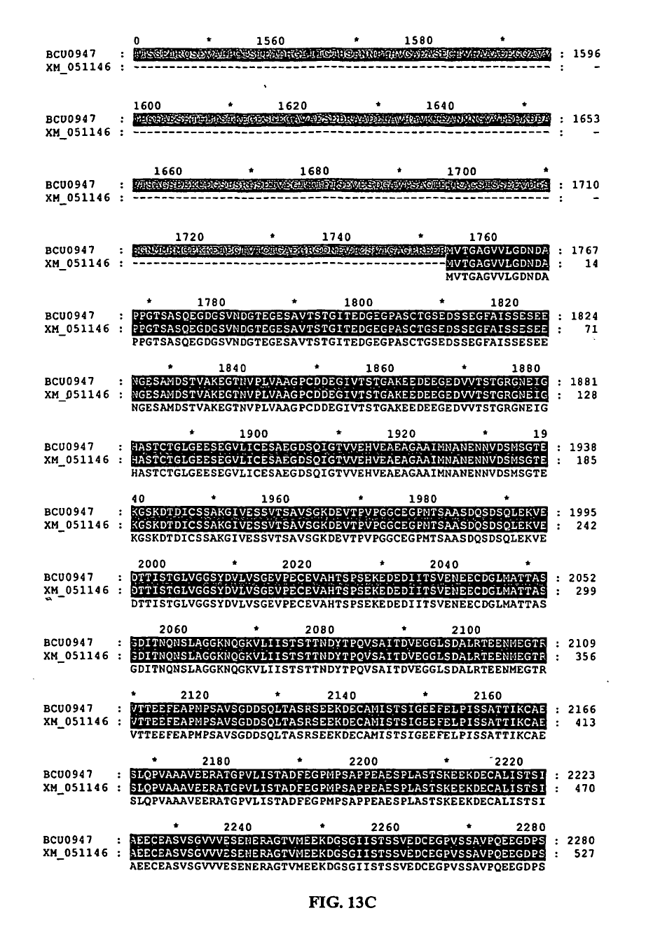

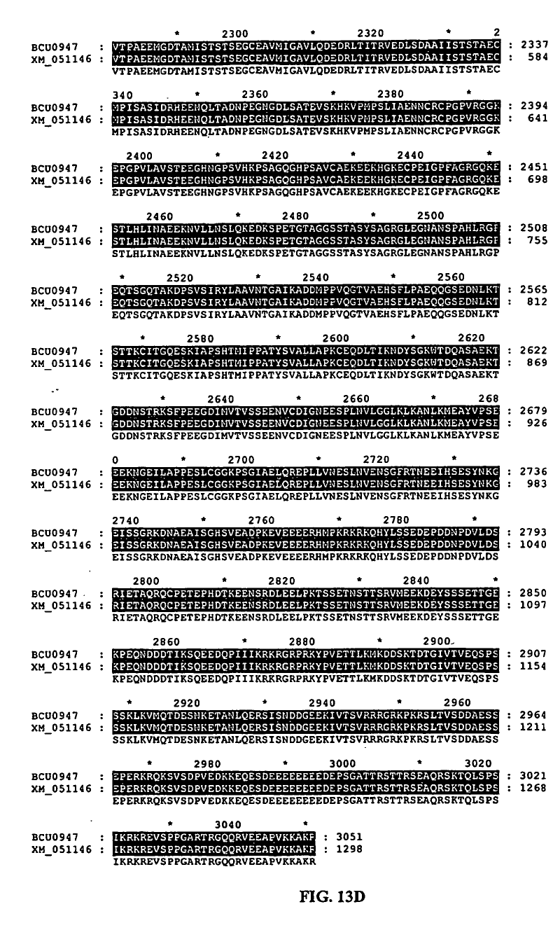

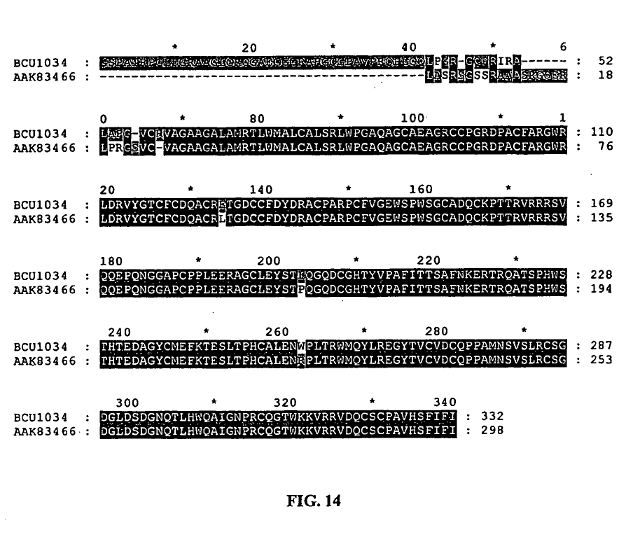

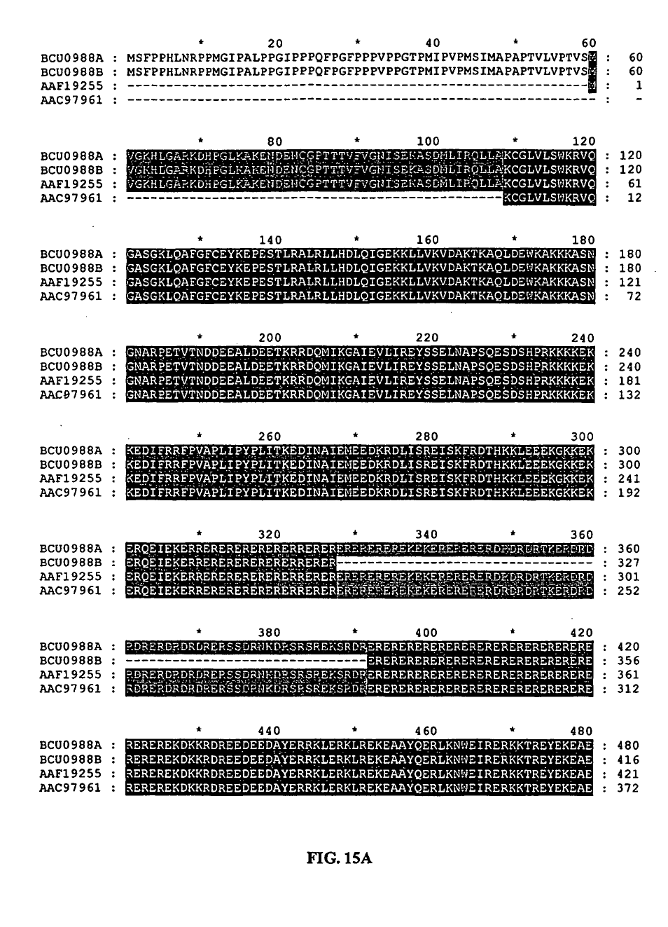

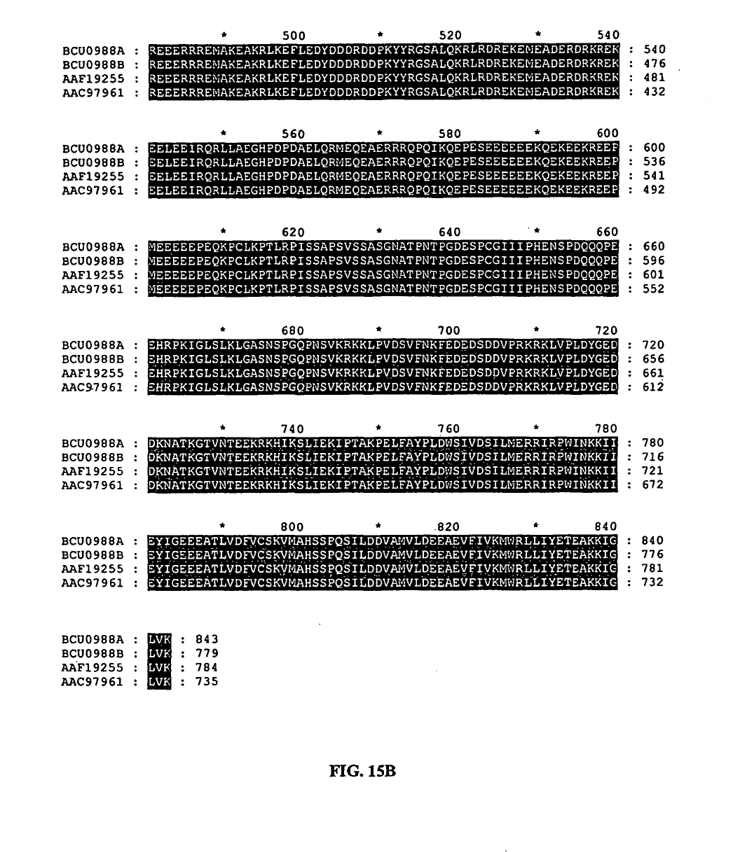

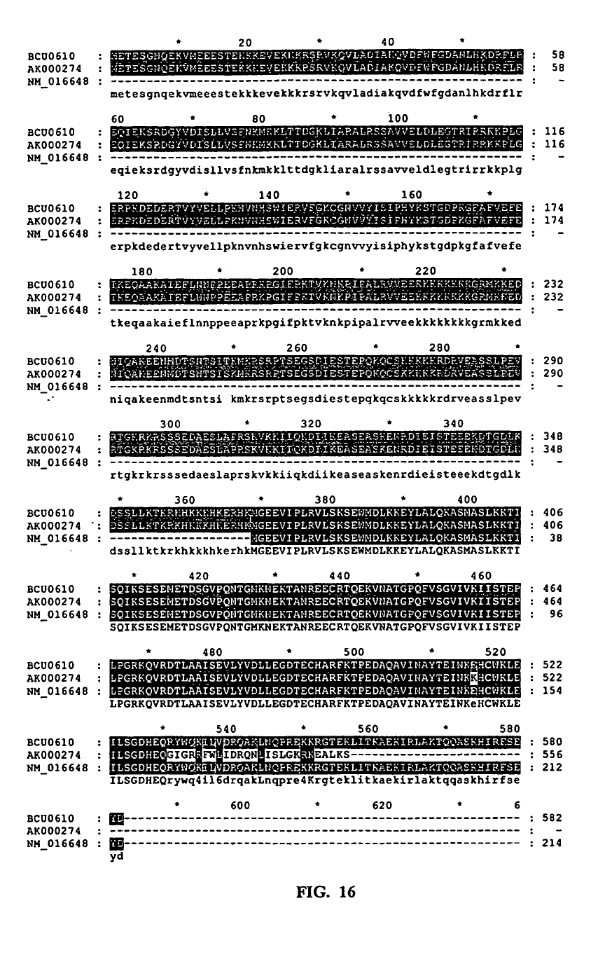

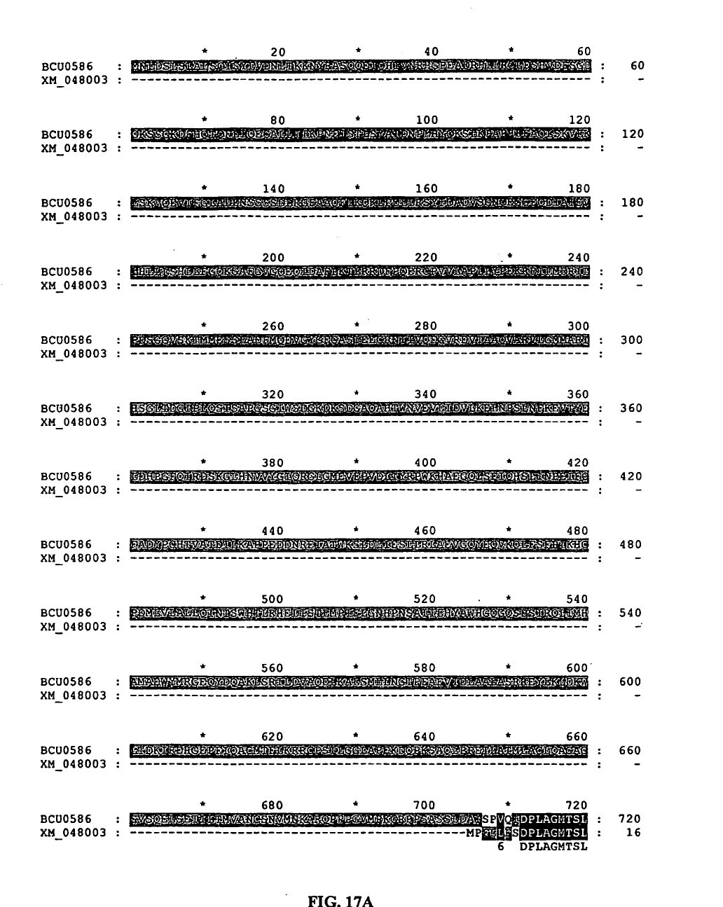

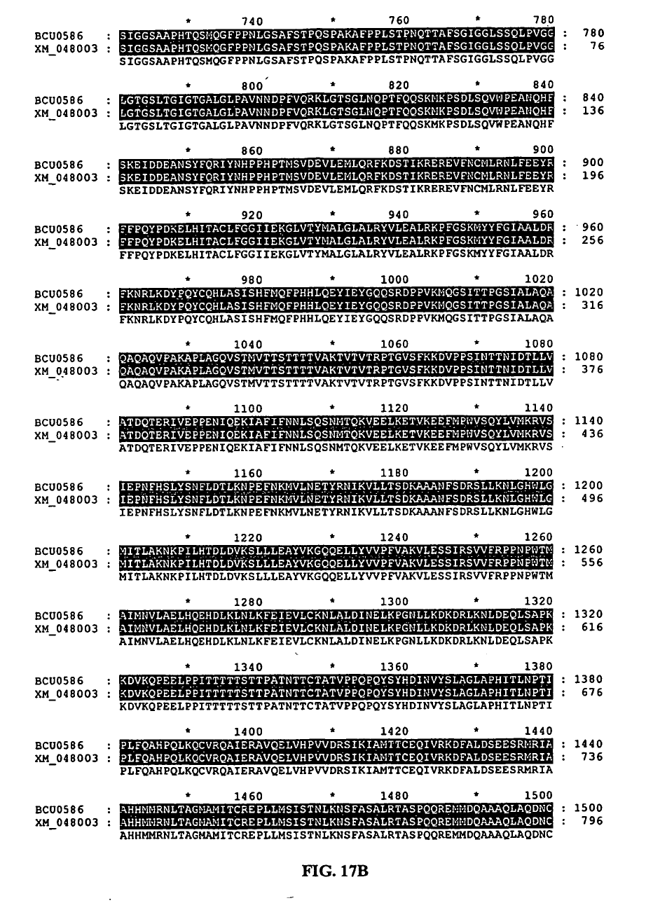

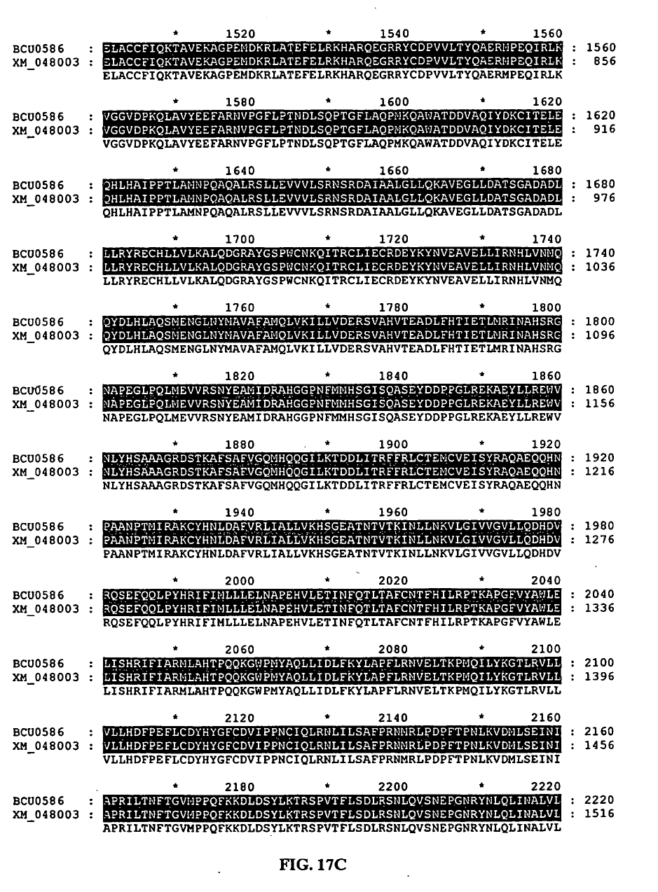

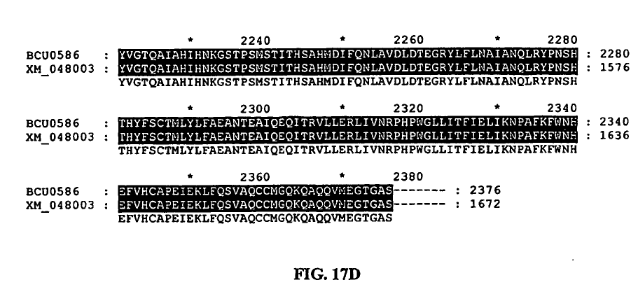

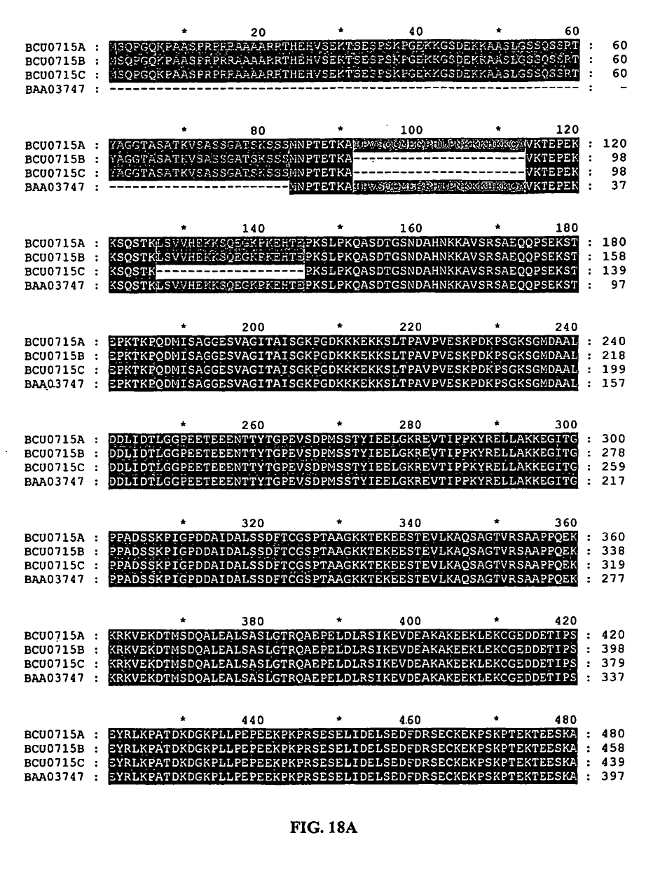

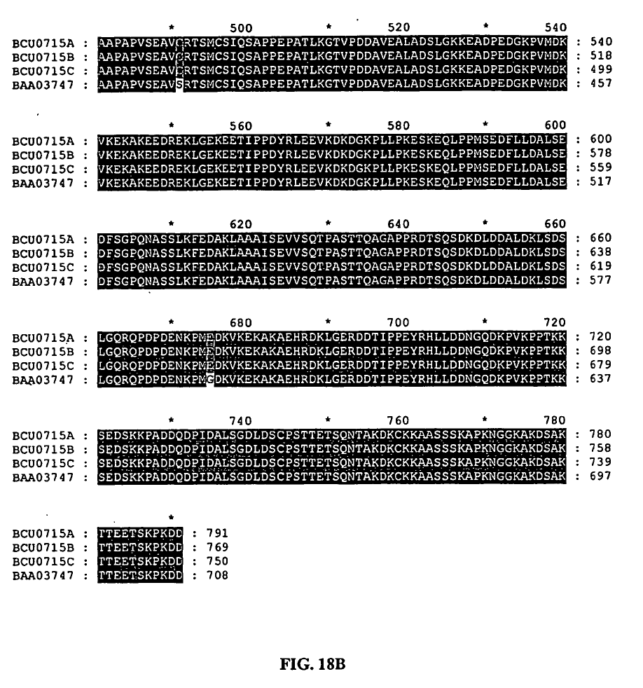

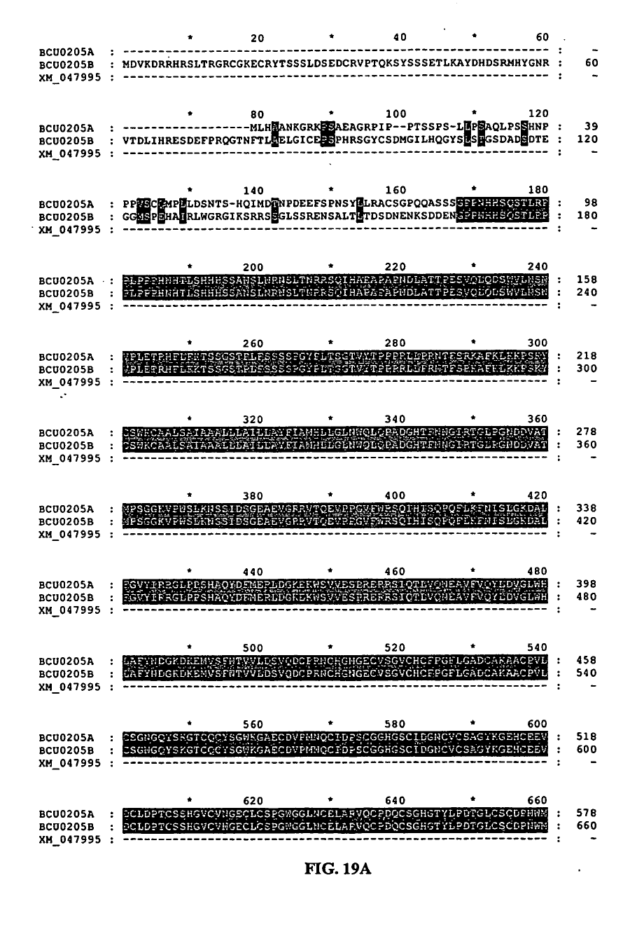

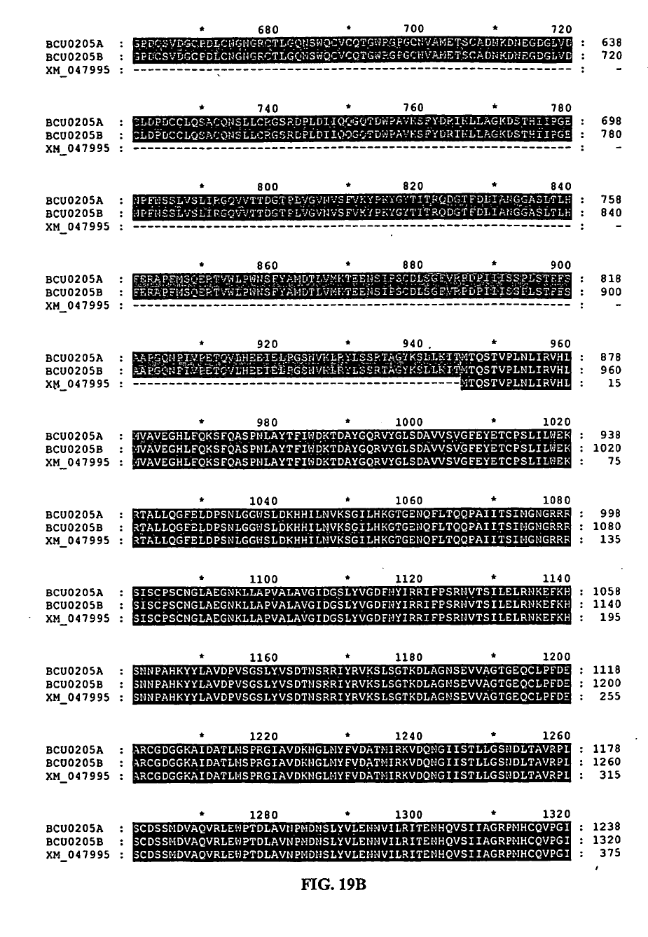

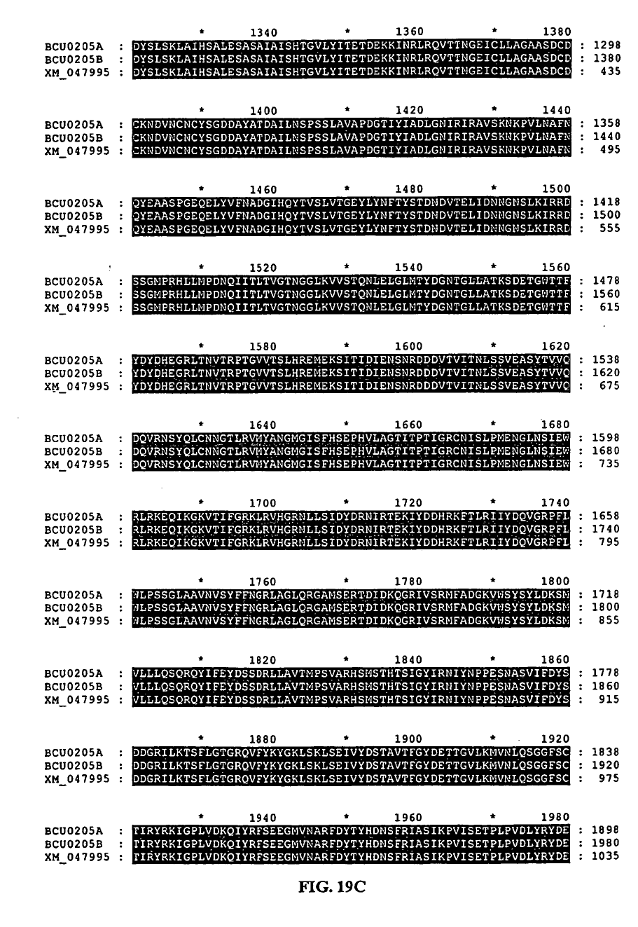

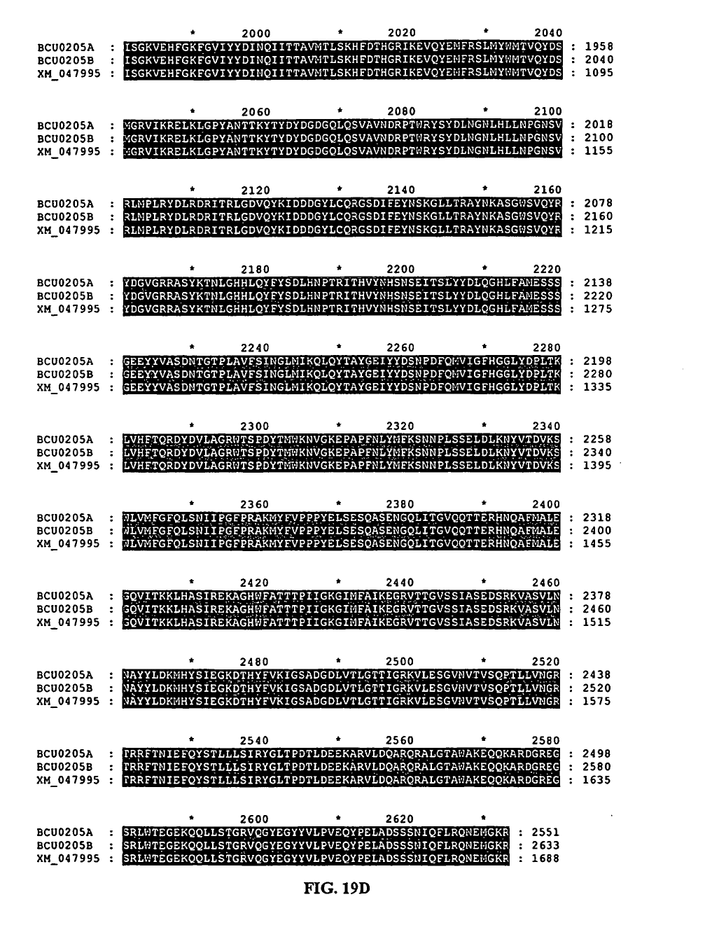

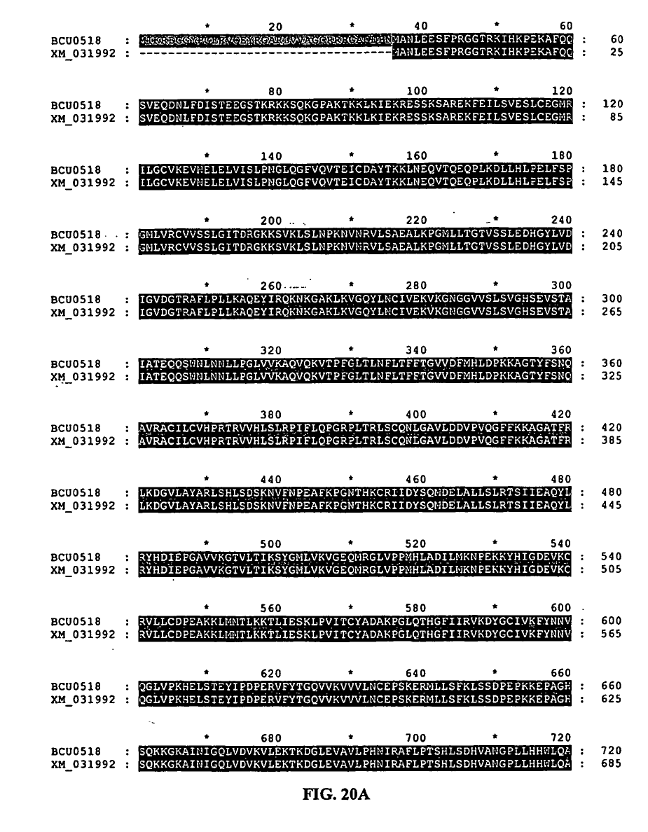

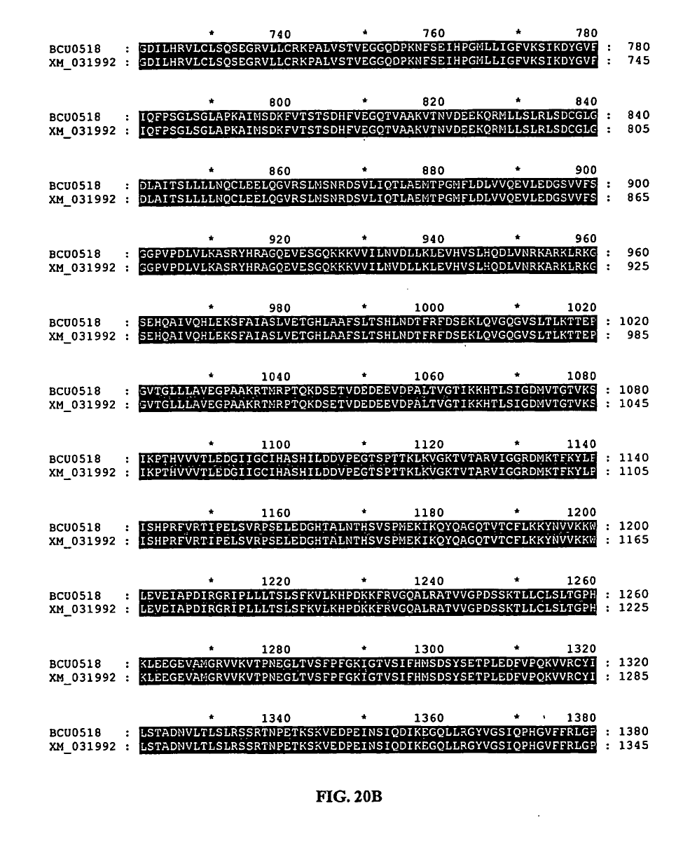

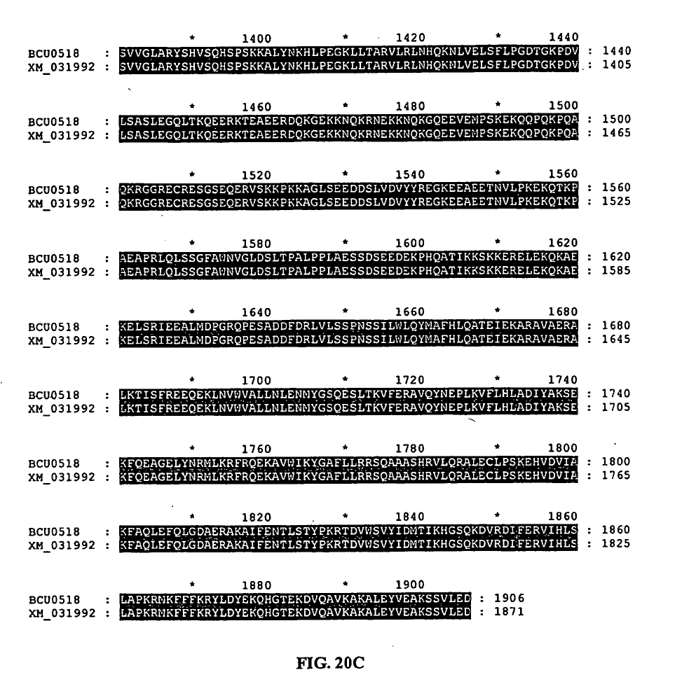

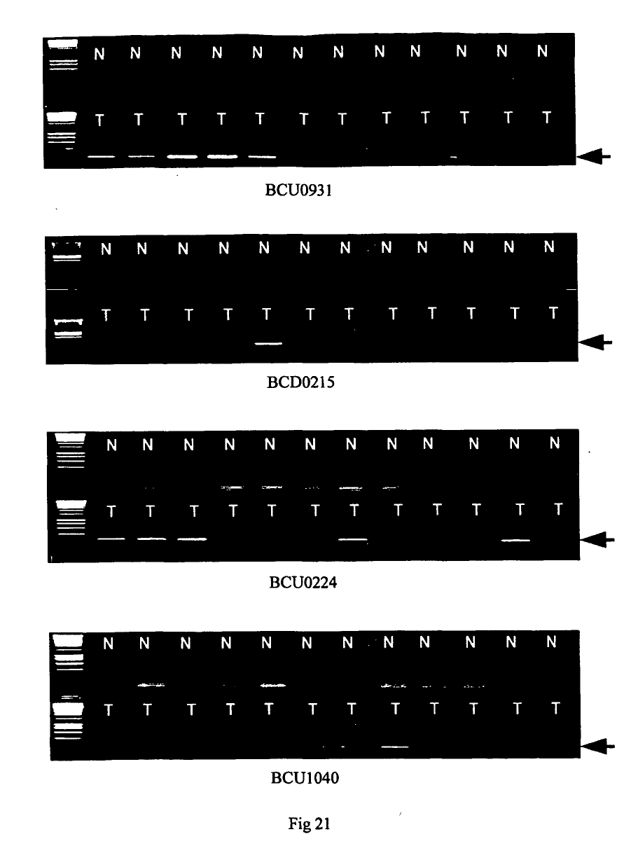

### DESCRIPTION OF THE INVENTION

The present invention relates to all facets of novel polynucleotides, the polypeptides they encode, antibodies and specific binding partners thereto, and their applications to research, diagnosis, drug discovery, therapy, clinical medicine, forensic science and medicine, etc. The polynucleotides are differentially regulated in breast cancer and are therefore useful in variety of ways, including, but not limited to, as molecular markers, as drug targets, and for detecting, diagnosing, staging, monitoring, prognosticating, preventing or treating, determining predisposition to, etc., diseases and conditions especially relating to breast cancer, but to other diseases and disorders, as well. The identification of specific genes, and groups of genes, expressed in pathways physiologically relevant to breast cancer permits the definition of functional and disease pathways, and the delineation of targets in these pathways which are useful in diagnostic, therapeutic, and clinical applications. The present invention also relates to methods of using the polynucleotides and related products (proteins, antibodies, etc.) in business and computer-related methods, e.g., advertising, displaying, offering, selling, etc., such products for sale, commercial use, licensing, etc.

Breast Cancer

Breast cancer is the second leading cause of cancer death for all women (after lung cancer), and the leading overall cause of death in women between the ages of 40 and 55. In 2000, several hundred thousand new cases of female invasive breast cancer were diagnosed, and about 40,000 women died from the disease. Nearly 43,000 cases of female in situ (preinvasive) breast cancer were diagnosed in 2000. There is not one single disease that can be called breast cancer. Instead, it is highly heterogeneous, exhibiting a wide range of different phenotypes and genotypes. No single gene or protein has been identified which is responsible for the etiology of all breast cancers. It is likely that diagnostic and prognostic markers for breast cancer disease will involve the identification and use of many different genes and gene products to reflect its multifactorial origin.

The normal female breast comprises ducts and lobuloalveolar structures surrounded by basement membranes and collagenous stroma with fibroblasts, vessels, and fat. The basic unit of function in the breast are the lobuloalveolar structures which produce the milk secretions. Each lobule drains into a lactiferous duct that empties into a lactiferous sinus beneath the nipple. The ducts are lined with epithelial cells, containing few mitochondria and sparse endoplasmic reticulum. The lobules contain luminal epithelial cells, basal epithelial cells, and myoepithelial cells. The basal and epithelial cells are sometimes grouped together. The luminal cells can be differentiated immuno-histochemically from the myoepithelial cells by their expression of keratins. The luminal cells stain with antibodies to keratin 5/6; the myoepithelial cells stains with antibodies against keratin 8/18. In addition to the presence of these cells types in the breast, there are endothelial cells associated with blood vessels, stromal cells that surround the lobular structures, adipose cells, and blood cells, such as T-lymphocytes and macrophages.

Breast carcinoma can be classified into two basic types, noninvasive (non-infiltrating) and invasive. Noninvasive carcinoma includes, e.g., intraductal carcinoma (also known as ductal carcinoma in situ or “DCIS”), intraductal papillary carcinoma, and lobular carcinoma in situ. Invasive carcinoma includes, e.g., invasive ductal carcinoma (“IDC”), invasive lobular carcinoma, medullary carcinoma, colloid carcinoma (mucinous carcinoma), Paget's disease, tubular carcinoma, adenoid cystic carcinoma, invasive comedocarcinoma, apocrine carcinoma, and invasive papillary carcinoma. See, also, Cancer, Principles and Practice of Oncology, DeVita et al., ed., J. B. Lippincott Company, 1982, Pages 914-922. The different cancers can generally be distinguished histologically from each other.

Over 90% of breast cancers arise in the ducts. As long as it remains with the ductal basement membranes, it is classified as a non-infiltrating or non-invasive carcinoma. DCIS is a common example. An invasive or infiltrating carcinoma shows a marked increase in dense fibrous tissue stroma, giving the tissue a hard consistency. IDC is one of the more common types of an invasive carcinoma. Frequently, an infiltrating carcinoma becomes invaded with blood and lymphatic vessels as it increases in size and malignancy. The tumor cells fill the ducts, plugging them, and invade the surrounding stroma. For general description of breast pathology, see, e.g., Robins Pathological Basis of Disease, Cotran et al., 4^{th} Edition, W. B. Saunders Company, 1989, Chapter 25.

The progression of a cancer, from its origin to a full-blown malignancy, is the subject of intense study. Hyperplasia is generally believed to precede at least some cancers, but not all hyperplasia leads to cancer, and the relationship between the two is not well understood. One hallmark of a hyperplasia that leads to cancer may be the occurrence of genomic instability, and other factors which lead to uncoupling of the cell cycle.

Intraepithelial neoplasia is one of the first detectable signs of a breast cancer, characterized by its confinement to the duct epithelia. It can also be referred to as preinvasive neoplasia, precancer, dysplasia, or CIS. See, e.g., Boone et al., Proc. Soc. Exp. Biol. Med., 216:151-165, 1997. An intraepithelial neoplasia generally consists of multiple foci of an abnormal clonal expansion of neoplastic cells. The development of the neoplasia is manifested by an increasing size of the lesion and a greater degree of cytonuclear morphological aberration, as it progresses from low grade to high grade. See, e.g., Bacus et al., Cancer Epid. Biom. Prevent., 8:1087-1094, 1999. An early grade can be referred to as an intraductal proliferation (IDP). More advanced, pre-invasive lesions are DCIS and LCIS (lobular carcinoma in situ). It is believed that DCIS and LCIS are precursor lesions of invasive breast cancer, such as IDC. See, e.g., Buerger al., Mol. Pathol., 53:118-121, 2000.

Breast cancers can be both staged and graded. Stage is based on the tumor and size and whether the lymph nodes are involved with the tumor. Tumor grade refers to the tumor cells' appearance under the microscope, and how closely it resembles normal tissue of the same type. If the tumor cells look normal, then it can be termed “low grade.” High grade cells look markedly different from normal cells. High grade tumors tend to behave more aggressively than lower grade.

The most widely used clinical staging system for breast cancer is one adopted by the UICC (International Union against Cancer). This system incorporates the TNM (t, tumor; N, nodes; M, metastases) classification using tumor size, involvement of the chest wall and skin, inflammatory cancer, involvement of nodes, evidence of metastases. See, e.g., Sainsbury et al., BMJ, 321:745-750, 2000. Other staging and grading systems can also be used, e.g., Bloom and Richardson grade (British J. Cancer, 11:359-377, 1957), Columbia Clinical Classification (CCC), Van Nuys (VN), etc. Grading systems have also been devised based on image analysis of neoplastic and normal cells. Bacus et al. (Cancer Epid. Biom. Prevent., 8:1087-1094, 1999) have described an image morphometric nuclear grading system for intraepitheliam neoplastic lesions, such as DCIS, which provides objective criteria to assess tumor grade. See, also, Schwartz, Human Pathol., 28:1798-1802, 1997, for a grading system for DCIS. FISH has also been used to diagnose cancers based on chromosomal aberrations. See, e.g., Komoike et al., Breast Cancer, 7:332-336, 2000.

Various genetic bases for breast cancer have begun to be identified. For instance, BRCA1, BRCA2, ATM, PTEN/MMAC1 (e.g., Ali et al., J. Natl. Cancer Inst., 91:1922-1932, 1999), MLH2, MSH2, TP53 (e.g., Done et al., Cancer Res., 58:785-789, 1998), and STK11 are associated with a higher risk of cancer. Other genes involved in breast cancer include, e.g., myc, cyclin D1(e.g., Weinstat-Saslow et al., Nature Med., 1:1257-1260, 1995), and c-erb-B2.

A continuing goal is to characterize the gene expression patterns of the various carcinoma forms in order to genetically differentiate them, providing important guidance in preventing and treating cancer. For instance, the c-erb-B2 gene codes for a transmembrane protein which is over-expressed in about 20-30% of all breast cancers. Based on this information, immunotherapy using an anti-c-erb-B2 antibody has been developed and successfully used to treat breast cancer. See, e.g., Pegram and Slamon, Semin Oncol., 5, Suppl 9:13, 2000. Molecular pictures of cancer, such as the pattern of up-regulated genes identified herein, provide an important tool for molecularly dissecting and classifying cancer, identifying drug targets, providing prognosis and therapeutic information, etc. For instance, an array of polynucleotides corresponding to genes differentially regulated in breast cancer can be used to screen tissue samples for the existence of cancer, to categorize the cancer (e.g., by the particular pattern observed), to grade the cancer (e.g., by the number of up-regulated genes and their amounts of expression), to identify the source of a secondary tumor, to screen for metastatic cells, etc. These arrays can be used in combination with other markers, e.g., keratin immunophenotyping (e.g., CK 5/6), c-erb-B2, estrogen receptor (ER) status, etc., and any of the grading systems mentioned above.

Tables 1 and 4 list differentially regulated genes, the cellular locations of the polypeptides coded for by the genes, and their corresponding functional and structural polypeptide domains. Table 3 summarizes the expression profile of these genes.

Membrane (i.e., cell-surface) proteins coded for by up-regulated genes (e.g., BCU0067, BCU0149, BCU0721, BCU41, BCU770, BCU224; see, Table 1 and 4 for others) are useful targets for antibodies and other binding partners (e.g., ligands, aptamers, small peptides, etc.) to selectively target agents to a breast cancer tissue for any purpose, included, but not limited to, imaging, therapeutic, diagnostic, drug delivery, gene therapy, etc. For example, binding partners, such as antibodies, can be used to treat carcinomas in analogy to how c-erbB-2 antibodies are used to breast cancer. Membrane (e.g., when shed into the blood and other fluid) and extracellular proteins can also be used as diagnostic markers for cancer, and to assess the progress of the disease, e.g., in analogy to how PSA levels are used to diagnose prostate cancer. Useful antibodies or other binding partners include those that are specific for parts of the polypeptide which are exposed extracellularly.

Polynucleotides of the present invention can also be used to detect metastatic cells in the blood. For instance, BCU0120, BCU0156, BCU0258, BCU0475, BCU0504, BCU0571, BCU0770, BCU0840, BCU0862, BCU0918, and BCU0205 are absent from peripheral blood cells, and can therefore be used in diagnostic tests to assess whether breast cancer cells have metastasized from the primary site.

Polynucleotides of the present invention have been mapped to specific chromosomal bands. Different human disorders are associated with these chromosome locations. See, Table 2. The polynucleotides and polypeptides they encode can be used as linkage markers, diagnostic targets, therapeutic targets, for any of the mentioned disorders, as well as any disorders or genes mapping in proximity to them. Of particular interest are those genes which map to cancer loci, such as BCU0371, BCU0720, BCU0721, BCU0730, BCU0862, and BCU0715.

BCU0715 represents alternative splice forms of a calpain inhibitor. Calpains are neutral cysteine proteases. Calpain inhibitors have a number of uses, e.g., to inhibit the formation of cataracts (e.g., Current Eye Res., 22(4): 280-285, 2001), to treat brain ischemia (e.g., NeuroReport, 12:3927-3931, 1999), to treat and/or prevent neuronal damage, including brain, ear, eye, and other sensory organs (e.g., Brain Res., 850(1-2): 23443, 1999), to treat and/or prevent muscle degeneration (e.g., Stracher, Ann. N.Y. Acad. Sci., 884:52-9, 1999), to treat and/or prevent tinnitus (Shulman, Int. Tinnitus J., 4(2): 134-140, 1998), to treat myocardial injury, to identify calpain inhibitors, etc. Calpain inhibitory activity can be measured routinely, e.g., by measuring the inhibition of a neutral cysteine proteases (see, e.g., Wronski et al., J. Neural Transm., 107:145-157, 2000; Meyer et al., Biochem. J., 314 (Pt. 2):511-519, 1996).

Bcd215

Bcd215 (related to NM_{—}001871) codes for a carboxypeptidase having 251 amino acids. The nucleotide and amino acid sequences of Bcd215 are shown in SEQ ID NOS 125-126. It contains a Zn_pept domain at about amino acid positions 119-229, and a carboxypeptidase activation domain at about amino acid positions 26-105.

All or part of Bcd215 is located in genomic DNA represented by AC024897, BAC-ID: RP11-505J9, and NT_{—}005616. The gene contains at least 8 exons. The present invention relates to any isolated introns and exons that are present in such clone. Such introns and exons can be routinely determined. Bcd215 maps to chromosomal band 3q24.1. As indicated by its expression profile, Bcd215 is down-regulated in breast cancer. In addition to its role in breast cancer, Bcd215 is also expressed in other tissues, and thus is associated with other phenotypes, including other diseases and conditions. These, include, e.g., Hermansky-Pudlak syndrome, leukemia and other types of cancers, including adenocarcinoma, Usher syndrome, and Seckel syndrome. Nucleic acids of the present invention can be used as linkage markers, diagnostic targets, therapeutic targets, for any of the mentioned disorders, as well as any disorders or genes mapping in proximity to it.

Bcu41

Bcu41 codes for a 98 amino acid transmembrane polypeptide. The nucleotide and amino acid sequences of Bcu41 are shown in SEQ ID NOS 115-116. The transmembrane domain is located amino acid positions 65-87, and there is a Zinc carboxypeptidaseA metalloprotease (M14 family) motif at amino acids 3646. It is also related to mouse AK018709.

All or part of Bcu41 is located in genomic DNA represented by GenBank ID: AC040977, BAC ID: RP11-589P10, and Contig ID: NT_{—}010747. The gene contains at least three exons. The present invention relates to any isolated introns and exons that are present in such clone. Such introns and exons can be routinely determined. Bcu41 maps to chromosomal band 17q13.1, coincident with the location of a breast cancer tumor suppressor gene.

Bcu259

Bcu259 (related to NM_{—}032926) codes for a nuclear protein containing 200 amino acids. The nucleotide and amino acid sequences of Bcu259 are shown in SEQ ID NOS 131-132. It contains a bipartite nuclear localization signal domain at about amino acids 96-116.

All or part of Bcu259 is located in genomic DNA represented by GenBank ID: AC025230, BAC-ID: RP11-193B20, GenBank ID: Z73965 (DXS366-DXS87), and BAC-ID: Cosmid V857G6. The gene contains at least 3 exons. The present invention relates to any isolated introns and exons that are present in such clone. Such introns and exons can be routinely determined. Bcu259 maps to chromosomal band Xq22-q23.

As indicated by its expression profile, Bcu259 is differentially-regulated in breast cancer. In addition to its role in breast cancer, Bcu259 is also expressed in other tissues, and thus is associated with other phenotypes, including other diseases and conditions. These, include, e.g., Mohr-Tranebjaerg syndrome, perceptive congenital deafness X-linked 2, and fatal X-linked ataxia with deafness and loss of vision. Nucleic acids of the present invention can be used as linkage markers, diagnostic targets, therapeutic targets, for any of the mentioned disorders, as well as any disorders or genes mapping in proximity to it.

Bcu656

Bcu656 is a noncoding transcript. The nucleotide sequence of Bcu0656 is shown in SEQ ID NOS 117. All or part of Bcu0656 is located in genomic DNA represented by GenBank ID: AC007444,BAC ID: RP11-340F1, and Contig ID: NT_{—}007819. Bcu656 maps to chromosomal band 7p14.

Bcu924

Bcu924 codes for a polypeptide containing 88 amino acids. The nucleotide and amino acid sequences of Bcu924 are shown in SEQ ID NOS 139-140.

All or part of Bcu924 is located in genomic DNA represented by GenBank ID: AC012213, BAC-ID: RP11-1C8, and Contig ID: NT_{—}008005. The present invention relates to any isolated introns and exons that are present in such gene. Such introns and exons can be routinely determined. Bcu924 maps to chromosomal band 8q22-q22.3.

Bcu93

Bcu93 codes for a ribosomal protein, L23a (RPL23A) containing 156 amino acids. The nucleotide and amino acid sequences of Bcu93 are shown in SEQ ID NOS 120-121. See, e.g., Fan et al., Immunogenetics, 44:97-103, 1996; Fan et al., Genomics, 46:234-23, 1996. Polymorphisms of it are listed in Table 2.

Bcu931

Bcu931 (related to Hs.28661 and AK000933) codes for a small Ras-related GTP-binding protein involved, e.g., in the regulation of vesicular transport with exocytic and endocytic pathways. The nucleotide and amino acid sequences (e.g., containing 366 amino acids) of Bcu931 are shown in SEQ ID NOS 123-124. It has the following domains: Rab GDP-dissociation inhibitor (GDI) domain-I at about amino acid positions 3-19, Rab GDP-dissociation inhibitor (GDI) domain-2 at about amino acid positions 3-91, Rab GDP-dissociation inhibitor (GDI) domain-3 at about amino acid positions 91-113, Rab escort (choroideraemia) protein domain-1 at about at amino acid positions 6-25, Rab escort (choroideraemia) protein domain-2 at about amino acid positions 63-80, Rab escort (choroideraemia) protein domain-3 at about amino acid positions 123-151, Rab escort (choroideraemia) protein domain-4 at about amino acid positions 255-275, Rab escort (choroideraemia) protein domain-5 at about amino acid positions 277-298, and Rab escort (choroideraemia) protein domain-6 at about amino acid positions 312-332.

All or part of Bcu931 is located in genomic DNA represented by GenBank ID: AL133390, BAC ID: RP1-317G22, and Contig ID: NT_{—}004771. The present invention relates to any isolated introns and exons that are present in the gene. Such introns and exons can be routinely determined. Bcu931 maps to chromosomal band 1q43.

Bcu1040

Bcu1040 (related to Hs.20954) codes for a 95 amino acid polypeptide. The nucleotide and amino acid sequences of Bcu1040 are shown in SEQ ID NOS 137-138. All or part of Bcu1040 is located in genomic DNA represented by GenBank ID: AC022150 and BAC ID: CTD-3099C6. Bcu1040 maps to chromosomal band 19q13.41.

As indicated by its expression profile, Bcu 1040 is differentially-regulated in breast cancer. In addition to its role in cancer, Bcu1040 is also expressed in other tissues, and thus is associated with other phenotypes, including other diseases and conditions. These, include, e.g., retinosa pigmentosa and spinocerebellar ataxia. Nucleic acids of the present invention can be used as linkage markers, diagnostic targets, therapeutic targets, for any of the mentioned disorders, as well as any disorders or genes mapping in proximity to it.

Bcu224

Bcu224 (related to NM_{—}001203) codes for bone morphogenetic protein receptor type-IB containing 502 amino acids. The nucleotide and amino acid sequences of Bcu224 are shown in SEQ ID NOS 129-130. It contains a signal peptide domain at about amino acids 1-23, an activin receptor domain at about amino acids 17-110, a transmembrane domain at about amino acids 126-148, a GS motif at about amino acids 174-204, and a serine-threonine kinase domain at about amino acids 204-491. It is homologous to mouse NM_{—}007560.

All or part of Bcu224 is located in genomic DNA represented by GenBank ID: AC023177, BAC-ID: RP11-231B13, and Contig ID: NT_{—}006279. The gene contains at least 14 exons. The present invention relates to any isolated introns and exons that are present in such clone. Such introns and exons can be routinely determined. Bcu224 maps to chromosomal band 4q22-q24.

As indicated by its expression profile, Bcu224 is differentially-regulated in breast cancer. In addition to its role in breast cancer, Bcu224 is also expressed in other tissues, and thus is associated with other phenotypes, including other diseases and conditions. These, include, e.g., Wolfram syndrome-2 and mucolipidosis II. Nucleic acids of the present invention can be used as linkage markers, diagnostic targets, therapeutic targets, for any of the mentioned disorders, as well as any disorders or genes mapping in proximity to it.

Bcu616

Bcu616 codes for a polypeptide containing 90 amino acids. The nucleotide and amino acid sequences of Bcu616 are shown in SEQ ID NOS 133-134. Polymorphisms of it are listed in Table 2. The polypeptide coded for by Bcu616 exhibits sequence identity to XM_{—}003882, AK002985, and Hs.38114.

All or part of Bcu616 is located in genomic DNA represented by AC008795, BAC-ID CTD-2052F 19, and NT_{—}006679. The gene contains at least 2 exons. The present invention relates to any isolated introns and exons that are present in such clone. Such introns and exons can be routinely determined. Bcu616 maps to chromosomal band 5p13-p14. As indicated by its expression profile, Bcu616 is differentially-regulated in breast cancer. In addition to its role in breast cancer, Bcu616 is also expressed in other tissues, and thus is associated with other phenotypes, including other diseases and conditions. These, include, e.g., severe combined immunodeficiency (T-cell negative, B-cell/natural killer cell-positive type), and other types of cancer, including lung cancers. Nucleic acids of the present invention can be used as linkage markers, diagnostic targets, therapeutic targets, for any of the mentioned disorders, as well as any disorders or genes mapping in proximity to it.

Bcu631

Bcu631 (related to Hs. 101007 and Hs.5909) codes for a polypeptide containing 85 amino acids. The nucleotide and amino acid sequences of Bcu631 are shown in SEQ ID NOS 135-136.

All or part of Bcu631 is located in genomic DNA represented by AC002394, BAC-ID CIT987SK-A-211C6, and NT_{—}027181. The gene contains at least 2 exons. The present invention relates to any isolated introns and exons that are present in such clone. Such introns and exons can be routinely determined. Bcu631 maps to chromosomal band 16p12.3-12.1.

As indicated by its expression profile, Bcu631 is differentially-regulated in breast cancer. In addition to its role in breast and other cancers, Bcu631 is also expressed in other tissues, and thus is associated with other phenotypes, including other diseases and conditions. These, include, e.g., Retinitis pigmentosa 22, Microhydranencephaly (MHAC), Medullary Cystic Kidney Disease 2 (MCKD2), Paroxysmal kinesigenic choreoathetosis, Brody myopathy, Ceroid-lipofuscinosis neuronal-3, Glycogenosis, Familial Mitral valve prolapse, familial, susceptibility to Atopy, Convulsions, Arthrocutaneouveal granulomatosis. Nucleic acids of the present invention can be used as linkage markers, diagnostic targets, therapeutic targets, for any of the mentioned disorders, as well as any disorders or genes mapping in proximity to it.

Bcu738

Bcu738 (related to Hs.81281 and U79285) codes for an 87 amino acid polypeptide. The nucleotide and amino acid sequences of Bcu738 are shown in SEQ ID NOS 118-119. It contains a ribosomal protein S21 domain at about amino acid positions 10-62. The polypeptide coded for by Bcu738 exhibits sequence identity to U79285.

All or part of Bcu738 is located in genomic DNA represented by AL365403, BAC-ID RP5-835F16, and NT_{—}004698. The gene contains at least 2 exons. The present invention relates to any isolated introns and exons that are present in such clone. Such introns and exons can be routinely determined. Bcu738 maps to chromosomal band 1q12.

As indicated by its expression profile, Bcu738 is differentially-regulated in breast cancer. In addition to its role in breast and other cancers, Bcu738 is also expressed in other tissues, and thus is associated with other phenotypes, including other diseases and conditions. These, include, e.g., Phosphoglycerate dehydrogenase deficiency and Cone-rod dystrophy. Nucleic acids of the present invention can be used as linkage markers, diagnostic targets, therapeutic targets, for any of the mentioned disorders, as well as any disorders or genes mapping in proximity to it.

Bcu921

Bcu921 (related to AF220294) codes for a polypeptide having 190 amino acids. The nucleotide and amino acid sequences of Bcu921 are shown in SEQ ID NOS 127-128. It has internal repeats at amino acid positions 17-95 and 84-189. It is also related to the rat gene represented by AF220294 (Rsp29).

All or part of Bcu921 is located in genomic DNA represented by GenBank ID: AL031009 (1-418 bp), BAC-ID1:431H6 (1-418 bp), GenBank ID2: AL03171 (419-3034 bp), BAC-ID2: LA16-329F2, and Contig ID: NT_{—}010388. The gene contains at least 5 exons. The present invention relates to any isolated introns and exons that are present in such clone. Such introns and exons can be routinely determined. Bcu921 maps to chromosomal band 16p13.3.

As indicated by its expression profile, Bcu921 is differentially-regulated in breast cancer. In addition to its role in breast cancer, Bcu921 is also expressed in other tissues, and thus is associated with other phenotypes, including other diseases and conditions. These, include, e.g., Microphthalmia-cataract, Hemoglobin H-related mental retardation, Polycystic kidney disease, Myoclonic Epilepsy, and Microhydranencephaly (MHAC). Nucleic acids of the present invention can be used as linkage markers, diagnostic targets, therapeutic targets, for any of the mentioned disorders, as well as any disorders or genes mapping in proximity to it.

The present invention relates to the complete polynucleotide and polypeptide sequences disclosed herein, as well as fragments thereof. Useful fragments include those which are unique and which do not overlap any known gene (e.g., amino acids residues 1-105 of BCU0021 of SEQ ID NO 8), which overlap with a known sequence (e.g., amino acid residues 106-404 of BCU0021 of SEQ ID NO 8), which span alternative splice junctions (e.g., comprising amino acid residues 166-167 of BCU0156 of SEQ ID NO 22), which are unique to a public sequence as indicated in the Figures (e.g., amino acids 167-185 of XM_{—}108902 of SEQ ID NO 95), which span an alternative splice junction of a public sequence (e.g., 258-259 of XM_{—}108902 of SEQ ID NO 95), etc. Unique sequences can also be described as being specific for a gene because they are characteristic of the gene, but not related genes. The unique or specific sequences included polypeptide sequences, coding nucleotide sequences (e.g., as illustrated in the figures), and non-coding nucleotide sequences.

Below, for illustration, are some examples of polypeptides (included are the polynucleotides which encode them); however, the present invention includes all fragments, especially of the categories mentioned above are exemplified below.

BCU0021 (SEQ ID NO 7-8): polypeptides comprising, consisting of, or consisting essentially of about amino acids 1-105, polypeptide fragments thereof, and polynucleotides encoding said polypeptides;

BCU0067 (SEQ ID NO 9-10): polypeptides comprising, consisting of, or consisting essentially of about amino acids 1-112, polypeptide fragments thereof, and polynucleotides encoding said polypeptides;

BCU0156 (SEQ ID NO 21-22): polypeptides comprising, consisting of, or consisting essentially of about amino acids 240-279, 1759-1849, polypeptide fragments thereof, and polynucleotides encoding said polypeptides;

BCU0258 (SEQ ID NO 23-24): polypeptides comprising, consisting of, or consisting essentially of about amino acids 1-646, 860-1078, polypeptide fragments thereof, and polynucleotides encoding said polypeptides;

BCU0343 (SEQ ID NO 25-26): polypeptides comprising, consisting of, or consisting essentially of about amino acids 1-10, polypeptide fragments thereof, and polynucleotides encoding said polypeptides;

BCU0408 (SEQ ID NO 31-32): polypeptides comprising, consisting of, or consisting essentially of about amino acids 141-143, polypeptide fragments thereof, and polynucleotides encoding said polypeptides;

BCU0504 (SEQ ID NO 35-36): polypeptides comprising, consisting of, or consisting essentially of about amino acids 39-41, polypeptide fragments thereof, and polynucleotides encoding said polypeptides;

BCU0720 (SEQ ID NO 3940): polypeptides comprising, consisting of, or consisting essentially of about amino acids 1-504, polypeptide fragments thereof, and polynucleotides encoding said polypeptides;

BCU0721 (SEQ ID NO 41-42): polypeptides comprising, consisting of, or consisting essentially of about amino acids 27-35, 225, 526, 702, 707, polypeptide fragments thereof, and polynucleotides encoding said polypeptides;

BCU0730A (SEQ ID NO 43-44): polypeptides comprising, consisting of, or consisting essentially of about amino acids 27, 133-543, polypeptide fragments thereof, and polynucleotides encoding said polypeptides;

BCU0730B (SEQ ID NO 4546): polypeptides comprising, consisting of, or consisting essentially of about amino acids 27, 133-516, polypeptide fragments thereof, and polynucleotides encoding said polypeptides;

BCU0730C (SEQ ID NO 47-48): polypeptides comprising, consisting of, or consisting essentially of about amino acids 27, 133-538, polypeptide fragments thereof, and polynucleotides encoding said polypeptides;

BCU0730D (SEQ ID NO 49-50): polypeptides comprising, consisting of, or consisting essentially of about amino acids 27, 57-83, 160-574, polypeptide fragments thereof, and polynucleotides encoding said polypeptides;

BCU0770 (SEQ ID NO 51-52): polypeptides comprising, consisting of, or consisting essentially of about amino acids 1-37, 433463, polypeptide fragments thereof, and polynucleotides encoding said polypeptides;

BCU0840 (SEQ ID NO 53-54): polypeptides comprising, consisting of, or consisting essentially of about amino acids 220-221, 470-600, 639-655, polypeptide fragments thereof, and polynucleotides encoding said polypeptides;

BCU0947 (SEQ ID NO 61-62): polypeptides comprising, consisting of, or consisting essentially of about amino acids 1-1753, polypeptide fragments thereof, and polynucleotides encoding said polypeptides;

BCU1034 (SEQ ID NO 63-64): polypeptides comprising, consisting of, or consisting essentially of about amino acids 1-41, 42-59, 126, 195, polypeptide fragments thereof, and polynucleotides encoding said polypeptides;

BCU0988A (SEQ ID NO 83-84): polypeptides comprising, consisting of, or consisting essentially of about amino acids 1-59, 328-391, polypeptide fragments thereof, and polynucleotides encoding said polypeptides;

BCU0988B (SEQ ID NO 85-86): polypeptides comprising, consisting of, or consisting essentially of about amino acids 1-59, polypeptide fragments thereof, and polynucleotides encoding said polypeptides;

BCU0586 (SEQ ID NO 71-72): polypeptides comprising, consisting of, or consisting essentially of about amino acids 1-704, 705-711, polypeptide fragments thereof, and polynucleotides encoding said polypeptides;

BCU0715A (SEQ ID NO 73-74): polypeptides comprising, consisting of, or consisting essentially of about amino acids 1-113, 491, 675, polypeptide fragments thereof, and polynucleotides encoding said polypeptides;

BCU0715B (SEQ ID NO 75-76): polypeptides comprising, consisting of, or consisting essentially of about amino acids 91-92, polypeptide fragments thereof, and polynucleotides encoding said polypeptides;

BCU0715C (SEQ ID NO 77-78): polypeptides comprising, consisting of, or consisting essentially of about amino acids 104-105, polypeptide fragments thereof, and polynucleotides encoding said polypeptides;

BCU0205A (SEQ ID NO 79-80): polypeptides comprising, consisting of, or consisting essentially of about amino acids 1-85, polypeptide fragments thereof, and polynucleotides encoding said polypeptides;

BCU0205B (SEQ ID NO 81-82): polypeptides comprising, consisting of, or consisting essentially of about amino acids 1-79, 80-167, 168-945, polypeptide fragments thereof, and polynucleotides encoding said polypeptides;

BCU0518 (SEQ ID NO 87-88): polypeptides comprising, consisting of, or consisting essentially of about amino acids 1-35, polypeptide fragments thereof, and polynucleotides encoding said polypeptides.

Nucleic Acids

In accordance with the present invention, genes have been identified which are differentially expressed in breast cancer. These genes can be further divided into groups based on additional characteristics of their expression and the tissues in which they are expressed. For instance, genes can be further subdivided based on the stage and/or grade of the cancer in which they are expressed. Genes can also be grouped based on their penetrance in a breast cancer, e.g., expressed in all breast cancers examined, expressed in a certain percentage of breast cancers examined, etc. These groupings do not restrict or limit the use such genes in therapeutic, diagnostic, prognostic, etc., applications. For instance, a gene which is expressed in only some cancers (e.g., incompletely penetrant) may be useful in therapeutic applications to treat a subset of cancers. Similarly, a co-penetrant gene, or a gene which is expressed in breast and other normal tissues, may be useful as a therapeutic or diagnostic, even if its expression pattern is not highly breast cancer specific. Thus, the uses of the genes or their products are not limited by their patterns of expression.

By the phrase “differential expression,” it is meant that the levels of expression of a gene, as measured by its transcription or translation product, are different depending upon the specific cell-type or tissue (e.g., in an averaging assay that looks at a population of cells). There are no absolute amounts by which the gene expression levels must vary, as long as the differences are measurable.

The phrase “up-regulated” indicates that an mRNA transcript or other nucleic acid corresponding to a polynucleotide of the present invention is expressed in larger amounts in a cancer as compared to the same transcript expressed in normal cells from which the cancer was derived. In general, up-regulation can be assessed by any suitable method, including any of the nucleic acid detection and hybridization methods mentioned below, as well as polypeptide-based methods. Up-regulation also includes going from substantially no expression in a normal tissue, from detectable expression in a normal tissue, from significant expression in a normal tissue, to higher levels in the cancer.

The phrase “down-regulated” indicates that an mRNA transcript or other nucleic acid corresponding to a polynucleotide of the present invention is expressed in lower amounts in a cancer as compared to the same transcript expressed in normal cells from which the cancer was derived. A down-regulated gene can show no detectable expression, or any amount of expression which is less than the gene's expression in normal tissue.

Differential regulation can be determined by any suitable method, e.g., by comparing its abundance per gram of RNA (e.g., total RNA, polyadenylated mRNA, etc.) extracted from a breast tissue in comparison to the corresponding normal tissue. The normal tissue can be from the same or different individual or source. For convenience, it can be supplied as a separate component or in a kit in combination with probes and other reagents for detecting genes. The quantity by which a nucleic acid is differentially-regulated can be any value, e.g., about 10% more or less of normal expression, about 50% more or less of normal expression, 2-fold more or less, 5-fold more or less, 10-fold more or less, etc.

The amount of transcript can also be compared to a different gene in the same sample, especially a gene whose abundance is known and substantially no different in its expression between normal and cancer cells (e.g., a “control” gene). If represented as a ratio, with the quantity of differentially-regulated gene transcript in the numerator and the control gene transcript in the denominator, the ratio would be larger, e.g., in breast cancer than in a sample from normal breast tissue.

Differential-regulation can arise through a number of different mechanisms. The present invention is not bound by any specific way through which it occurs. Differential-regulation of a polynucleotide can occur, e.g., by modulating (1) transcriptional rate of the gene (e.g., increasing its rate, inducing or stimulating its transcription from a basal, low-level rate, etc.), (2) the post-transcriptional processing of RNA transcripts, (3) the transport of RNA from the nucleus into the cytoplasm, (4) the RNA nuclear and cytoplasmic turnover (e.g., by virtue of having higher stability or resistance to degradation), and combinations thereof. See, e.g., Tollervey and Caceras, Cell, 103:703-709, 2000.

A differentially-regulated polynucleotide is useful in a variety of different applications as described in greater details below. Because it is more abundant in cancer, it and its expression products can be used in a diagnostic test to assay for the presence of cancer, e.g., in tissue sections, in a biopsy sample, in total RNA, in lymph, in blood, etc. Differentially-regulated polynucleotides and polypeptides can be used individually, or in groups, to assess the cancer, e.g., to determine the specific type of cancer, its stage of development, the nature of the genetic defect, etc., or to assess the efficacy of a treatment modality. How to use polynucleotides in diagnostic and prognostic assays is discussed below. In addition, the polynucleotides and the polypeptides they encode, can serve as a target for therapy or drug discovery. A polypeptide, coded for by a differentially-regulated polynucleotide, which is displayed on the cell-surface, can be a target for immunotherapy to destroy, inhibit, etc., the diseased tissue. Differentially-regulated transcripts can also be used in drug discovery schemes to identify pharmacological agents which suppress, inhibit, etc., their differential-regulation, thereby preventing the phenotype associated with their expression. Thus, a differentially-regulated polynucleotide and its expression products of the present invention have significant applications in diagnostic, therapeutic, prognostic, drug development, and related areas. The expression patterns of the differentially expressed genes disclosed herein can be described as a “fingerprint” in that they are a distinctive pattern displayed by a cancer. Just as with a fingerprint, an expression pattern can be used as a unique identifier to characterize the status of a tissue sample. The list of genes represented SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147 provides an example of a cell expression profile for a breast cancer. It can be used as a point of reference to compare and characterize unknown samples and samples for which further information is sought. Tissue fingerprints can be used in many ways, e.g., to classify an unknown tissue as being a breast cancer, to determine the origin of a particular cancer (e.g., the origin of metastatic cells), to determine the presence of a cancer in a biopsy sample, to assess the efficacy of a cancer therapy in a human patient or a non-human animal model, to detect circulating cancer cells in blood or a lymph node biopsy, etc. While the expression profile of the complete gene set represented by SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, and 91, 115, 117, 118, 120, 123, 125, 127, 129, 131, 133, 135, 137, 139, 141, 143, 145, and/or 147 may be most informative, a fingerprint containing expression information from less than the full collection can be useful, as well. In the same way that an incomplete fingerprint may contain enough of the pattern of whorls, arches, loops, and ridges, to identify the individual, a cell expression fingerprint containing less than the full complement may be adequate to provide useful and unique identifying and other information about the sample. Moreover, cancer is a multifactorial disease, involving genetic aberrations in more than gene locus. This multifaceted nature may be reflected in different cell expression profiles associated with breast cancers arising in different individuals, in different locations in the same individual, or even within the same cancer locus. As a result, a complete match with a particular cell expression profile, as shown herein, is not necessary to classify a cancer as being of the same type or stage. Similarity to one cell expression profile, e.g., as compared to another, can be adequate to classify cancer types, grades, and stages.

A mammalian polynucleotide, or fragment thereof, of the present invention is a polynucleotide having a nucleotide sequence obtainable from a natural source. When the species name is used, e.g., human, it indicates that the polynucleotide or polypeptide is obtainable from a natural source. It therefore includes naturally-occurring normal It therefore includes naturally-occurring normal, naturally-occurring mutant, and naturally-occurring polymorphic alleles (e.g., SNPs), differentially-spliced transcripts, splice-variants, etc. By the term “naturally-occurring,” it is meant that the polynucleotide is obtainable from a natural source, e.g., animal tissue and cells, body fluids, tissue culture cells, forensic samples. Natural sources include, e.g., living cells obtained from tissues and whole organisms, tumors, cultured cell lines, including primary and immortalized cell lines. Naturally-occuring mutations can include deletions (e.g., a truncated amino- or carboxy-terminus), substitutions, inversions, or additions of nucleotide sequence. These genes can be detected and isolated by polynucleotide hybridization according to methods which one skilled in the art would know, e.g., as discussed below.

A polynucleotide according to the present invention can be obtained from a variety of different sources. It can be obtained from DNA or RNA, such as polyadenylated mRNA or total RNA, e.g., isolated from tissues, cells, or whole organism. The polynucleotide can be obtained directly from DNA or RNA, from a cDNA library, from a genomic library, etc. The polynucleotide can be obtained from a cell or tissue (e.g., from an embryonic or adult tissues) at a particular stage of development, having a desired genotype, phenotype, disease status, etc. The polynucleotides described in SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147 can be partial sequences that correspond to full-length, naturally-occurring transcripts. The present invention includes, as well, full-length polynucleotides that comprise these partial sequences, e.g., genomic DNAs and polynucleotides comprising a start and stop codon, a start codon and a polyA tail, a transcription start and a polyA tail, etc. These sequences can be obtained by any suitable method, e.g., using a partial sequence as a probe to select a full-length cDNA from a library containing full-length inserts. A polynucleotide which “codes without interruption” refers to a polynucleotide having a continuous open reading frame (“ORF”) as compared to an ORF which is interrupted by introns or other noncoding sequences.

Polynucleotides and polypeptides (including any part of a differentially regulated breast cancer gene) can be excluded as compositions from the present invention if, e.g., listed in a publicly available databases on the day this application was filed and/or disclosed in a patent application having an earlier filing or priority date than this application and/or conceived and/or reduced to practice earlier than a polynucleotide in this application.

As described herein, the phrase “an isolated polynucleotide which is SEQ ID NO,” or “an isolated polynucleotide which is selected from SEQ ID NO,” refers to an isolated nucleic acid molecule from which the recited sequence was derived (e.g., a cDNA derived from mRNA; cDNA derived from genomic DNA). Because of sequencing errors, typographical errors, etc., the actual naturally-occurring sequence may differ from a SEQ ID listed herein. Thus, the phrase indicates the specific molecule from which the sequence was derived, rather than a molecule having that exact recited nucleotide sequence, analogously to how a culture depository number refers to a specific cloned fragment in a cryotube. As explained in more detail below, a polynucleotide sequence of the invention can contain the complete sequence as shown in SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, 147, degenerate sequences thereof, anti-sense, muteins thereof, genes comprising said sequences, full-length cDNAs comprising said sequences, complete genomic sequences, fragments thereof, homologs, primers, nucleic acid molecules which hybridize thereto, derivatives thereof, etc.

Genomic

The present invention also relates genomic DNA from which the polynucleotides of the present invention can be derived. A genomic DNA coding for a human, mouse, or other mammalian polynucleotide, can be obtained routinely, for example, by screening a genomic library (e.g., a YAC library) with a polynucleotide of the present invention, or by searching nucleotide databases, such as GenBank and EMBL, for matches. Promoter and other regulatory regions (including both 5′ and 3′ regions, as well introns) can be identified upstream or downstream of coding and expressed RNAs, and assayed routinely for activity, e.g., by joining to a reporter gene (e.g., CAT, GFP, alkaline phosphatase, luciferase, galatosidase). A promoter obtained from a breast cancer gene can be used, e.g., in gene therapy to obtain cancer-specific expression of a heterologous gene (e.g., coding for a therapeutic product or cytotoxin). 5′ and 3′ sequences (including, UTRs and introns) can be used to modulate or regulate stability, transcription, and translation of nucleic acids, including the sequence to which is attached in nature, as well as heterologous nucleic acids.

Constructs

A polynucleotide of the present invention can comprise additional polynucleotide sequences, e.g., sequences to enhance expression, detection, uptake, cataloging, tagging, etc. A polynucleotide can include only coding sequence; a coding sequence and additional non-naturally occurring or heterologous coding sequence (e.g., sequences coding for leader, signal, secretory, targeting, enzymatic, fluorescent, antibiotic resistance, and other functional or diagnostic peptides); coding sequences and non-coding sequences, e.g., untranslated sequences at either a 5′ or 3′ end, or dispersed in the coding sequence, e.g., introns.

A polynucleotide according to the present invention also can comprise an expression control sequence operably linked to a polynucleotide as described above. The phrase “expression control sequence” means a polynucleotide sequence that regulates expression of a polypeptide coded for by a polynucleotide to which it is functionally (“operably”) linked. Expression can be regulated at the level of the mRNA or polypeptide. Thus, the expression control sequence includes mRNA-related elements and protein-related elements. Such elements include promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter. Expression control sequences can include an initiation codon and additional nucleotides to place a partial nucleotide sequence of the present invention in-frame in order to produce a polypeptide (e.g., pET vectors from Promega have been designed to permit a molecule to be inserted into all three reading frames to identify the one that results in polypeptide expression). Expression control sequences can be heterologous or endogenous to the normal gene.

A polynucleotide of the present invention can also comprise nucleic acid vector sequences, e.g., for cloning, expression, amplification, selection, etc. Any effective vector can be used. A vector is, e.g., a polynucleotide molecule which can replicate autonomously in a host cell, e.g., containing an origin of replication. Vectors can be useful to perform manipulations, to propagate, and/or obtain large quantities of the recombinant molecule in a desired host. A skilled worker can select a vector depending on the purpose desired, e.g., to propagate the recombinant molecule in bacteria, yeast, insect, or mammalian cells. The following vectors are provided by way of example. Bacterial: pQE70, pQE60, pQE-9 (Qiagen), pBS, pD10, Phagescript, phiX174, pBK Phagemid, pNH8A, pNH16a, pNH18Z, pNH46A (Stratagene); Bluescript KS+II (Stratagene); ptrc99a, pKK223-3, pKK233-3, pDR540, pRIT5 (Pharmacia). Eukaryotic: PWLNEO, pSV2CAT, pOG44, pXT1, pSG (Stratagene), pSVK3, PBPV, PMSG, pSVL (Pharmacia), pCR2.1/TOPO, pCRII/TOPO, pCR4/TOPO, pTrcHisB, pCMV6-XL4, etc. However, any other vector, e.g., plasmids, viruses, or parts thereof, may be used as long as they are replicable and viable in the desired host. The vector can also comprise sequences which enable it to replicate in the host whose genome is to be modified.

Hybridization

Polynucleotide hybridization, as discussed in more detail below, is useful in a variety of applications, including, in gene detection methods, for identifying mutations, for making mutations, to identify homologs in the same and different species, to identify related members of the same gene family, in diagnostic and prognostic assays, in therapeutic applications (e.g., where an antisense polynucleotide is used to inhibit expression), etc. The ability of two single-stranded polynucleotide preparations to hybridize together is a measure of their nucleotide sequence complementarity, e.g., base-pairing between nucleotides, such as A-T, G-C, etc. The invention thus also relates to polynucleotides, and their complements, which hybridize to a polynucleotide comprising a nucleotide sequence as set forth in SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147, and genomic sequences thereof. A nucleotide sequence hybridizing to the latter sequence will have a complementary polynucleotide strand, or act as a template for one in the presence of a polymerase (i.e., an appropriate polynucleotide synthesizing enzyme). The present invention includes both strands of polynucleotide, e.g., a sense strand and an anti-sense strand. Hybridization conditions can be chosen to select polynucleotides which have a desired amount of nucleotide complementarity with the nucleotide sequences set forth in SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, 147, and genomic sequences thereof. A polynucleotide capable of hybridizing to such sequence, preferably, possesses, e.g., about 70%, 75%, 80%, 85%, 87%, 90%, 92%, 95%, 97%, 99%, or 100% complementarity, between the sequences. The present invention particularly relates to polynucleotide sequences which hybridize to the nucleotide sequences set forth in SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, 147, or genomic sequences thereof, under low or high stringency conditions. These conditions can be used, e.g., to select corresponding homologs in non-human species.

Polynucleotides which hybridize to polynucleotides of the present invention can be selected in various ways. Filter-type blots (i.e., matrices containing polynucleotide, such as nitrocellulose), glass chips, and other matrices and substrates comprising polynucleotides (short or long) of interest, can be incubated in a prehybridization solution (e.g., 6×SSC, 0.5% SDS, 100 μg/ml denatured salmon sperm DNA, 5× Denhardt's solution, and 50% formamide), at 22-68° C., overnight, and then hybridized with a detectable polynucleotide probe under conditions appropriate to achieve the desired stringency. In general, when high homology or sequence identity is desired, a high temperature can be used (e.g., 65° C.). As the homology drops, lower washing temperatures are used. For salt concentrations, the lower the salt concentration, the higher the stringency. The length of the probe is another consideration. Very short probes (e.g., less than 100 base pairs) are washed at lower temperatures, even if the homology is high. With short probes, formamide can be omitted. See, e.g., Current Protocols in Molecular Biology, Chapter 6, Screening of Recombinant Libraries; Sambrook et al., Molecular Cloning, 1989, Chapter 9.

For instance, high stringency conditions can be achieved by incubating the blot overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1% SSC and 0.1% SDS for 30 min at 65° C.), i.e., selecting sequences having 95% or greater sequence identity.

Other non-limiting examples of high stringency conditions includes a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than 10%, less than 5% mismatch, etc., reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

Hybridization can also be based on a calculation of melting temperature (Tm) of the hybrid formed between the probe and its target, as described in Sambrook et al. Generally, the temperature Tm at which a short oligonucleotide (containing 18 nucleotides or fewer) will melt from its target sequence is given by the following equation: Tm=(number of A's and T's)×2° C.+(number of C's and G's)×4° C. For longer molecules, Tₘ=81.5+16.6 log₁₀[Na⁺]+0.41(% GC)-600/N where [Na⁺] is the molar concentration of sodium ions, % GC is the percentage of GC base pairs in the probe, and N is the length. Hybridization can be carried out at several degrees below this temperature to ensure that the probe and target can hybridize. Mismatches can be allowed for by lowering the temperature even further.

Stringent conditions can be selected to isolate sequences, and their complements, which have, e.g., at least about 90%, 95%, or 97%, nucleotide complementarity between the probe (e.g., a short polynucleotide of SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, 147, or genomic sequences thereof) and a target polynucleotide.

Other homologs of polynucleotides of the present invention can be obtained from mammalian and non-mammalian sources according to various methods. For example, hybridization with a polynucleotide can be employed to select homologs, e.g., as described in Sambrook et al., Molecular Cloning, Chapter 11, 1989. Such homologs can have varying amounts of nucleotide and amino acid sequence identity and similarity to such polynucleotides of the present invention. Mammalian organisms include, e.g., mice, rats, monkeys, pigs, cows, etc. Non-mammalian organisms include, e.g., vertebrates, invertebrates, zebra fish, chicken, Drosophila, C. elegans, Xenopus, yeast such as S. pombe, S. cerevisiae, roundworms, prokaryotes, plants, Arabidopsis, artemia, viruses, etc. The degree of nucleotide sequence identity between human and mouse can be about, e.g. 70% or more, 85% or more for open reading frames, etc.

Alignment

Alignments can be accomplished by using any effective algorithm. For pairwise alignments of DNA sequences, the methods described by Wilbur-Lipman (e.g., Wilbur and Lipman, Proc. Natl. Acad. Sci., 80:726-730, 1983) or Martinez/Needleman-Wunsch (e.g., Martinez, Nucleic Acid Res., 11:4629-4634, 1983) can be used. For instance, if the Martinez/Needleman-Wunsch DNA alignment is applied, the minimum match can be set at 9, gap penalty at 1.10, and gap length penalty at 0.33. The results can be calculated as a similarity index, equal to the sum of the matching residues divided by the sum of all residues and gap characters, and then multiplied by 100 to express as a percent. Similarity index for related genes at the nucleotide level in accordance with the present invention can be greater than 70%, 80%, 85%, 90%, 95%, 99%, or more. Pairs of protein sequences can be aligned by the Lipman-Pearson method (e.g., Lipman and Pearson, Science, 227:1435-1441, 1985) with k-tuple set at 2, gap penalty set at 4, and gap length penalty set at 12. Results can be expressed as percent similarity index, where related genes at the amino acid level in accordance with the present invention can be greater than 65%, 70%, 75%, 80%, 85%, 90%, 95%, 99%, or more. Various commercial and free sources of alignment programs are available, e.g., MegAlign by DNA Star, BLAST (National Center for Biotechnology Information), BCM (Baylor College of Medicine) Launcher, etc. BLAST can be used to calculate amino acid sequence identity, amino acid sequence homology, and nucleotide sequence identity. These calculations can be made along the entire length of each of the target sequences which are to be compared.

After two sequences have been aligned, a “percent sequence identity” can be determined. For these purposes, it is convenient to refer to a Reference Sequence and a Compared Sequence, where the Compared Sequence is compared to the Reference Sequence. Percent sequence identity can be determined according to the following formula: Percent Identity =100 [1−(C/R)], wherein C is the number of differences between the Reference Sequence and the Compared Sequence over the length of alignment between the Reference Sequence and the Compared Sequence where (i) each base or amino acid in the Reference Sequence that does not have a corresponding aligned base or amino acid in the Compared Sequence, (ii) each gap in the Reference Sequence, (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

Percent sequence identity can also be determined by other conventional methods, e.g., as described in Altschul et al., Bull. Math. Bio. 48: 603-616, 1986 and Henikoff and Henikoff, Proc. Natl. Acad. Sci. USA 89:10915-10919, 1992.

Specific Polynucleotide Probes

A polynucleotide of the present invention can comprise any continuous nucleotide sequence of SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147, sequences which share sequence identity thereto, or complements thereof. The term “probe” refers to any substance that can be used to detect, identify, isolate, etc., another substance. A polynucleotide probe is comprised of nucleic acid can be used to detect, identify, etc., other nucleic acids, such as DNA and RNA.

These polynucleotides can be of any desired size that is effective to achieve the specificity desired. For example, a probe can be from about 7 or 8 nucleotides to several thousand nucleotides, depending upon its use and purpose. For instance, a probe used as a primer PCR can be shorter than a probe used in an ordered array of polynucleotide probes. Probe sizes vary, and the invention is not limited in any way by their size, e.g., probes can be from about 7-2000 nucleotides, 7-1000, 8-700, 8-600, 8-500, 8-400, 8-300, 8-150, 8-100, 8-75, 7-50, 10-25, 14-16, at least about 8, at least about 10, at least about 15, at least about 25, etc. The polynucleotides can have non-naturally-occurring nucleotides, e.g., inosine, AZT, 3TC, etc. The polynucleotides can have 100% sequence identity or complementarity to a sequence of SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, 147, or it can have mismatches or nucleotide substitutions, e.g., 1, 2, 3, 4, or 5 substitutions. The probes can be single-stranded or double-stranded.

In accordance with the present invention, a polynucleotide can be present in a kit, where the kit includes, e.g., one or more polynucleotides, a desired buffer (e.g., phosphate, tris, etc.), detection compositions, RNA or cDNA from different tissues to be used as controls, libraries, etc. The polynucleotide can be labeled or unlabeled, with radioactive or non-radioactive labels as known in the art. Kits can comprise one or more pairs of polynucleotides for amplifying nucleic acids specific for differentially-regulated genes of the present invention, e.g., comprising a forward and reverse primer effective in PCR. These include both sense and anti-sense orientations. For instance, in PCR-based methods (such as RT-PCR), a pair of primers are typically used, one having a sense sequence and the other having an antisense sequence.

Another aspect of the present invention is a nucleotide sequence that is specific to, or for, a selective polynucleotide. The phrases “specific for” or “specific to” a polynucleotide have a functional meaning that the polynucleotide can be used to identify the presence of one or more target genes in a sample and distinguish them from non-target genes. It is specific in the sense that it can be used to detect polynucleotides above background noise (“non-specific binding”). A specific sequence is a defined order of nucleotides (or amino acid sequences, if it is a polypeptide sequence) which occurs in the polynucleotide, e.g., in the nucleotide sequences of SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, 147, and which is characteristic of that target sequence, and substantially no non-target sequences. A probe or mixture of probes can comprise a sequence or sequences that are specific to a plurality of target sequences, e.g., where the sequence is a consensus sequence, a functional domain, etc., e.g., capable of recognizing a family of related genes. Such sequences can be used as probes in any of the methods described herein or incorporated by reference. Both sense and antisense nucleotide sequences are included. A specific polynucleotide according to the present invention can be determined routinely.

A polynucleotide comprising a specific sequence can be used as a hybridization probe to identify the presence of, e.g., human or mouse polynucleotide, in a sample comprising a mixture of polynucleotides, e.g., on a Northern blot. Hybridization can be performed under high stringent conditions (see, above) to select polynucleotides (and their complements which can contain the coding sequence) having at least 90%, 95%, 99%, etc., identity (i.e., complementarity) to the probe, but less stringent conditions can also be used. A specific polynucleotide sequence can also be fused in-frame, at either its 5′ or 3′ end, to various nucleotide sequences as mentioned throughout the patent, including coding sequences for enzymes, detectable markers, GFP, etc, expression control sequences, etc.

A polynucleotide probe, especially one that is specific to a polynucleotide of the present invention, can be used in gene detection and hybridization methods as already described. In one embodiment, a specific polynucleotide probe can be used to detect whether a particular tissue or cell-type is present in a target sample. To carry out such a method, a selective polynucleotide can be chosen which is characteristic of the desired target tissue. Such polynucleotide is preferably chosen so that it is expressed or displayed in the target tissue, but not in other tissues which are present in the sample. For instance, if detection of breast cancer is desired, it may not matter whether the selective polynucleotide is expressed in other tissues, as long as it is not expressed in cells normally present in blood, e.g., peripheral blood mononuclear cells. Starting from the selective polynucleotide, a specific polynucleotide probe can be designed which hybridizes (if hybridization is the basis of the assay) under the hybridization conditions to the selective polynucleotide, whereby the presence of the selective polynucleotide can be determined.

Probes which are specific for polynucleotides of the present invention can also be prepared using involve transcription-based systems, e.g., incorporating an RNA polymerase promoter into a selective polynucleotide of the present invention, and then transcribing anti-sense RNA using the polynucleotide as a template. See, e.g., U.S. Pat. No. 5,545,522.

Polynucleotide Composition

A polynucleotide according to the present invention can comprise, e.g., DNA, RNA, synthetic polynucleotide, peptide polynucleotide, modified nucleotides, dsDNA, ssDNA, ssRNA, dsRNA, and mixtures thereof. A polynucleotide can be single- or double-stranded, triplex, DNA:RNA, duplexes, comprise hairpins, and other secondary structures, etc. Nucleotides comprising a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc.

Various modifications can be made to the polynucleotides, such as attaching detectable markers (avidin, biotin, radioactive elements, fluorescent tags and dyes, energy transfer labels, energy-emitting labels, binding partners, etc.) or moieties which improve hybridization, detection, and/or stability. The polynucleotides can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967, 5,476,925, and 5,478,893.

Polynucleotide according to the present invention can be labeled according to any desired method. The polynucleotide can be labeled using radioactive tracers such as ³²P, ³⁵S, ³H, or ¹⁴C, to mention some commonly used tracers. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

Nucleic Acid Detection Methods

Another aspect of the present invention relates to methods and processes for detecting differentially-regulated genes of the present invention. Detection methods have a variety of applications, including for diagnostic, prognostic, forensic, and research applications. To accomplish gene detection, a polynucleotide in accordance with the present invention can be used as a “probe.” The term “probe” or “polynucleotide probe” has its customary meaning in the art, e.g., a polynucleotide which is effective to identify (e.g., by hybridization), when used in an appropriate process, the presence of a target polynucleotide to which it is designed. Identification can involve simply determining presence or absence, or it can be quantitative, e.g., in assessing amounts of a gene or gene transcript present in a sample. Probes can be useful in a variety of ways, such as for diagnostic purposes, to identify homologs, and to detect, quantitate, or isolate a polynucleotide of the present invention in a test sample.

Assays can be utilized which permit quantification and/or presence/absence detection of a target nucleic acid in a sample. Assays can be performed at the single-cell level, or in a sample comprising many cells, where the assay is “averaging” expression over the entire collection of cells and tissue present in the sample. Any suitable assay format can be used, including, but not limited to, e.g., Southern blot analysis, Northern blot analysis, polymerase chain reaction (“PCR”) (e.g., Saiki et al., Science, 241:53, 1988; U.S. Pat. Nos. 4,683,195, 4,683,202, and 6,040,166; PCR Protocols: A Guide to Methods and Applications, Innis et al., eds., Academic Press, New York, 1990), reverse tmmscriptase polymerase chain reaction (“RT-PCR”), anchored PCR, rapid amplification of cDNA ends (“RACE”) (e.g., Schaefer in Gene Cloning and Analysis: Current Innovations, Pages 99-115, 1997), ligase chain reaction (“LCR”) (EP 320 308), one-sided PCR (Ohara et al., Proc. Natl. Acad. Sci., 86:5673-5677, 1989), indexing methods (e.g., U.S. Pat. No. 5,508,169), in situ hybridization, differential display (e.g., Liang et al., Nucl. Acid. Res., 21:3269-3275, 1993; U.S. Pat. Nos. 5,262,311, 5,599,672 and 5,965,409; WO97/18454; Prashar and Weissman, Proc. Natl. Acad. Sci., 93:659-663, and U.S. Pat. Nos. 6,010,850 and 5,712,126; Welsh et al., Nucleic Acid Res., 20:49654970, 1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), Qbeta Replicase (PCT/US87/00880), Strand Displacement Amplification (“SDA”), Repair Chain Reaction (“RCR”), nuclease protection assays, subtraction-based methods, Rapid-Scan™, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci., 88:7276-7280, 1991; U.S. Pat. Nos. 5,210,015 and 5,994,063), real-time fluorescence-based monitoring (e.g., U.S. Pat. No. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-309, 1996). Any method suitable for single cell analysis of gene or protein expression can be used, including in situ hybridization, immunocytochemistry, MACS, FACS, flow cytometry, etc. For single cell assays, expression products can be measured using antibodies, PCR, or other types of nucleic acid amplification (e.g., Brady et al., Methods Mol. & Cell. Biol. 2, 17-25, 1990; Eberwine et al., 1992, Proc. Natl. Acad. Sci., 89, 3010-3014, 1992; U.S. Pat. No. 5,723,290). These and other methods can be carried out conventionally, e.g., as described in the mentioned publications.

Many of such methods may require that the polynucleotide is labeled, or comprises a particular nucleotide type useful for detection. The present invention includes such modified polynucleotides that are necessary to carry out such methods. Thus, polynucleotides can be DNA, RNA, DNA:RNA hybrids, PNA, etc., and can comprise any modification or substituent which is effective to achieve detection.

Detection can be desirable for a variety of different purposes, including research, diagnostic, prognostic, and forensic. For diagnostic purposes, it may be desirable to identify the presence or quantity of a polynucleotide sequence in a sample, where the sample is obtained from tissue, cells, body fluids, etc. In a preferred method as described in more detail below, the present invention relates to a method of detecting a polynucleotide comprising, contacting a target polynucleotide in a test sample with a polynucleotide probe under conditions effective to achieve hybridization between the target and probe; and detecting hybridization.

Any test sample in which it is desired to identify a polynucleotide or polypeptide thereof can be used, including, e.g., blood, urine, saliva, stool (for extracting nucleic acid, see, e.g., U.S. Pat. No. 6,177,251), swabs comprising tissue, biopsied tissue, tissue sections, cultured cells, etc.

Detection can be accomplished in combination with polynucleotide probes for other genes, e.g., genes which are expressed in other disease states, tissues, cells, such as brain, heart, kidney, spleen, thymus, liver, stomach, small intestine, colon, muscle, lung, testis, placenta, pituitary, thyroid, skin, adrenal gland, pancreas, salivary gland, uterus, ovary, prostate gland, peripheral blood cells (T-cells, lymphocytes, etc.), embryo, normal breast fat, adult and embryonic stem cells, specific cell-types, such as endothelial, epithelial, myocytes, adipose, luminal epithelial, basoepithelial, myoepithelial, stromal cells, etc.

Polynucleotides can be used in wide range of methods and compositions, including for detecting, diagnosing, staging, grading, assessing, prognosticating, etc. diseases and disorders associated with differentially-regulated genes of the present invention, for monitoring or assessing therapeutic and/or preventative measures, in ordered arrays, etc. Any method of detecting genes and polynucleotides of SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147 can be used; certainly, the present invention is not to be limited how such methods are implemented.

Along these lines, the present invention relates to methods of detecting differentially-regulated genes described herein in a sample comprising nucleic acid. Such methods can comprise one or more the following steps in any effective order, e.g., contacting said sample with a polynucleotide probe under conditions effective for said probe to hybridize specifically to nucleic acid in said sample, and detecting the presence or absence of probe hybridized to nucleic acid in said sample, wherein said probe is a polynucleotide which is selected from SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, 147,a polynucleotide having, e.g., about 70%, 80%, 85%, 90%, 95%, 99%, or more sequence identity thereto, effective or specific fragments thereof, or complements thereto. The detection method can be applied to any sample, e.g., cultured primary, secondary, or established cell lines, tissue biopsy, blood, urine, stool, cerebral spinal fluid, and other bodily fluids, for any purpose.

Contacting the sample with probe can be carried out by any effective means in any effective environment. It can be accomplished in a solid, liquid, frozen, gaseous, amorphous, solidified, coagulated, colloid, etc., mixtures thereof, matrix. For instance, a probe in an aqueous medium can be contacted with a sample which is also in an aqueous medium, or which is affixed to a solid matrix, or vice-versa.

Generally, as used throughout the specification, the term “effective conditions” means, e.g., the particular milieu in which the desired effect is achieved. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including substrate, oxygen, carbon dioxide, etc.). When hybridization is the chosen means of achieving detection, the probe and sample can be combined such that the resulting conditions are functional for said probe to hybridize specifically to nucleic acid in said sample.

The phrase “hybridize specifically” indicates that the hybridization between single-stranded polynucleotides is based on nucleotide sequence complementarity. The effective conditions are selected such that the probe hybridizes to a preselected and/or definite target nucleic acid in the sample. For instance, if detection of a polynucleotide set forth in SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147 is desired, a probe can be selected which can hybridize to such target gene under high stringent conditions, without significant hybridization to other genes in the sample. To detect homologs of a polynucleotide set forth in SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147, the effective hybridization conditions can be less stringent, and/or the probe can comprise codon degeneracy, such that a homolog is detected in the sample.

As already mentioned, the methods can be carried out by any effective process, e.g., by Northern blot analysis, polymerase chain reaction (PCR), reverse transcriptase PCR, RACE PCR, in situ hybridization, etc., as indicated above. When PCR based techniques are used, two or more probes are generally used. One probe can be specific for a defined sequence which is characteristic of a selective polynucleotide, but the other probe can be specific for the selective polynucleotide, or specific for a more general sequence, e.g., a sequence such as polyA which is characteristic of mRNA, a sequence which is specific for a promoter, ribosome binding site, or other transcriptional features, a consensus sequence (e.g., representing a functional domain). For the former aspects, 5′ and 3′ probes (e.g., polyA, Kozak, etc.) are preferred which are capable of specifically hybridizing to the ends of transcripts. When PCR is utilized, the probes can also be referred to as “primers” in that they can prime a DNA polymerase reaction.

In addition to testing for the presence or absence of polynucleotides, the present invention also relates to determining the amounts at which polynucleotides of the present invention are expressed in sample and determining the differential expression of such polynucleotides in samples. Such methods can involve substantially the same steps as described above for presence/absence detection, e.g., contacting with probe, hybridizing, and detecting hybridized probe, but using more quantitative methods and/or comparisons to standards.

The amount of hybridization between the probe and target can be determined by any suitable methods, e.g., PCR, RT-PCR, RACE PCR, Northern blot, polynucleotide microarrays, Rapid-Scan, etc., and includes both quantitative and qualitative measurements. For further details, see the hybridization methods described above and below. Determining by such hybridization whether the target is differentially expressed (e.g., up-regulated or down-regulated) in the sample can also be accomplished by any effective means. For instance, the target's expression pattern in the sample can be compared to its pattern in a known standard, such as in a normal tissue, or it can be compared to another gene in the same sample. When a second sample is utilized for the comparison, it can be a sample of normal tissue that is known not to contain diseased cells. The comparison can be performed on samples which contain the same amount of RNA (such as polyadenylated RNA or total RNA), or, on RNA extracted from the same amounts of starting tissue. Such a second sample can also be referred to as a control or standard. Hybridization can also be compared to a second target in the same tissue sample. Experiments can be performed that determine a ratio between the target nucleic acid and a second nucleic acid (a standard or control), e.g., in a normal tissue. When the ratio between the target and control are substantially the same in a normal and sample, the sample is determined or diagnosed not to contain cells. However, if the ratio is different between the normal and sample tissues, the sample is determined to contain cancer cells. The approaches can be combined, and one or more second samples, or second targets can be used. Any second target nucleic acid can be used as a comparison, including “housekeeping” genes, such as beta-actin, alcohol dehydrogenase, or any other gene whose expression does not vary depending upon the disease status of the cell.

Methods of Identifying Polymorphisms, Mutations, Etc., of a Differentially-Regulated Gene

Polynucleotides of the present invention can also be utilized to identify mutant alleles, SNPs, gene rearrangements and modifications, and other polymorphisms of the wild-type gene. Mutant alleles, polymorphisms, SNPs, etc., can be identified and isolated from cancers that are known, or suspected to have, a genetic component. Identification of such genes can be carried out routinely (see, above for more guidance), e.g., using PCR, hybridization techniques, direct sequencing, mismatch reactions (see, e.g., above), RFLP analysis, SSCP (e.g., Orita et al., Proc. Natl. Acad. Sci., 86:2766, 1992), etc., where a polynucleotide having a sequence selected from SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147 is used as a probe. The selected mutant alleles, SNPs, polymorphisms, etc., can be used diagnostically to determine whether a subject has, or is susceptible to a disorder associated with a differentially-regulated gene, as well as to design therapies and predict the outcome of the disorder. Methods involve, e.g., diagnosing a disorder associated with a differentially-regulated gene or determining susceptibility to a disorder, comprising, detecting the presence of a mutation in a gene represented by a polynucleotide selected from SEQ D NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147. The detecting can be carried out by any effective method, e.g., obtaining cells from a subject, determining the gene sequence or structure of a target gene (using, e.g., mRNA, cDNA, genomic DNA, etc), comparing the sequence or structure of the target gene to the structure of the normal gene, whereby a difference in sequence or structure indicates a mutation in the gene in the subject. Polynucleotides can also be used to test for mutations, SNPs, polymorphisms, etc., e.g., using mismatch DNA repair technology as described in U.S. Pat. No. 5,683,877; U.S. Pat. No. 5,656,430; Wu et al., Proc. Natl. Acad. Sci., 89:8779-8783, 1992.

The present invention also relates to methods of detecting polymorphisms in a differentially-regulated gene, comprising, e.g., comparing the structure of: genomic DNA comprising all or part of said gene, mRNA comprising all or part of said gene, cDNA comprising all or part of said gene, or a polypeptide comprising all or part of said gene, with the structure of said gene as set forth herein. The methods can be carried out on a sample from any source, e.g., cells, tissues, body fluids, blood, urine, stool, hair, egg, sperm, etc.

These methods can be implemented in many different ways. For example, “comparing the structure” steps include, but are not limited to, comparing restriction maps, nucleotide sequences, amino acid sequences, RFLPs, DNAse sites, DNA methylation fingerprints (e.g., U.S. Pat. No. 6,214,556), protein cleavage sites, molecular weights, electrophoretic mobilities, charges, ion mobility, etc., between a standard gene and a test gene. The term “structure” can refer to any physical characteristics or configurations which can be used to distinguish between nucleic acids and polypeptides. The methods and instruments used to accomplish the comparing step depends upon the physical characteristics which are to be compared. Thus, various techniques are contemplated, including, e.g., sequencing machines (both amino acid and polynucleotide), electrophoresis, mass spectrometer (U.S. Pat. Nos. 6,093,541, 6,002,127), liquid chromatography, HPLC, etc.

To carry out such methods, “all or part” of the gene or polypeptide can be compared. For example, if nucleotide sequencing is utilized, the entire gene can be sequenced, including promoter, introns, and exons, or only parts of it can be sequenced and compared, e.g., exon 1, exon 2, etc.

Mutagenesis

Mutated polynucleotide sequences of the present invention are useful for various purposes, e.g., to create mutations of the polypeptides they encode, to identify functional regions of genomic DNA, to produce probes for screening libraries, etc. Mutagenesis can be carried out routinely according to any effective method, e.g., oligonucleotide-directed (Smith, M., Ann. Rev. Genet. 19:423-463, 1985), degenerate oligonucleotide-directed (Hill et al., Method Enzymology, 155:558-568, 1987), region-specific (Myers et al., Science, 229:242-246, 1985; Derbyshire et al., Gene, 46:145, 1986; Ner et al., DNA, 7:127, 1988), linker-scanning (McKnight and Kingsbury, Science, 217:316-324, 1982), directed using PCR, recursive ensemble mutagenesis (Arkin and Yourvan, Proc. Natl. Acad. Sci., 89:7811-7815, 1992), random mutagenesis (e.g., U.S. Pat. Nos. 5,096,815; 5,198,346; and 5,223,409), site-directed mutagenesis (e.g., Walder et al., Gene, 42:133, 1986; Bauer et al., Gene, 37:73, 1985; Craik, Bio Techniques, January 1985, 12-19; Smith et al., Genetic Engineering: Principles and Methods, Plenum Press, 1981), phage display (e.g., Lowman et al., Biochem. 30:10832-10837, 1991; Ladner et al., U.S. Pat. No. 5,223,409; Huse, WIPO Publication WO 92/06204), etc. Desired sequences can also be produced by the assembly of target sequences using mutually priming oligonucleotides (Uhlmann, Gene, 71:2940, 1988). For directed mutagenesis methods, analysis of the three-dimensional structure of the polypeptide can be used to guide and facilitate making mutants which effect polypeptide activity. Sites of substrate-enzyme interaction or other biological activities can also be determined by analysis of crystal structure as determined by such techniques as nuclear magnetic resonance, crystallography or photoaffinity labeling. See, for example, de Vos et al., Science 255:306-312, 1992; Smith et al., J. Mol. Biol. 224:899-904, 1992; Wlodaver et al., FEBS Lett. 309:59-64, 1992.

In addition, libraries of differentially-regulated genes and fragments thereof can be used for screening and selection of gene variants. For instance, a library of coding sequences can be generated by treating a double-stranded DNA with a nuclease under conditions where the nicking occurs, e.g., only once per molecule, denaturing the double-stranded DNA, renaturing it to for double-stranded DNA that can include sense/antisense pairs from different nicked products, removing single-stranded portions from reformed duplexes by treatment with S1 nuclease, and ligating the resulting DNAs into an expression vector. By this method, expression libraries can be made comprising “mutagenized” differentially-regulated genes. The entire coding sequence or parts thereof can be used.

Polynucleotide Expression, Polypeptides Produced Thereby, and Specific-Binding Partners Thereto

A polynucleotide according to the present invention can be expressed in a variety of different systems, in vitro and in vivo, according to the desired purpose. For example, a polynucleotide can be inserted into an expression vector, introduced into a desired host, and cultured under conditions effective to achieve expression of a polypeptide coded for by the polynucleotide, to search for specific binding partners. Effective conditions include any culture conditions which are suitable for achieving production of the polypeptide by the host cell, including effective temperatures, pH, medium, additives to the media in which the host cell is cultured (e.g., additives which amplify or induce expression such as butyrate, or methotrexate if the coding polynucleotide is adjacent to a dhfr gene), cycloheximide, cell densities, culture dishes, etc. A polynucleotide can be introduced into the cell by any effective method including, e.g., naked DNA, calcium phosphate precipitation, electroporation, injection, DEAE-Dextran mediated transfection, fusion with liposomes, association with agents which enhance its uptake into cells, viral transfection. A cell into which a polynucleotide of the present invention has been introduced is a transformed host cell. The polynucleotide can be extrachromosomal or integrated into a chromosome(s) of the host cell. It can be stable or transient. An expression vector is selected for its compatibility with the host cell. Host cells include, mammalian cells, e.g., COS, CV1, BHK, CHO, HeLa, LTK, NIH 3T3, ZR-75-1 (ATCC CRL-1500), ZR-75-30 (ATCC CRL-1504), UACC-812 (ATCC CRL-1897), UACC-893 (ATCC CRL-1902), HCC38 (ATCC CRL-2314), HCC70 (CRL-2315), and other HCC cell lines (e.g., as deposited with the ATCC), AU565 (ATCC CRL-2351), Hs 496.T (ATCC CRL-7303), Hs 748.T (ATCC CRL-7486), SW527 (ATCC CRL-7940), 184A1(ATCC CRL-8798), MCF cell lines (e.g., 10A and others deposited with the ATCC), MDA-MB-134-VI (ATCC HTB-23 and other MDA cell lines), SK-BR-3 (ATCC HTB-30), ME-180 (ATCC HTB-33), Hs 578Bst (ATCC HTB-125), Hs 578T (ATCC HTB-126), T-47D (ATCC HTB-133), insect cells, such as Sf9 (S. frugipeda) and Drosophila, bacteria, such as E. coli, Streptococcus, bacillus, yeast, such as Sacharomyces, S. cerevisiae, fungal cells, plant cells, embryonic or adult stem cells (e.g., mammalian, such as mouse or human).

Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type specific elements, or sequences which allow selective or specific cell expression. Promoters that can be used to drive its expression, include, e.g., the endogenous promoter, MMTV, SV40, trp, lac, tac, or T7 promoters for bacterial hosts; or alpha factor, alcohol oxidase, or PGH promoters for yeast. RNA promoters can be used to produced RNA transcripts, such as T7 or SP6. See, e.g., Melton et al., Polynucleotide Res., 12(18):7035-7056, 1984; Dunn and Studier. J. Mol. Bio., 166:477-435, 1984; U.S. Pat. No. 5,891,636; Studier et al., Gene Expression Technology, Methods in Enzymology, 85:60-89, 1987. In addition, as discussed above, translational signals (including in-frame insertions) can be included.

When a polynucleotide is expressed as a heterologous gene in a transfected cell line, the gene is introduced into a cell as described above, under effective conditions in which the gene is expressed. The term “heterologous” means that the gene has been introduced into the cell line by the “hand-of-man.” Introduction of a gene into a cell line is discussed above. The transfected (or transformed) cell expressing the gene can be lysed or the cell line can be used intact.

For expression and other purposes, a polynucleotide can contain codons found in a naturally-occurring gene, transcript, or cDNA, for example, e.g., as set forth in SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147, or it can contain degenerate codons coding for the same amino acid sequences. For instance, it may be desirable to change the codons in the sequence to optimize the sequence for expression in a desired host. See, e.g., U.S. Pat. Nos. 5,567,600 and 5,567,862.

A polypeptide according to the present invention can be recovered from natural sources, transformed host cells (culture medium or cells) according to the usual methods, including, detergent extraction (e.g., non-ionic detergent, Triton X-100, CHAPS, octylglucoside, Igepal CA-630), ammonium sulfate or ethanol precipitation, acid extraction, anion or cation exchange chromatography, phosphocellulose chromatography, hydrophobic interaction chromatography, hydroxyapatite chromatography, lectin chromatography, gel electrophoresis. Protein refolding steps can be used, as necessary, in completing the configuration of the mature protein. Finally, high performance liquid chromatography (HPLC) can be employed for purification steps. Another approach is express the polypeptide recombinantly with an affinity tag (Flag epitope, HA epitope, myc epitope, 6×His, maltose binding protein, chitinase, etc) and then purify by anti-tag antibody-conjugated affinity chromatography.

The present invention also relates to polypeptides corresponding to the genes of the present invention, e.g., an isolated human polypeptide comprising or having the amino acid sequence set forth in [SEQ ID NO], an isolated human polypeptide comprising an amino acid sequence having 90%, 92%, 95%, 97%, 98%, or more amino acid sequence identity to the amino acid sequence set forth in [SEQ ID NO], and optionally having one or more of its specific activities. Fragments specific to these polypeptides can be used, e.g., to produce antibodies or other immune responses, as competitors to activity, competitors to ligands and other binding-partners, etc. These fragments can be referred to as being “specific for” the polypeptide. The latter phrase, as already defined, indicates that the peptides are characteristic of it, and that the defined sequences are substantially absent from all other protein types. Such polypeptides can be of any size which is necessary to confer specificity, e.g., 5, 8, 10, 12, 15, 20, etc.

The present invention also relates to specific-binding partners. These include antibodies which are specific for polypeptides encoded by polynucleotides of the present invention, as well as other binding-partners which interact with polynucleotides and polypeptides of the present invention. Protein-protein interactions between polypeptides of the present invention and other polypeptides and binding partners can be identified using any suitable methods, e.g., protein binding assays (e.g., filtration assays, chromatography, etc.), yeast two-hybrid system (Fields and Song, Nature, 340: 245-247, 1989), protein arrays, gel-shift assays, FRET (fluorescence resonance energy transfer) assays, etc. Nucleic acid interactions (e.g., protein-DNA or protein-RNA) can be assessed using gel-shift assays, e.g., as carried out in U.S. Pat. Nos. 6,333,407 and 5,789,538.

The present invention also relates to antibodies, and other specific-binding partners, which are specific for polypeptides encoded by polynucleotides of the present invention. Antibodies, e.g., polyclonal, monoclonal, recombinant, chimeric, humanized, single-chain, Fab, and fragments thereof, can be prepared according to any desired method. See, also, screening recombinant immunoglobulin libraries (e.g., Orlandi et al., Proc. Natl. Acad. Sci., 86:3833-3837, 1989; Huse et al., Science, 256:1275-1281, 1989); in vitro stimulation of lymphocyte populations; Winter and Milstein, Nature, 349: 293-299, 1991. The antibodies can be IgM, IgG, subtypes, IgG2a, IgG 1, etc. Antibodies, and immune responses, can also be generated by administering naked DNA See, e.g., U.S. Pat. Nos. 5,703,055; 5,589,466; 5,580,859. Antibodies can be used from any source, including, goat, rabbit, mouse, chicken (e.g., IgY; see, Duan, WO/029444 for methods of making antibodies in avian hosts, and harvesting the antibodies from the eggs). An antibody specific for a polypeptide means that the antibody recognizes a defined sequence of amino acids within or including the polypeptide. Other specific binding partners include, e.g., aptamers and PNA, can be prepared against specific epitopes or domains of differentially regulated genes.

The preparation of polyclonal antibodies is well-known to those skilled in the art. See, for example, Green et al., Production of Polyclonal Antisera, in IMMUNOCHEMICAL PROTOCOLS (Manson, ed.), pages 1-5 (Humana Press 1992); Coligan et al., Production of Polyclonal Antisera in Rabbits, Rats, Mice and Hamsters, in CURRENT PROTOCOLS IN IMMUNOLOGY, section 2.4.1 (1992). The preparation of monoclonal antibodies likewise is conventional. See, for example, Kohler & Milstein, Nature 256:495 (1975); Coligan et al., sections 2.5.1-2.6.7; and Harlow et al., ANTIBODIES: A LABORATORY MANUAL, page 726 (Cold Spring Harbor Pub. 1988).

Antibodies can also be humanized, e.g., where they are to be used therapeutically. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989), which is hereby incorporated in its entirety by reference. Techniques for producing humanized monoclonal antibodies are described, for example, in U.S. Pat. No. 6,054,297, Jones et al., Nature 321:522 (1986); Riechmann et al., Nature 332:323 (1988); Verhoeyen et al., Science 239:1534 (1988); Carter et al., Proc. Nat'l Acad. Sci. USA 89:4285 (1992); Sandhu, Crit. Rev. Biotech. 12: 437 (1992); and Singer et al., J. Immunol. 150: 2844 (1993).

Antibodies of the invention also may be derived from human antibody fragments isolated from a combinatorial immunoglobulin library. See, for example, Barbas et al., METHODS: A COMPANION TO METHODS IN ENZYMOLOGY, VOL. 2, page 119 (1991); Winter et al., Ann. Rev. Immunol. 12: 433 (1994). Cloning and expression vectors that are useful for producing a human immunoglobulin phage library can be obtained commercially, for example, from STRATAGENE Cloning Systems (La Jolla, Calif.).

In addition, antibodies of the present invention may be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens and can be used to produce human antibody-secreting hybridomas. Methods for obtaining human antibodies from transgenic mice are described, e.g., in Green et al., Nature Genet. 7:13 (1994); Lonberg et al., Nature 368:856 (1994); and Taylor et al., Int. Immunol. 6:579 (1994).

Antibody fragments of the present invention can be prepared by proteolytic hydrolysis of the antibody or by expression in E. coli of nucleic acid encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′).sub.2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. No. 4,036,945 and No. 4,331,647, and references contained therein. These patents are hereby incorporated in their entireties by reference. See also Nisoiihoff et al., Arch. Biochem. Biophys. 89:230 (1960); Porter, Biochem. J. 73:119 (1959); Edelman et al, METHODS IN ENZYMOLOGY, VOL. 1, page 422 (Academic Press 1967); and Coligan et al. at sections 2.8.1-2.8.10 and 2.10.1-2.10.4.

Other methods of cleaving antibodies, such as separation of heavy chains to form monovalent light-heavy chain fragments, further cleavage of fragments, or other enzymatic, chemical, or genetic techniques can also be used. For example, Fv fragments comprise an association of V.sub.H and V.sub.L chains. This association may be noncovalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde. See, e.g., Sandhu, supra. Preferably, the Fv fragments comprise V.sub.H and V.sub.L chains connected by a peptide linker. These single-chain antigen binding proteins (sFv) are prepared by constructing a structural gene comprising nucleic acid sequences encoding the V.sub.H and V.sub.L domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow et al., METHODS: A COMPANION TO METHODS IN ENZYMOLOGY, VOL. 2, page 97 (1991); Bird et al., Science 242:423-426 (1988); Ladneret al., U.S. Pat. No. 4,946,778; Pack et al., Bio/Technology 11: 1271-77 (1993); and Sandhu, supra.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al., METHODS: A COMPANION TO METHODS IN ENZYMOLOGY, VOL. 2, page 106 (1991).

The term “antibody” as used herein includes intact molecules as well as fragments thereof, such as Fab, F(ab′)₂, and Fv which are capable of binding to an epitopic determinant present in Bin1 polypeptide. Such antibody fragments retain some ability to selectively bind with its antigen or receptor. The term “epitope” refers to an antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics. Antibodies can be prepared against specific epitopes or polypeptide domains.

Antibodies which bind to a differentially-regulated polypeptide of the present invention can be prepared using an intact polypeptide or fragments containing small peptides of interest as the immunizing antigen. For example, it may be desirable to produce antibodies that specifically bind to the N- or C-terminal domains of said polypeptide. The polypeptide or peptide used to immunize an animal which is derived from translated cDNA or chemically synthesized which can be conjugated to a carrier protein, if desired. Such commonly used carriers which are chemically coupled to the immunizing peptide include keyhole limpet hemocyanin (KLH), thyroglobulin, bovine serum albumin (BSA), and tetanus toxoid.

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See for example, Coligan, et al., Unit 9, Current Protocols in Immunology, Wiley Interscience, 1994, incorporated by reference).

Anti-idiotype technology can also be used to produce invention monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

Methods of Detecting Polypeptides

Polypeptides coded for by a differentially-regulated gene of the present invention can be detected, visualized, determined, quantitated, etc. according to any effective method useful methods include, e.g., but are not limited to, immunoassays, RIA (radioimmunassay), ELISA, (enzyme-linked-immunosorbent assay), immunoflourescence, flow cytometry, histology, electron microscopy, light microscopy, in situ assays, immunoprecipitation, Western blot, etc.

Immunoassays may be carried in liquid or on biological support. For instance, a sample (e.g., blood, stool, urine, cells, tissue, body fluids, etc.) can be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled differentially-regulated gene specific antibody. The solid phase support can then be washed with a buffer a second time to remove unbound antibody. The amount of bound label on solid support may then be detected by conventional means.

A “solid phase support or carrier” includes any support capable of binding an antigen, antibody, or other specific binding partner. Supports or carriers include glass, polystyrene, polypropylene, polyethylene, dextran, nylon, amylases, natural and modified celluloses, polyacrylamides, and magnetite. A support material can have any structural or physical configuration. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads

One of the many ways in which gene peptide-specific antibody can be detectably labeled is by linking it to an enzyme and using it in an enzyme immunoassay (EIA). See, e.g., Voller, A., “The Enzyme Linked Immunosorbent Assay (ELISA),” 1978, Diagnostic Horizons 2, 1-7, Microbiological Associates Quarterly Publication, Walkersville, Md.); Voller, A. et al., 1978, J. Clin. Pathol. 31, 507-520; Butler, J. E., 1981, Meth. Enzymol. 73, 482-523; Maggio, E. (ed.), 1980, Enzyme Imunoassay, CRC Press, Boca Raton, Fla. The enzyme which is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, .alpha.-glycerophosphate, dehydrogenase, triose phosphate isomerase, horseradish peroxidase, alkaline phosphatase, asparaginase, glucose oxidase, .beta.-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect differentially-regulated peptides through the use of a radioimmunoassay (RIA). See, e.g., Weintraub, B., Principles of Radioimmunoassays, Seventh Training Course on Radioligand Assay Techniques, The Endocrine Society, March, 1986. The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as those in the lanthamide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction.

Examples of useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Tissue and Disease

The normal female breast comprises ducts and lobuloalveolar structures surrounded by basement membranes and collagenous stroma with fibroblasts, vessels, and fat. The basic unit of function in the breast are the lobuloalveolar structures which produce the milk secretions. Each lobule drains into a lactiferous duct that empties into a lactiferous sinus beneath the nipple. The ducts are lined with epithelial cells, containing few mitochondria and sparse endoplasmic reticulum. The lobules contain luminal epithelial cells, basal epithelial cells, and myoepithelial cells. The basal and epithelial cells are sometimes grouped together. The luminal cells can be differentiated immuno-histochemically from the myoepithelial cells by their expression of keratins. The luminal cells stain with antibodies to keratin 5/6; the myoepithelial cells stains with antibodies against keratin 8/18. In addition to the presence of these cells types in the breast, there are endothelial cells associated with blood vessels, stromal cells that surround the lobular structures, adipose cells, and blood cells, such as T-lymphocytes and macrophages.

Breast carcinoma can be classified into two basic types, noninvasive (non-infiltrating) and invasive. Noninvasive carcinoma includes, e.g., intraductal carcinoma (also known as ductal carcinoma in situ or “DCIS”), intraductal papillary carcinoma, and lobular carcinoma in situ. Invasive carcinoma includes, e.g., invasive ductal carcinoma (“IDC”), invasive lobular carcinoma, medullary carcinoma, colloid carcinoma (mucinous carcinoma), Paget's disease, tubular carcinoma, adenoid cystic carcinoma, invasive comedocarcinoma, apocrine carcinoma, and invasive papillary carcinoma. See, also, Cancer, Principles and Practice of Oncology, DeVita et al., ed., J.B. Lippincott Company, 1982, Pages 914-922. The different cancers can generally be distinguished histologically from each other.

Over 90% of breast cancers arise in the ducts. As long as it remains with the ductal basement membranes, it is classified as a non-infiltrating or non-invasive carcinoma. DCIS is a common example. An invasive or infiltrating carcinoma shows a marked increase in dense fibrous tissue stroma, giving the tissue a hard consistency. IDC is one of the more common types of an invasive carcinoma. Frequently, an infiltrating carcinoma becomes invaded with blood and lymphatic vessels as it increases in size and malignancy. The tumor cells fill the ducts, plugging them, and invade the surrounding stroma. For general description of breast pathology, see, e.g., Robins Pathological Basis of Disease, Cotran et al., 4^{th} Edition, W. B. Saunders Company, 1989, Chapter 25.

The progression of a cancer, from its origin to a full-blown malignancy, is the subject of intense study. Hyperplasia is generally believed to precede at least some cancers, but not all hyperplasia leads to cancer, and the relationship between the two is not well understood. One hallmark of a hyperplasia that leads to cancer may be the occurrence of genomic instability, and other factors which lead to uncoupling of the cell cycle.

Intraepithelial neoplasia is one of the first detectable signs of a breast cancer, characterized by its confinement to the duct epithelia. It can also be referred to as preinvasive neoplasia, precancer, dysplasia, or CIS. See, e.g., Boone et al., Proc. Soc. Exp. Biol. Med., 216:151-165, 1997. An intraepithelial neoplasia generally consists of multiple foci of an abnormal clonal expansion of neoplastic cells. The development of the neoplasia is manifested by an increasing size of the lesion and a greater degree of cytonuclear morphological aberration, as it progresses from low grade to high grade. See, e.g., Bacus et al., Cancer Epid. Biom. Prevent., 8:1087-1094, 1999. An early grade can be referred to as an intraductal proliferation (IDP). More advanced, pre-invasive lesions are DCIS and LCIS (lobular carcinoma in situ). It is believed that DCIS and LCIS are precursor lesions of invasive breast cancer, such as IDC. See, e.g., Buerger et al., Mol. Pathol., 53:118-121, 2000.

Breast cancers can be both staged and graded. Stage is based on the tumor and size and whether the lymph nodes are involved with the tumor. Tumor grade refers to the tumor cells' appearance under the microscope, and how closely it resembles normal tissue of the same type. If the tumor cells look normal, then it can be termed “low grade.” High grade cells look markedly different from normal cells. High grade tumors tend to behave more aggressively than lower grade. An “ungraded” cancer indicates that the gene expression profile as described herein indicates that it has an expression profile of group DI genes.

The most widely used clinical staging system for breast cancer is one adopted by the UICC (International Union against Cancer). This system incorporates the TNM (t, tumor; N, nodes; M, metastases) classification using tumor size, involvement of the chest wall and skin, inflammatory cancer, involvement of nodes, evidence of metastases. See, e.g., Sainsbury et al., BMJ, 321:745-750, 2000. Other staging and grading systems can also be used, e.g., Bloom and Richardson grade (British J Cancer, 11:359-377, 1957), Columbia Clinical Classification (CCC), Van Nuys (VN), etc. Grading systems have also been devised based on image analysis of neoplastic and normal cells. Bacus et al. (Cancer Epid. Biom. Prevent., 8:1087-1094, 1999) have described an image morphometric nuclear grading system for intraepitheliam neoplastic lesions, such as DCIS, which provides objective criteria to assess tumor grade. See, also, Schwartz, Human Pathol., 28:1798-1802, 1997, for a grading system for DCIS. FISH has also been used to diagnose cancers based on chromosomal aberrations. See, e.g., Komoike et al., Breast Cancer, 7:332-336, 2000.

Various genetic bases for breast cancer have begun to be identified. For instance, BRCA 1, BRCA2, ATM, PTEN/MMAC1 (e.g., Ali et al., J Natl Cancer Inst., 91:1922-1932, 1999), MLH2, MSH2, TP53 (e.g., Done et al., Cancer Res., 58:785-789, 1998), and STK11 are associated with a higher risk of cancer. Other genes involved in breast cancer include, e.g., myc, cyclin D1 (e.g., Weinstat-Saslow et al., Nature Med., 1:1257-1260, 1995), and c-erb-B2.

Grading, Staging, Comparing, Assessing, Methods and Compositions

The present invention also relates to methods and compositions for staging and grading cancers. As already defined, staging relates to determining the extent of a cancer's spread, including its size and the degree to which other tissues, such as lymph nodes are involved in the cancer. Grading refers to the degree of a cell's retention of the characteristics of the tissue of its origin. A lower grade cancer comprises tumor cells that more closely resemble normal cells than a medium or higher grade cancer. Grading can be a useful diagnostic and prognostic tool. Higher grade cancers usually behave more aggressively than lower grade cancers. Thus, knowledge of the cancer grade, as well as its stage, can be a significant factor in the choice of the appropriate therapeutic intervention for the particular patient, e.g., surgery, radiation, chemotherapy, etc. Staging and grading can also be used in conjunction with a therapy to assess its efficacy, to determine prognosis, to determine effective dosages, etc.

Various methods of staging and grading cancers can be employed in accordance with the present invention. A “cell expression profile” or “cell expression fingerprint” is a representation of the expression of various different genes in a given cell or sample comprising cells. These cell expression profiles can be useful as reference standards. The cell expression fingerprints can be used alone for grading, or in combination with other grading methods.

The present invention also relates to methods and compositions for diagnosing a breast cancer, or determining susceptibility to a breast cancer, using polynucleotides, polypeptides, and specific-binding partners of the present invention to detect, assess, determine, etc., differentially-regulated genes of the present invention. In such methods, the gene can serve as a marker for breast cancer, e.g., where the gene, when mutant, is a direct cause of the breast cancer; where the gene is affected by another gene(s) which is directly responsible for the breast cancer, e.g., when the gene is part of the same signaling pathway as the directly responsible gene; and, where the gene is chromosomally linked to the gene(s) directly responsible for the breast cancer, and segregates with it. Many other situations are possible. To detect, assess, determine, etc., a probe specific for the gene can be employed as described above and below. Any method of detecting and/or assessing the gene can be used, including detecting expression of the gene using polynucleotides, antibodies, or other specific-binding partners.

The present invention relates to methods of diagnosing a disorder associated with breast cancer, or determining a subject's susceptibility to breast cancer, comprising, e.g., assessing the expression of a differentially-regulated gene in a tissue sample comprising tissue or cells suspected of having a breast cancer (e.g., where the sample comprises breast tissue). The phrase “diagnosing” indicates that it is determined whether the sample has a breast cancer. “Determining a subject's susceptibility to a breast cancer” indicates that the subject is assessed for whether s/he is predisposed to get such a disease or disorder, where the predisposition is indicated by abnormal expression of the gene (e.g., gene mutation, gene expression pattern is not normal, etc.). Predisposition or susceptibility to a disease may result when a such disease is influenced by epigenetic, environmental, etc., factors. This includes prenatal screening where samples from the fetus or embryo (e.g., via amniocentesis or CV sampling) are analyzed for the expression of the genes.

By the phrase “assessing expression of a differentially-regulated gene,” it is meant that the functional status of the gene is evaluated. This includes, but is not limited to, measuring expression levels of said gene, determining the genomic structure of said gene, determining the mRNA structure of transcripts from said gene, or measuring the expression levels of polypeptide coded for by said gene. Thus, the term “assessing expression” includes evaluating the all aspects of the transcriptional and translational machinery of the gene. For instance, if a promoter defect causes, or is suspected of causing, the disorder, then a sample can be evaluated (i.e., “assessed”) by looking (e.g., sequencing or restriction mapping) at the promoter sequence in the gene, by detecting transcription products (e.g., RNA), by detecting translation product (e.g., polypeptide). Any measure of whether the gene is functional can be used, including, polypeptide, polynucleotide, and functional assays for the gene's biological activity.

In making the assessment, it can be useful to compare the results to a normal gene, e.g., a gene which is not associated with the disorder. The nature of the comparison can be determined routinely, depending upon how the assessing is accomplished. If, for example, the mRNA levels of a sample is detected, then the mRNA levels of a normal can serve as a comparison, or a gene which is known not to be affected by the disorder. Methods of detecting mRNA are well known, and discussed above, e.g., but not limited to, Northern blot analysis, polymerase chain reaction (PCR), reverse transcriptase PCR, RACE PCR, etc.

Similarly, if polypeptide production is used to evaluate the gene, then the polypeptide in a normal tissue sample can be used as a comparison, or, polypeptide from a different gene whose expression is known not to be affected by the disorder. These are only examples of how such a method could be carried out.

Assessing the effects of therapeutic and preventative interventions (e.g., administration of a drug, chemotherapy, radiation, etc.) on breast cancer is a major effort in drug discovery, clinical medicine, and pharmacogenomics. The evaluation of therapeutic and preventative measures, whether experimental or already in clinical use, has broad applicability, e.g., in clinical trials, for monitoring the status of a patient, for analyzing and assessing animal models, and in any scenario involving cancer treatment and prevention. Analyzing the expression profiles of polynucleotides of the present invention can be utilized as a parameter by which interventions are judged and measured. Treatment of a disorder can change the expression profile in some manner which is prognostic or indicative of the drug's effect on it. Changes in the profile can indicate, e.g., drug toxicity, return to a normal level, etc. Accordingly, the present invention also relates to methods of monitoring or assessing a therapeutic or preventative measure (e.g., chemotherapy, radiation, anti-neoplastic drugs, antibodies, etc.) in a subject having breast cancer, or, susceptible to such a disorder, comprising, e.g., detecting the expression levels of one or more differentially-regulated genes of the present invention. A subject can be a cell-based assay system, non-human animal model, human patient, etc. Detecting can be accomplished as described for the methods above and below. By “therapeutic or preventative intervention,” it is meant, e.g., a drug administered to a patient, surgery, radiation, chemotherapy, and other measures taken to prevent, treat, or diagnose breast cancer.

Expression can be assessed in any sample comprising any tissue or cell type, body fluid, etc., as discussed for other methods of the present invention, including cells from breast cancer can be used, or cells derived from breast cancer. By the phrase “cells derived from breast cancer,” it is meant that the derived cells originate from breast cancer, e.g., when metastasis from a primary tumor site has occurred, when a progenitor-type or pluripotent cell gives rise to other cells, etc.

The present invention also relates to methods of using binding partners for differentially-regulated genes, such as antibodies, to deliver active agents to the breast for a variety of different purposes, including, e.g., for diagnostic, therapeutic (e.g., to treat cancer), and research purposes. Methods can involve delivering or administering an active agent to the breast cancer, comprising, e.g., administering to a subject in need thereof, an effective amount of an active agent coupled to a binding partner specific for a differentially-regulated gene polypeptide, wherein said binding partner is effective to deliver said active agent specifically to breast cancer.

Any type of active agent can be used in combination with the binding partner, including, therapeutic, cytotoxic, cytostatic, chemotherapeutic, anti-neoplastic, anti-proliferative, anti-biotic, etc., agents. A chemotherapeutic agent can be, e.g., DNA-interactive agent, alkylating agent, antimetabolite, tubulin-interactive agent, hormonal agent, hydroxyurea, Cisplatin, Cyclophosphamide, Altretamine, Bleomycin, Dactinomycin, Doxorubicin, Etoposide, Teniposide, paclitaxel, cytoxan, 2-methoxycarbonylaminobenzimidazole, Plicamycin, Methotrexate, Fluorouracil, Fluorodeoxyuridin, CB3717, Azacitidine, Floxuridine, Mercapyopurine, 6-Thioguanine, Pentostatin, Cytarabine, Fludarabine, etc. Agents can also be contrast agents useful in imaging technology, e.g., X-ray, CT, CAT, MRI, ultrasound, PET, SPECT, and scintographic.

An active agent can be associated in any manner with a binding partner which is effective to achieve its delivery specifically to the target. Specific delivery or targeting indicates that the agent is provided to the breast cancer, without being substantially provided to other tissues. This is useful especially where an agent is toxic, and specific targeting to the breast cancer enables the majority of the toxicity to be aimed at the breast cancer, with as small as possible effect on other tissues in the body. The association of the active agent and the binding partner (“coupling) can be direct, e.g., through chemical bonds between the binding partner and the agent, or, via a linking agent, or the association can be less direct, e.g., where the active agent is in a liposome, or other carrier, and the binding partner is associated with the liposome surface. In such case, the binding partner can be oriented in such a way that it is able to bind to the gene product on breast cell surface. Methods for delivery of DNA via a cell-surface receptor is described, e.g., in U.S. Pat. No. 6,339,139.

Identifying Agent Methods

The present invention also relates to methods of identifying agents, and the agents themselves, which modulate differentially regulated genes and gene products of the present invention. These agents can be used to modulate the biological activity of the polypeptide encoded for the gene, or the gene, itself. Agents which regulate the gene or its product are useful in variety of different environments, including as medicinal agents to treat or prevent disorders associated with differentially regulated genes and as research reagents to modify the function of tissues and cell.

Methods of identifying agents generally comprise steps in which an agent is placed in contact with the gene, transcription product, translation product, or other target, and then a determination is performed to assess whether the agent “modulates” the target. The specific method utilized will depend upon a number of factors, including, e.g., the target (i.e., is it the gene or polypeptide encoded by it), the environment (e.g., in vitro or in vivo), the composition of the agent, etc.

For modulating the expression of differentially-regulated genes of the present invention, a method can comprise, in any effective order, one or more of the following steps, e.g., contacting a differentially-regulated gene (e.g., in a cell population) with a test agent under conditions effective for said test agent to modulate the expression of said gene, and determining whether said test agent modulates said gene. An agent can modulate expression of a differentially-regulated gene at any level, including transcription, translation, and/or perdurance of the nucleic acid (e.g., degradation, stability, etc.) in the cell. For modulating the biological activity of polypeptides coded for by differentially-regulated genes, a method can comprise, in any effective order, one or more of the following steps, e.g., contacting a polypeptide (e.g., in a cell, lysate, or isolated) with a test agent under conditions effective for said test agent to modulate the biological activity of said polypeptide, and determining whether said test agent modulates said biological activity.

Contacting a differentially-regulated gene or polypeptide with the test agent can be accomplished by any suitable method and/or means that places the agent in a position to functionally control expression or biological activity of said gene or polypeptide present in the sample. Functional control indicates that the agent can exert its physiological effect on the gene or polypeptide through whatever mechanism it works. The choice of the method and/or means can depend upon the nature of the agent and the condition and type of environment in which the gene or polypeptide is presented, e.g., lysate, isolated, or in a cell population (such as, in vivo, in vitro, organ explants, etc.). For instance, if the cell population is an in vitro cell culture, the agent can be contacted with the cells by adding it directly into the culture medium. If the agent cannot dissolve readily in an aqueous medium, it can be incorporated into liposomes, or another lipophilic carrier, and then administered to the cell culture. Contact can also be facilitated by incorporation of agent with carriers and delivery molecules and complexes, by injection, by infusion, etc.

Agents can be directed to, or targeted to, any part of the polypeptide which is effective for modulating it. For example, agents, such as antibodies and small molecules, can be targeted to cell-surface, exposed, extracellular, ligand binding, functional, etc., domains of the polypeptide. Agents can also be directed to intracellular regions and domains, e.g., regions where the polypeptide couples or interacts with intracellular or intramembrane binding partners.

After the agent has been administered in such a way that it can gain access to the gene or polypeptide, it can be determined whether the test agent modulates their expression or biological activity. Modulation can be of any type, quality, or quantity, e.g., increase, facilitate, enhance, up-regulate, stimulate, activate, amplify, augment, induce, decrease, down-regulate, diminish, lessen, reduce, etc. The modulatory quantity can also encompass any value, e.g., 1%, 5%, 10%, 50%, 75%, 1-fold, 2-fold, 5-fold, 10-fold, 100-fold, etc. To modulate gene expression means, e.g., that the test agent has an effect on its expression, e.g., to effect the amount of transcription, to effect RNA splicing, to effect translation of the RNA into polypeptide, to effect RNA or polypeptide stability, to effect polyadenylation or other processing of the RNA, to effect post-transcriptional or post-translational processing, etc. To modulate biological activity means, e.g., that a functional activity of the polypeptide is changed in comparison to its normal activity in the absence of the agent. This effect includes, increase, decrease, block, inhibit, enhance, etc.

A test agent can be of any molecular composition, e.g., chemical compounds, biomolecules, such as polypeptides, lipids, nucleic acids (e.g., antisense to a polynucleotide sequence selected from SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147), carbohydrates, antibodies, ribozymes, double-stranded RNA, aptamers, etc. For example, if a polypeptide to be modulated is a cell-surface molecule, a test agent can be an antibody that specifically recognizes it and, e.g., causes the polypeptide to be internalized, leading to its down regulation on the surface of the cell. Such an effect does not have to be permanent, but can require the presence of the antibody to continue the down-regulatory effect. Antibodies can also be used to modulate the biological activity a polypeptide in a lysate or other cell-free form. Antisense can also be used as test agents to modulate gene expression.

Markers

The polynucleotides of the present invention can be used with other markers, especially breast and breast cancer markers to identity, detect, stage, diagnosis, determine, prognosticate, treat, etc., tissue, diseases and conditions, etc, of the breast. Markers can be polynucleotides, polypeptides, antibodies, ligands, specific binding partners, etc. The targets for such markers include, but are not limited genes and polypeptides that are selective for cell types present in the breast. Specific targets include, BRCA1, BRCA2, ATM, PTEN/MMAC1 (e.g., Ali et al., J. Natl. Cancer Inst., 91:1922-1932, 1999), MLH2, MSH2, TP53 (e.g., Done et al., Cancer Res., 58:785-789, 1998), STK 11, myc, cyclin D1 (e.g., Weinstat-Saslow et al., Nature Med., 1:1257-1260, 1995), c-erb-B2, keratins, such as 5/6 and 8/18.

Therapeutics

Selective polynucleotides, polypeptides, and specific-binding partners thereto, can be utilized in therapeutic applications, especially to treat breast cancer. Useful methods include, but are not limited to, immunotherapy (e.g., using specific-binding partners to polypeptides), vaccination (e.g., using a selective polypeptide or a naked DNA encoding such polypeptide), protein or polypeptide replacement therapy, gene therapy (e.g., germ-line correction, antisense), etc.

Various immunotherapeutic approaches can be used. For instance, unlabeled antibody that specifically recognizes a tissue-specific antigen can be used to stimulate the body to destroy or attack the cancer, to cause down-regulation, to produce complement-mediated lysis, to inhibit cell growth, etc., of target cells which display the antigen, e.g., analogously to how c-erbB-2 antibodies are used to treat breast cancer. In addition, antibody can be labeled or conjugated to enhance its deleterious effect, e.g., with radionuclides and other energy emitting entitities, toxins, such as ricin, exotoxin A (ETA), and diphtheria, cytotoxic or cytostatic agents, immunomodulators, chemotherapeutic agents, etc. See, e.g., U.S. Pat. No. 6,107,090.

An antibody or other specific-binding partner can be conjugated to a second molecule, such as a cytotoxic agent, and used for targeting the second molecule to a tissue-antigen positive cell (Vitetta, E. S. et al., 1993, Immunotoxin therapy, in DeVita, Jr., V. T. et al., eds, Cancer: Principles and Practice of Oncology, 4th ed., J. B. Lippincott Co., Philadelphia, 2624-2636). Examples of cytotoxic agents include, but are not limited to, antimetabolites, alkylating agents, anthracyclines, antibiotics, anti-mitotic agents, radioisotopes and chemotherapeutic agents. Further examples of cytotoxic agents include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, 1-dehydrotestosterone, diptheria toxin, Pseudomonas exotoxin (PE) A, PE40, abrin, elongation factor-2 and glucocorticoid. Techniques for conjugating therapeutic agents to antibodies are well.

In addition to immunotherapy, polynucleotides and polypeptides can be used as targets for non-immunotherapeutic applications, e.g., using compounds which interfere with function, expression (e.g., antisense as a therapeutic agent), assembly, etc. RNA interference can be used in vitro and in vivo to silence differentially-expressed genes when its expression contributes to a disease (but also for other purposes, e.g., to identify the gene's function to change a developmental pathway of a cell, etc.). See, e.g., Sharp and Zamore, Science, 287:2431-2433, 2001; Grishok et al., Science, 287:2494, 2001.

Delivery of therapeutic agents can be achieved according to any effective method, including, liposomes, viruses, plasmid vectors, bacterial delivery systems, orally, systemically, etc. Therapeutic agents of the present invention can be administered in any form by any effective route, including, e.g., oral, parenteral, enteral, intraperitoneal, topical, transdermal (e.g., using any standard patch), ophthalmic, nasally, local, non-oral, such as aerosal, inhalation, subcutaneous, intramuscular, buccal, sublingual, rectal, vaginal, intra-arterial, and intrathecal, etc. They can be administered alone, or in combination with any ingredient(s), active or inactive.

In addition to therapeutics, per se, the present invention also relates to methods of treating breast cancer showing altered expression of differentially-regulated genes, such as SEQ ID NOS 1-92 and 116-147, comprising, e.g., administering to a subject in need thereof a therapeutic agent which is effective for regulating expression of said genes and/or which is effective in treating said disease. The term “treating” is used conventionally, e.g., the management or care of a subject for the purpose of combating, alleviating, reducing, relieving, improving the condition of, etc., of a disease or disorder. By the phrase “altered expression,” it is meant that the disease is associated with a mutation in the gene, or any modification to the gene (or corresponding product) which affects its normal function. Thus, expression of a differentially-regulated gene refers to, e.g., transcription, translation, splicing, stability of the mRNA or protein product, activity of the gene product, differential expression, etc.

Any agent which “treats” the disease can be used. Such an agent can be one which regulates the expression of the gene. Expression refers to the same acts already mentioned, e.g. transcription, translation, splicing, stability of the mRNA or protein product, activity of the gene product, differential expression, etc. For instance, if the condition was a result of a complete deficiency of the gene product, administration of gene product to a patient would be said to treat the disease and regulate the gene's expression. Many other possible situations are possible, e.g., where the gene is aberrantly expressed, and the therapeutic agent regulates the aberrant expression by restoring its normal expression pattern.

Antisense

Antisense polynucleotide (e.g., RNA) can also be prepared from a polynucleotide according to the present invention, preferably an anti-sense to a sequence of SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147. Antisense polynucleotide can be used in various ways, such as to regulate or modulate expression of the polypeptides they encode, e.g., inhibit their expression, for in situ hybridization, for therapeutic purposes, for making targeted mutations (in vivo, triplex, etc.) etc. For guidance on administering and designing anti-sense, see, e.g., U.S. Pat. Nos. 6,200,960, 6,200,807, 6,197,584, 6,190,869, 6,190,661, 6,187,587, 6,168,950, 6,153,595, 6,150,162, 6,133,246, 6,117,847, 6,096,722, 6,087,343, 6,040,296, 6,005,095, 5,998,383, 5,994,230, 5,891,725, 5,885,970, and 5,840,708. An antisense polynucleotides can be operably linked to an expression control sequence. A total length of about 35 bp can be used in cell culture with cationic liposomes to facilitate cellular uptake, but for in vivo use, preferably shorter oligonucleotides are administered, e.g. 25 nucleotides.

Antisense polynucleotides can comprise modified, nonnaturally-occurring nucleotides and linkages between the nucleotides (e.g., modification of the phosphate-sugar backbone; methyl phosphonate, phosphorothioate, or phosphorodithioate linkages; and 2′-O-methyl ribose sugar units), e.g., to enhance in vivo or in vitro stability, to confer nuclease resistance, to modulate uptake, to modulate cellular distribution and compartmentalization, etc. Any effective nucleotide or modification can be used, including those already mentioned, as known in the art, etc., e.g., disclosed in U.S. Pat. Nos. 6,133,438; 6,127,533; 6,124,445; 6,121,437; 5,218,103 (e.g., nucleoside thiophosphoramidites); 4,973,679; Sproat et al., “2′-O-Methyloligoribonucleotides: synthesis and applications,” Oligonucleotides and Analogs A Practical Approach, Eckstein (ed.), IRL Press, Oxford, 1991, 49-86; Iribarren et al., “2′-O-Alkyl Oligoribonucleotides as Antisense Probes,” Proc. Natl. Acad. Sci. USA, 1990, 87, 7747-7751; Cotton et al., “2′-O-methyl, 2′-O-ethyl oligoribonucleotides and phosphorothioate oligodeoxyribonucleotides as inhibitors of the in vitro U7 snRNP-dependent mRNA processing event,” Nucl. Acids Res., 1991, 19, 2629-2635.

Arrays

The present invention also relates to an ordered array of polynucleotide probes and specific-binding partners (e.g., antibodies) for detecting the expression of differentially-regulated genes in a sample, comprising, one or more polynucleotide probes or specific binding partners associated with a solid support, wherein each probe is specific for said genes, and the probes comprise a nucleotide sequence of SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147, which is specific for said gene, a nucleotide sequence having sequence identity to SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, and/or 147, which is specific for said gene or polynucleotide, or complements thereto, or a specific-binding partner which is specific for said genes.

The phrase “ordered array” indicates that the probes are arranged in an identifiable or position-addressable pattern, e.g., such as the arrays disclosed in U.S. Pat. Nos. 6,156,501, 6,077,673, 6,054,270, 5,723,320, 5,700,637, WO09919711, WO00023803. The probes are associated with the solid support in any effective way. For instance, the probes can be bound to the solid support, either by polymerizing the probes on the substrate, or by attaching a probe to the substrate. Association can be, covalent, electrostatic, noncovalent, hydrophobic, hydrophilic, noncovalent, coordination, adsorbed, absorbed, polar, etc. When fibers or hollow filaments are utilized for the array, the probes can fill the hollow orifice, be absorbed into the solid filament, be attached to the surface of the orifice, etc. Probes can be of any effective size, sequence identity, composition, etc., as already discussed.

Ordered arrays can further comprise polynucleotide probes or specific-binding partners which are specific for other genes, including genes specific for breast cancer or disorders associated with breast tissue.

Transgenic Animals

The present invention also relates to transgenic animals comprising differentially-regulated genes (and corresponding homologs thereof) of the present invention. Such genes, as discussed in more detail below, include, but are not limited to, functionally-disrupted genes, mutated genes, ectopically or selectively-expressed genes, inducible or regulatable genes, etc. These transgenic animals can be produced according to any suitable technique or method, including homologous recombination, mutagenesis (e.g., ENU, Rathkolb et al., Exp. Physiol., 85(6):635-644, 2000), and the tetracycline-regulated gene expression system (e.g., U.S. Pat. No. 6,242,667). The term “gene” as used herein includes any part of a gene, i.e., regulatory sequences, promoters, enhancers, exons, introns, coding sequences, etc. The nucleic acid present in the construct or transgene can be naturally-occurring wild-type, polymorphic, or mutated. When a mouse or other mammal is used, the appropriate homolog can be used in place of a human gene of the present invention.

Along these lines, polynucleotides of the present invention can be used to create transgenic animals, e.g. a non-human animal, comprising at least one cell whose genome comprises a functional disruption of a differentially-regulated gene of the present invention (including homologs of them). By the phrases “functional disruption” or “functionally disrupted,” it is meant that the gene does not express a biologically-active product. It can be substantially deficient in at least one functional activity coded for by the gene. Expression of a polypeptide can be substantially absent, i.e., essentially undetectable amounts are made. However, polypeptide can also be made, but which is deficient in activity, e.g., where only an amino-terminal portion of the gene product is produced.

Functional disruptions can be made to the regions of the gene which are specific or unique to that gene, e.g., as disclosed herein. For instance, BCU0021 has a unique N-terminal region (e.g., amino acids 1-105 not shared by publicly available genes). A functional disruption can be made in this portion of the gene, or upstream to it. The transgenic animal can comprise one or more cells. When substantially all its cells contain the engineered gene, it can be referred to as a transgenic animal “whose genome comprises” the engineered gene. This indicates that the endogenous gene loci of the animal has been modified and substantially all cells contain such modification.

Functional disruption of the gene can be accomplished in any effective way, including, e.g., introduction of a stop codon into any part of the coding sequence such that the resulting polypeptide is biologically inactive (e.g., because it lacks a catalytic domain, a ligand binding domain, etc.), introduction of a mutation into a promoter or other regulatory sequence that is effective to turn it off, or reduce transcription of the gene, insertion of an exogenous sequence into the gene which inactivates it (e.g., which disrupts the production of a biologically-active polypeptide or which disrupts the promoter or other transcriptional machinery), deletion of sequences from the a differentially-regulated gene, etc. Examples of transgenic animals having functionally disrupted genes are well known, e.g., as described in U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. A transgenic animal which comprises the functional disruption can also be referred to as a “knock-out” animal, since the biological activity of its a differentially-regulated gene has been “knocked-out.” Knock-outs can be homozygous or heterozygous.

For creating functional disrupted genes, and other gene mutations, homologous recombination technology is of special interest since it allows specific regions of the genome to be targeted. Using homologous recombination methods, genes can be specifically-inactivated, specific mutations can be introduced, and exogenous sequences can be introduced at specific sites. These methods are well known in the art, e.g., as described in the patents above. See, also, Robertson, Biol. Reproduc., 44(2):238-245, 1991. Generally, the genetic engineering is performed in an embryonic stem (ES) cell, or other pluripotent cell line (e.g., adult stem cells, EG cells), and that genetically-modified cell (or nucleus) is used to create a whole organism. Nuclear transfer can be used in combination with homologous recombination technologies.

For example, a differentially-regulated gene locus can be disrupted in mouse ES cells using a positive-negative selection method (e.g., Mansour et al., Nature, 336:348-352, 1988). In this method, a targeting vector can be constructed which comprises a part of the gene to be targeted. A selectable marker, such as neomycin resistance genes, can be inserted into a a differentially-regulated gene exon present in the targeting vector, disrupting it. When the vector recombines with the ES cell genome, it disrupts the function of the gene. The presence in the cell of the vector can be determined by expression of neomycin resistance. See, e.g., U.S. Pat. No. 6,239,326. Cells having at least one functionally disrupted gene can be used to make chimeric and germline animals, e.g., animals having somatic and/or germ cells comprising the engineered gene. Homozygous knock-out animals can be obtained from breeding heterozygous knock-out animals. See, e.g., U.S. Pat. No. 6,225,525.

A transgenic animal, or animal cell, lacking one or more functional differentially-regulated genes can be useful in a variety of applications, including, as an animal model for cancer, for drug screening assays, as a source of tissues deficient in said gene activity, and any of the utilities mentioned in any issued U.S. Patent on transgenic animals, including, U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824.

The present invention also relates to non-human, transgenic animal whose genome comprises recombinant a differentially-regulated gene nucleic acid operatively linked to an expression control sequence effective to express said coding sequence, e.g., in breast cancer such a transgenic animal can also be referred to as a “knock-in” animal since an exogenous gene has been introduced, stably, into its genome.

A recombinant a differentially-regulated gene nucleic acid refers to a gene which has been introduced into a target host cell and optionally modified, such as cells derived from animals, plants, bacteria, yeast, etc. A recombinant a differentially-regulated gene includes completely synthetic nucleic acid sequences, semi-synthetic nucleic acid sequences, sequences derived from natural sources, and chimeras thereof. “Operable linkage” has the meaning used through the specification, i.e., placed in a functional relationship with another nucleic acid. When a gene is operably linked to an expression control sequence, as explained above, it indicates that the gene (e.g., coding sequence) is joined to the expression control sequence (e.g., promoter) in such a way that facilitates transcription and translation! of the coding sequence. As described above, the phrase “genome” indicates that the genome of the cell has been modified. In this case, the recombinant a differentially-regulated gene has been stably integrated into the genome of the animal. The a differentially-regulated gene nucleic acid in operable linkage with the expression control sequence can also be referred to as a construct or transgene.

Any expression control sequence can be used depending on the purpose. For instance, if selective expression is desired, then expression control sequences which limit its expression can be selected. These include, e.g., tissue or cell-specific promoters, introns, enhancers, etc. For various methods of cell and tissue-specific expression, see, e.g., U.S. Pat. Nos. 6,215,040, 6,210,736, and 6,153,427. These also include the endogenous promoter, i.e., the coding sequence can be operably linked to its own promoter. Inducible and regulatable promoters can also be utilized.

The present invention also relates to a transgenic animal which contains a functionally disrupted and a transgene stably integrated into the animals genome. Such an animal can be constructed using combinations any of the above- and below-mentioned methods. Such animals have any of the aforementioned uses, including permitting the knock-out of the normal gene and its replacement with a mutated gene. Such a transgene can be integrated at the endogenous gene locus so that the functional disruption and “knock-in” are carried out in the same step.

In addition to the methods mentioned above, transgenic animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology, cloning methods, nuclear transfer methods. See, also, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-7384, 1980; Palmiter et al., Cell, 41:343-345, 1985; Palmiter et al., Ann. Rev. Genet., 20:465499, 1986; Askew et al., Mol. Cell. Bio., 13:4115-4124, 1993; Games et al. Nature, 373:523-527, 1995; Valancius and Smithies, Mol. Cell. Bio., 11: 1402-1408, 1991; Stacey et al., Mol. Cell. Bio., 14:1009-1016, 1994; Hasty et al., Nature, 350:243-246, 1995; Rubinstein et al., Nucl. Acid Res., 21:2613-2617,1993; Cibelli et al., Science, 280:1256-1258, 1998. For guidance on recombinase excision systems, see, e.g., U.S. Pat. Nos. 5,626,159, 5,527,695, and 5,434,066. See also, Orban, P. C., et al., “Tissue- and Site-Specific DNA Recombination in Transgenic Mice,” Proc. Natl. Acad. Sci. USA, 89:6861-6865 (1992); O'Gorman, S., et al., “Recombinase-Mediated Gene Activation and Site-Specific Integration in Mammalian Cells,” Science, 251:1351-1355 (1991); Sauer, B., et al., “Cre-stimulated recombination at loxP-Containing DNA sequences placed into the mammalian genome,” Polynucleotides Research, 17(1): 147-161 (1989); Gagneten, S. et al. (1997) Nucl. Acids Res. 25:3326-3331; Xiao and Weaver (1997) Nucl. Acids Res. 25:2985-2991; Agah, R. et al. (1997) J. Clin. Invest. 100: 169-179; Barlow, C. et al. (1997) Nucl. Acids Res. 25:2543-2545; Araki, K. et al. (1997) Nucl. Acids Res. 25:868-872; Mortensen, R. N. et al. (1992) Mol. Cell. Biol. 12:2391-2395 (G418 escalation method); Lakhlani, P. P. et al. (1997) Proc. Natl. Acad. Sci. USA 94:9950-9955 (“hit and run”); Westphal and Leder (1997) Curr. Biol. 7:530-533 (transposon-generated “knock-out” and “knock-in”); Templeton, N. S. et al. (1997) Gene Ther. 4:700-709 (methods for efficient gene targeting, allowing for a high frequency of homologous recombination events, e.g., without selectable markers); PCT International Publication WO 93/22443 (functionally-disrupted).

A polynucleotide according to the present invention can be introduced into any non-human animal, including a non-human mammal, mouse (Hogan et al., Manipulating the Mouse Embryo: A Laboratory Manual, Cold Spring Harbor Laboratory, Cold Spring Harbor, N.Y., 1986), pig (Hammer et al., Nature, 315:343-345, 1985), sheep (Hammer et al., Nature, 315:343-345, 1985), cattle, rat, or primate. See also, e.g., Church, 1987, Trends in Biotech 5:13-19; Clark et al., Trends in Biotech. 5:20-24, 1987); and DePamphilis et al., BioTechniques, 6:662-680, 1988. Transgenic animals can be produced by the methods described in U.S. Pat. No. 5,994,618, and utilized for any of the utilities described therein.

Database

The present invention also relates to electronic forms of polynucleotides, polypeptides, etc., of the present invention, including computer-readable medium (e.g., magnetic, optical, etc., stored in any suitable format, such as flat files or hierarchical files) which comprise such sequences, or fragments thereof, e-commerce-related means, etc. Along these lines, the present invention relates to methods of retrieving gene sequences from a computer-readable medium, comprising, one or more of the following steps in any effective order, e.g., selecting a cell or gene expression profile, e.g., a profile that specifies that said gene is differentially expressed in breast cancer, and retrieving said differentially expressed gene sequences, where the gene sequences consist of the genes represented by SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, 147, or homologs and variants thereof.

A “gene expression profile” means the list of tissues, cells, etc., in which a defined gene is expressed (i.e, transcribed and/or translated). A “cell expression profile” means the genes which are expressed in the particular cell type. The profile can be a list of the tissues in which the gene is expressed, but can include additional information as well, including level of expression (e.g., a quantity as compared or normalized to a control gene), and information on temporal (e.g., at what point in the cell-cycle or developmental program) and spatial expression. By the phrase “selecting a gene or cell expression profile,” it is meant that a user decides what type of gene or cell expression pattern he is interested in retrieving, e.g., he may require that the gene is differentially expressed in a tissue, or he may require that the gene is not expressed in blood, but must be expressed in breast tissue. Any pattern of expression preferences may be selected. The selecting can be performed by any effective method. In general, “selecting” refers to the process in which a user forms a query that is used to search a database of gene expression profiles. The step of retrieving involves searching for results in a database that correspond to the query set forth in the selecting step. Any suitable algorithm can be utilized to perform the search query, including algorithms that look for matches, or that perform optimization between query and data. The database is information that has been stored in an appropriate storage medium, having a suitable computer-readable format. Once results are retrieved, they can be displayed in any suitable format, such as HTML.

For instance, the user may be interested in identifying genes that are differentially expressed in a breast cancer. He may not care whether small amounts of expression occur in other tissues, as long as such genes are not expressed in peripheral blood lymphocytes (see above for examples). A query is formed by the user to retrieve the set of genes from the database having the desired gene or cell expression profile. Once the query is inputted into the system, a search algorithm is used to interrogate the database, and retrieve results.

Advertising, Licensing, Etc., Methods

The present invention also relates to methods of advertising, licensing, selling, purchasing, brokering, etc., genes, polynucleotides, specific-binding partners, antibodies, etc., of the present invention. Methods can comprises, e.g., displaying a a differentially-regulated gene gene, a differentially-regulated gene polypeptide, or antibody specific for a differentially-regulated gene in a printed or computer-readable medium (e.g., on the Web or Internet), accepting an offer to purchase said gene, polypeptide, or antibody.

Other

A polynucleotide, probe, polypeptide, antibody, specific-binding partner, etc., according to the present invention can be isolated. The term “isolated” means that the material is in a form in which it is not found in its original environment or in nature, e.g., more concentrated, more purified, separated from component, etc. An isolated polynucleotide includes, e.g., a polynucleotide having the sequenced separated from the chromosomal DNA found in a living animal, e.g., as the complete gene, a transcript, or a cDNA. This polynucleotide can be part of a vector or inserted into a chromosome (by specific gene-targeting or by random integration at a position other than its normal position) and still be isolated in that it is not in a form that is found in its natural environment. A polynucleotide, polypeptide, etc., of the present invention can also be substantially purified. By substantially purified, it is meant that polynucleotide or polypeptide is separated and is essentially free from other polynucleotides or polypeptides, i.e., the polynucleotide or polypeptide is the primary and active constituent. A polynucleotide can also be a recombinant molecule. By “recombinant,” it is meant that the polynucleotide is an arrangement or form which does not occur in nature. For instance, a recombinant molecule comprising a promoter sequence would not encompass the naturally-occurring gene, but would include the promoter operably linked to a coding sequence not associated with it in nature, e.g., a reporter gene, or a truncation of the normal coding sequence.

The term “marker” is used herein to indicate a means for detecting or labeling a target. A marker can be a polynucleotide (usually referred to as a “probe”), polypeptide (e.g., an antibody conjugated to a detectable label), PNA, or any effective material.

The topic headings set forth above are meant as guidance where certain information can be found in the application, but are not intended to be the only source in the application where information on such topic can be found.

Reference Materials

For other aspects of the polynucleotides, reference is made to standard textbooks of molecular biology. See, e.g., Hames et al., Polynucleotide Hybridization, IL Press, 1985; Davis et al., Basic Methods in Molecular Biology, Elsevir Sciences Publishing, Inc., New York, 1986; Sambrook et al., Molecular Cloning, CSH Press, 1989; Howe, Gene Cloning and Manipulation, Cambridge University Press, 1995; Ausubel et al., Current Protocols in Molecular Biology, John Wiley & Sons, Inc., 1994-1998.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever. The entire disclosure of all applications, patents and publications, cited above and in the figures are hereby incorporated by reference in their entirety.

TABLE 1

| Clone ID | Protein-L* | Location | Domains Names |
|---|---|---|---|
| 1. Bcd0468a | 98aa | Membrane | Signal peptide: 1-28aa |
| | | | Transmembrane domain: 20-42aa. (Partial CDS) |
| 1. Bcd0468b | 112aa | Membrane | Signal peptide: 1-29aa; (Partial CDS) |
| | | | Transmembrane: 20-42aa. |
| 1. Bcd0468c | 110aa | n/a | No domain found in the current protein databases. |
| 2. Bcu0021z | 404aa | Intracellular | Nmra domain: 118-320aa. |
| 3. Bcu0067z | 228aa | Membrane | Transmembrane: 13-35aa. |
| 4. Bcu0120 | 57aa | n/a | No domain found in the current protein databases. |
| 5. Bcu0148 | 97aa | n/a | No domain found in the current protein databases. |
| 6. Bcu0149 | 124aa | Membrane | 1. Transmembrane 7-26aa; |
| | | | 2. BCL domain: 32-64aa; |
| | | | 3. TOP2c domain: 74-113aa. |
| 7. Bcu0092 | 234aa | Membrane | 1. Transmembrane domain: 55-74aa; |
| | | | 2. Transmembrane domain: 94-116aa; |
| | | | 3. Transmembrane domain: 123-142aa; |
| | | | 4. Transmembrane domain: 152-171aa. |
| 8. Bcu0156x | 2677aa | Nuclear | 1. DEXDc3 domain: 1930-2244aa |
| | | | 2. Urdu/REP helicase domain: 1935-2210aa; |
| | | | 3. ATP/GTP-binding site motif A (P-loop): 1963-1970aa; |
| | | | 4. Nuclear localization signal: 2070-2087aa. |
| 9. Bcu0258x | 1078aa | Membrane- | 1. Myosin_head (large ATPase) domain: 17-688aa; |
| | | associated1 | 2. IQ motif: 703-725aa; |
| | | | 3. IQ motif: 726-748aa; |
| | | | 4. IQ motif: 749-771aa; |
| | | | 5. IQ motif: 778-800aa. |
| 10. Bcu0343 | 811aa | Nuclear | 1. KRAB domain: 12-72aa; |
| | | | 2. Internal repeat domain: 140-279aa; |
| | | | 3. ZnF_C2H2 domain: 328-350aa; |
| | | | 4. ZnF_C2H2 domain: 356-378aa; |
| | | | 5. ZnF_C2H2 domain: 384-406aa; |
| | | | 6. ZnF_C2H2 domain: 412-434aa; |
| | | | 7. ZnF_C2H2 domain: 440-462aa; |
| | | | 8. ZnF_C2H2 domain: 468-490aa; |
| | | | 9. ZnF_C2H2 domain: 496-518aa; |
| | | | 10. ZnF_C2H2 domain: 524-54aa; |
| | | | 11. ZnF_C2H2 domain: 552-574aa; |
| | | | 12. ZnF_C2H2 domain: 580-602aa; |
| | | | 13. ZnF_C2H2 domain: 608-630aa; |
| | | | 14. ZnF_C2H2 domain: 636-658aa; |
| | | | 15. ZnF_C2H2 domain: 664-686aa; |
| | | | 16. ZnF_C2H2 domain: 692-714aa; |
| | | | 17. ZnF_C2H2 domain: 720-742aa; |
| | | | 18. ZnF_C2H2 domain: 748-770aa; |
| | | | 19. LIM domain: 637-697aa. |
| | | | 20. ZnF_GATA domain: 688-746aa. |
| | | | 21. ZnF_BED domain: 730-773aa. |
| 11a. Bcu371Az | 199aa | Nuclear | No domain found in the current protein databases. |
| 11b. Bcu0371Bz | 237aa | Nuclear | Basic region leucine zipper (BRLZ) domain: 153-217aa |
| 12. Bcu399 | 487aa | Membrane | 1. Transmembrane domain: 49-71aa; |
| | | | 2. Transmembrane domain: 86-103aa. |
| 13. Bcu0408z | 504aa | Nuclear | 1. Coiled coil domain: 51-81aa. |
| | | | 2. Protein kinase (unclassified specificity) domain: |
| | | | 228-331aa. |
| 14. Bcu0475z | 191aa | Cytoplasm | 1. Thioredoxin-like domain: 95-176aa. |
| 15. Bcu0504 | 367aa | Cytoplasm | 1. WD40 domains: 50-89aa; |
| | | | 2. WD40 domains: 92-131aa; |
| | | | 3. WD40 domains: 135-174aa; |
| | | | 4. WD40 domains: 177-216aa; |
| | | | 5. WD40 domains: 267-310aa; |
| | | | 6. WD40 domains: 313-352aa. |
| 16. Bcu0571 | 128aa | Cytoplasm | No domain found in the current protein databases. |
| 17. Bcu0720x | 1337aa | Nuclear | No domain found in the current protein databases. |
| 18. Bcu0721z | 1179aa | Membrane | 1. Cysteine-rich repeat in FGFR: 153-212aa; |
| | | | 2. Cysteine-rich repeat in FGFR: 291-345aa; |
| | | | 3. Cysteine-rich repeat in FGFR: 349-413aa |
| | | | 4. Cysteine-rich repeat in FGFR: 417-473aa; |
| | | | 5. Cysteine-rich repeat in FGFR: 480-537aa; |
| | | | 6. Cysteine-rich repeat in FGFR: 540-604aa; |
| | | | 7. Cysteine-rich repeat in FGFR: 612-666aa; |
| | | | 8. Cysteine-rich repeat in FGFR: 670-728aa; |
| | | | 9. Cysteine-rich repeat in FGFR: 732-788aa; |
| | | | 10. Cysteine-rich repeat in FGFR: 800-854aa; |
| | | | 11. Cysteine-rich repeat in FGFR: 858-911aa; |
| | | | 12. Cysteine-rich repeat in FGFR: 915-972aa; |
| | | | 13. Cysteine-rich repeat in FGFR: 982-1042aa; |
| | | | 14. Cysteine-rich repeat in FGFR: 1046-1011aa; |
| | | | 15. Transmembrane domain: 1146-1168aa. |
| 19. Bcu0730Ax | 543aa | Cytoplasm | No domain found in the current protein databases. |
| 19. Bcu0730Bx | 516aa | Cytoplasm | No domain found in the current protein databases. |
| 19. Bcu0730Cx | 538aa | Cytoplasm | No domain found in the current protein databases. |
| 19. Bcu0730Dx | 585aa | Cytoplasm | No domain found in the current protein databases. |
| 20. Bcu770 | 463aa | Extracellular | 1. FN1 domain-1: 88-123aa; |
| | | | 2. FN1 domain-2: 133-171aa; |
| | | | 3. FN1 domain-3: 177-215aa; |
| | | | 4. FN1 domain-4: 222-261aa; |
| | | | 5. FN1 domain-5: 267-306aa; |
| | | | 6. FN1 domain-6: 344-378aa; |
| | | | 7. FN2 domain-1: 393-403aa; |
| | | | 8. FN2 domain-2: 404-417aa; |
| | | | 9. FN2 domain-3: 421-437aa; |
| | | | 10. Glycosyl hydrolase family domain-1: 118-145aa; |
| | | | 11. Glycosyl hydrolase family domain-2: 365-393aa; |
| | | | 12. EGF-like domain: 112-123aa. |
| 21. Bcu0840 | 723aa | Nuclear | TBOX domain: 102-290aa. |
| 22. Bcu0862 | 112aa | Nuclear | No domain found in the current protein databases. |
| 23. Bcu0916z | 118aa | Nuclear | No domain found in the current protein databases. |
| 24. Bcu0918-2 | 813aa | Cytoplasm | No domain found in the current protein databases. |
| 25. Bcu0947xz | 3051aa | Nuclear | 1. Proline-rich region: 5-36aa; |
| | | | 2. Coiled coil: 288-310aa; |
| | | | 3. Coiled coil: 350-374aa; |
| | | | 4. Coiled coil: 497-527aa; |
| | | | 5. Coiled coil: 554-599aa; |
| | | | 6. Coiled coil: 818-841aa; |
| | | | 7. CT domain: 1309-2187aa; |
| | | | 8. AT_hook domain: 2872-2884aa; |
| | | | 9. TT_ORF2 domain: 77-129aa. |
| 26. Bcu1034 | 264aa | Extracellular | 1. Signal peptide: 1-21aa |
| | | | 2. Thrombospondin type 1 repeats (TSP1) domain: |
| | | | 77-133aa. |
| 27. Bcu1041 | 614aa | Nuclear | 1. bZIP domain: 228-275aa. |
| 28. Bcu0610Az | 582aa | Nuclear | 1. RNA recognition motif: 126-188aa; |
| | | | 2. Lupus La protein domain: 41-59aa; |
| | | | 3. Lupus La protein domain: 67-83aa; |
| | | | 4. Lupus La protein domain: 98-112aa; |
| | | | 5. Lupus La protein domain: 165aa-184aa. |
| 28. Bcu0610Bz | 582aa | Same as Bcu0610Az. | |
| 29. Bcu0586 | 2376aa | Cytoplasm | Not found in current protein databases. |
| 30. Bcu0715Az | 791aa | Cytoplasm | 1. Calpain inhibitor domain: 177-307aa; |
| | | | 2. Calpain inhibitor domain: 312-442aa; |
| | | | 3. Calpain inhibitor domain: 447-584aa; |
| | | | 4. Calpain inhibitor domain: 590-720aa. |
| 30. Bcu0715Bz | 769aa | Cytoplasm | 1. Calpain inhibitor domain: 155-285aa; |
| | | | 2. Calpain inhibitor domain: 290-420aa; |
| | | | 3. Calpain inhibitor domain: 425-562aa; |
| | | | 4. Calpain inhibitor domain: 568-698aa. |
| 30. Bcu0715Cz | 750aa | Cytoplasm | 1. Calpain inhibitor domain: 136-266aa; |
| | | | 2. Calpain inhibitor domain: 271-401aa; |
| | | | 3. Calpain inhibitor domain: 406-543aa; |
| | | | 4. Calpain inhibitor domain: 549-679aa. |
| 31. Bcu0205Az | 2551aa | Membrane | 1. Transmembrane domain: 233-245aa; |
| | | | 2. Epidermal growth factor-like domain: 423-451aa; |
| | | | 3. Epidermal growth factor-like domain: 454-482aa; |
| | | | 4. Epidermal growth factor-like domain: 487-516aa; |
| | | | 5. Epidermal growth factor-like domain: 553-583aa; |
| | | | 6. Calcium-binding EGF-like domain: 517-548aa; |
| | | | 7. Calcium-binding EGF-like domain: 583-618aa. |
| 31. Bcu0205Bz | 2633aa | Membrane | 1. Transmembrane domain: 305-327aa; |
| | | | 2. Epidermal growth factor-like domain: 505-533aa; |
| | | | 3. Epidermal growth factor-like domain: 536-564aa; |
| | | | 4. Epidermal growth factor-like domain: 569-598aa; |
| | | | 5. Epidermal growth factor-like domain: 635-665aa; |
| | | | 6. Calcium-binding EGF-like domain: 601-630aa; |
| | | | 7. Calcium-binding EGF-like domain: 668-700aa. |
| 32. Bcu0988Az | 843aa | Nuclear | 1. RRM domain: 88-160aa (RNA recognition |
| | | | motif); |
| | | | 2. Coiled coil: 271-352aa; |
| | | | 3. Coiled coil: 355-554aa; |
| | | | 3. PWI domain (In splicing factors): 763-836aa. |
| 32. Bcu0988Bz | 779aa | Nuclear | 1. RRM domain: 88-160aa (RNA recognition motif) |
| | | | 2. Coiled coil: 271-490aa; |
| | | | 3. PWI domain (In splicing factors): 699-772aa. |
| 33. Bcu0518z | 1906aa | Nuclear | 1. S1(ribosomal protein S1-like RNA-binding) |
| | | | domain: 116-206aa; |
| | | | 2. S1 domain: 220-293aa; |
| | | | 3. S1 domain: 314-381aa; |
| | | | 4. S1 domain: 398-471aa; |
| | | | 5. S1 domain: 486-557aa; |
| | | | 6. S1 domain: 575-646aa; |
| | | | 7. S1 domain: 669-742aa; |
| | | | 8. S1 domain: 762-833aa; |
| | | | 9. S1 domain: 879-946aa; |
| | | | 10. S1 domain: 1069-1144aa; |
| | | | 11. S1 domain: 1182-1257aa; |
| | | | 12. S1 domain: 1263-1333aa; |
| | | | 13. S1 domain: 1357-1431aa; |
| | | | 14. Coiled coil domain: 1441-1480aa; |
| | | | 15. Coiled coil domain: 1601-1632aa; |
| | | | 16. HAT domain: 1635-1666aa; |
| | | | 17. HAT domain: 1668-1705aa; |
| | | | 18. HAT domain: 1707-1738aa; |
| | | | 19. HAT domain: 1740-1772aa; |
| | | | 20. HAT domain: 1774-1808aa; |
| | | | 20. HAT domain: 1774-1808aa; |
| | | | 21. HAT domain: 1810-1842aa; |
| | | | 22. HAT domain: 1844-1879aa; |
| | | | 23. TRP repeat domain: 1726-1829aa. |
| 34. Bcu0147Az | 958aa | Nuclear | 1. R3H domain: 152-229aa; |
| | | | (Single-strand nucleic acids-binding) |
| | | | 2. Proline-rich region: 606-704aa. |
| 34. Bcu0147Bz | 976aa | Nuclear | 1. R3H domain: 152-229aa; |
| | | | (Single-strand nucleic acids-binding) |
| | | | 2. Proline-rich region: 624-722aa. |

1 Associated with the cytoplasmic side of the cell membrane
L* stands for protein length in amino acids

TABLE 2

| Clone ID | Locus | Associated diseases |
|---|---|---|
| 1. Bcd0468 | 20q11.1-q11.22 | 1. Congenital dyserythropoietic anaemia type II (CDAN2) at 20q11.2. |
| 2. Bcu0021 | 16p13.3 | 1. Polycystic Kidney Disease (PKDTS) at 16.p13.3. |
| | | 2. Microphthalmia-cataract at 16p13.3. |
| 3. Bcu0067 | 5q14.1 | 1. Hyaloideoretinal degeneration of Wagner at5q13-q14. |
| 4. Bcu0120 | 3q25.1 | 1. Dandy-Walker variant malformation and hydrocephalus at 3q25.1. |
| 5. Bcu0148 | 6q21.3 | 1. Specific dystexia 2 at 6p21.3; |
| | | 2. Ankylosing spondylitis at 6p21.3; |
| | | 3. Renal glucosuria at 6p21.3; |
| | | 4. IDDM1 at 6p21.3; |
| | | 5. Atrial septal defect at 6p21.3; |
| | | 6. Hypotrichosis simplex of scalp at 6p21.3; |
| | | 7. Diffuse panbronchiolitis at 6p21.3; |
| | | 8. Immunoglobulin A deficiency susceptibility 1 at 6p21.3. |
| 6. Bcu0149 | 17q22-q23 | 1. Meckel syndrome, Type 1; MKS1 at 17q22-q23; |
| | | 1. Malignant Hyperthermia susceptibility 2; MHS2 at 17q11.2-q24; |
| 7. Bcu0092 | 7p14 | 1. Neuronal type D Charcot-Maine-Tooth Disease at 7p14. |
| 8. Bcu0156x | 9q34.3 | 1. Joubert syndrome 1 at 9q14.3; |
| | | 2. Recessive non-Friedreich spinocerebellar ataxia at 9q34; |
| | | 3. Primary autosomal recessive microcephaly 3 at 9q34; |
| | | 4. Juvenile amyotrophic lateral sclerosis 4 at 9q34; |
| | | 5. Lethal congenital contractures syndrome at 9q34. |
| 9. Bcu0258x | 2q32.3 | 1. Familial arrhythmogenic right ventricular dysplasia 4 at 2q32.1-q32.3; |
| | | 2. Wrinkly skin syndrome at 3q32. |
| 10. Bcu0343 | 10p11.1-p11.22 | 1. Thrombocytopenia at 10p11.2-p12. |
| 11. Bcu0371 | 19q13.2 | 1. Cystic fibrosis modifier 1 at 9q13.2-q13.4; |
| | | 2. OPA3 19q13.2-q13.3; |
| | | 3. Liposarcoma oncogene at 19q13.2-q13.3. |
| 12. Bcu0399 | 5q14.3 | 1. Usher syndrome type II at 5q14-q21; |
| | | 2. Hyaloideoretinal degeneration of wagner at 5q13-q14. |
| 13. Bcu0408 | 17q11.2 | 1. No disease association. |
| 14. Bcu0475z | 14q32.2 | 1. Usher syndrome type1A at 14q32; |
| | | 2. Autosomal recessive microphthalmos at 14q32; |
| | | 3. Hemifacial microsomia at 14q32; |
| | | 4. Ectopic expression of brain type of creatine kinase at 14q32. |
| 15. Bcu0504 | 9q32-q32.2 | 1. Muscular dystrophy, LIMB-Girdle type 2H at 9q31-q34.1. |
| 16. Bcu0571 | 8q24.12 | 1. Childhood absence epilepsy at 8q24; |
| | | 2. Benign adult familial myoclonic epilepsy at 8q24; |
| | | 3. Generalized idiopathic epilepsy at 8q24; |
| | | 4. Macular dystrophy-1, atypical vitelliform at 8q24; |
| | | 5. Epidermolysis bullosa simplex at 8q24; |
| | | 6. Langer-giedion syndrome at 8q24.11-q24.13; |
| | | 7. Spinocerebellar ataxia 16 at 8q22.1-q24.1. |
| 17. Bcu0720x | 4p14 | 1. Del(4)(14) cause breast and ovary cancers; |
| | | 2. Trisomy causes ovary and breast cancers; |
| | | 3. Monosomy causes breast, ovary and prostate tumors. |
| 18. Bcu0721z | 16q22.3 | 1. Aneurysmal bone cysts at 16q22; |
| | | 2. North American Indian childhood cirrhosis at 16q22; |
| | | 3. Acute myelogenous leukemia at 16q22. |
| 19. Bcu0730x | 15q21 | 1. Congenital dyserythropoietic anemia type III (CDAN3) at 15q21; |
| | | 2. Specific dystexia 1 at 15q21; |
| | | 3. Hereditary non-polyposis colorectal cancer type 7 at 15q21.1. |
| 20. Bcu0770 | 2q35 | 1. Brachydactyly, Type A1; BDA1 at 2q35-q36. |
| | | 2. Syndactyly, Type I at 2q34-q36. |
| 21. Bcu0840 | 12q24.1 | 1. Adult spinal muscular atrophy at 12q24; |
| | | 2. New England type neurogenic scapulopcroneal amyotrophy at |
| | | 12q24.1-q24.31. |
| 22. Bcu0862 | 17q12 | 1. Amplification of the c-erbB-2 locus (17q12-q21.32) causes gastric |
| | | cancer. |
| 23. Bcu0916z | 10q22.1 | 1. No genomic DNA available, therefore no data yet. |
| 24. Bcu918-2 | 4q21.3 | 1. Mucolipidosis II (I-cell disease) at 4q21-q23; |
| | | 2. Hyper-IgE Syndrome at 4q21; |
| | | 3. Mental health wellness 2 at 4p (Disease locus at D4S397). |
| 25. Bcu0947xz | 4p15.33 | 1. Autosomal dominant lewy body Parkinson disease 4 (PARK4) at |
| | | 4p15; |
| | | 2. Huntington disease-like 3 (HDL3) at 4p15.3; |
| | | 3. Susceptibility to systemic Lupus erythematosus 3 at 4p15.2-p16. |
| 26. Bcu1034 | 8q13.2 (Pr0453) | 1. Familial febrile convulsions 1; FEB1 at 8q13-q21. |
| 27. Bcu1041 | 17q21.1 | 1. Patella aplasia-hypoplasia at 17q21-q22; |
| | | 2. Familial progressive subcortical Gliosis at 17q21-q22; |
| | | 3. Pallidopontonigral degeneration at 17q21. |
| 28. Bcu0610Az | | 1. No genomic DNA available, therefore no data yet. |
| 28. Bcu0610Bz | | |
| 29. Bcu0586 | 16q13 | 1. Brooke-Spiegler syndrome at 16q12-q13. |
| 30. Bcu0715Az | 5q14.3 | 1. Hyaloideoretinal degeneration of Wagner at 5q13-q14; |
| | | 2. Trisomy and monosomy at 5q14 causes cancers in breast, ovary |
| | | and prostate. |
| 30. Bcu0715Bz | 5q14.3 | same as Bcu0715Az. |
| 30. Bcu0715Cz | 5q14.3 | same as Bcu0715Az. |
| 31. Bcu0205Az | 5q33.3 | 1. Atopic dermatitis 6 at 5q31-q33; |
| | | 2. Capillary infantile hemangioma at 5q31-q3; |
| | | 3. Asthma at 5q31-q33; |
| | | 4. Susceptibility/Resistance to Schistosoma mansoni infection at |
| | | 5q31-q33; |
| | | 5. Familial eosinophilia at 5q31-q33. |
| 31. Bcu0205Bz | 5q33.3 | same as Bcu0205Az |
| 32. Bcu0988 | 14q24.3 | 1. Leber congenital amaurosis type III at 14q24; |
| | | 2. Familial arrhythmogenic right ventricular dysplasia-1 (ARVD1) at |
| | | 14q23-q24. |
| 33. Bcu0518z | 10q24.32 | 1. Type II corneal dystrophy of bowman layer at 10q24; |
| | | 2. Infantile-onset spinocerebellar ataxia at 10q24. |
| 34. Bcu0147Az | 12q13.2 | 1. Autosomal dominant spastic paraplegia-10 at 12q13; |
| | | 2. Bothnian type palmoplantar keratoderma at 12q11-q13. |
| 34. Bcu0147z | 12q13.2 | same as Bcu0147z. |

TABLE 3

| Gene | | | |
|---|---|---|---|
| Name | SEQ ID NO | Expression | |
| BCD0468 | 1-2 (A); 3-4 (B); | DOWN | |
| | 5-6 (C) | | |
| BCU0021 | 7-8 | UP IL | |
| BCU0067 | 9-10 | UP IL | |
| BCU0120 | 11-14 | UP IL | normal expression |
| | | | restricted to breast |
| BCU0148 | 15-16 | UP IL | |
| BCU0149 | 17-18 | UP DIH | |
| BCU0092 | 19-20 | UP DL | |
| BCU0156 | 21-22 | UP DIL | normal expressed |
| | | | restricted to thymus |
| BCU0258 | 23-24 | UP DIL | |
| BCU0343 | 25-26 | UP DIM | |
| BCU0371 | 27-28 (A); 29-30 (B) | | |
| BCU0408 | 31-32 | UP DIL | |
| BCU0475 | 33-34 | UP DH | |
| BCU0504 | 35-36 | UP DIL | |
| BCU0571 | 37-38 | UP DIL | |
| BCU0720 | 39-40 | UP DIM | |
| BCU0721 | 41-42 | UP IL | |
| BCU0730 | 43-44 (A); 45-46 (B); | UP DIL | |
| | 47-48 (C); 49-50 (D) | | |
| BCU0770 | 51-52 | IH | |
| BCU0840 | 53-54 | UP DH | normal expression |
| | | | restricted to adrenal |
| | | | and prostate glands |
| BCU0862 | 55-56 | UP DH | |
| BCU0916 | 57-58 | UP DL | |
| BCU0918 | 59-60 | UP DM | |
| BCU0947 | 61-62 | UP DIL | |
| BCU1034 | 63-64 | UP DL | |
| BCU1041 | 65-66 | UP DL | |
| BCU0610 | 67-68(A); 69-70(B) | UP DIH | |
| BCU0586 | 71-72 | UP DIM | |
| BCU0715 | 73-74 (A); 75-76 (B); | UP DIM | |
| | 77-78 (C) | | |
| BCU0205 | 79-80 (A); 81-82 (B) | UP DL | |
| BCU0988 | 83-84 (A); 85-86 (B) | UP DIM | |
| BCU0518 | 87-88 | UP DIL | |
| BCU0147 | 89-90 (A); 91-92 (B) | UP IL | |

I = invasive ductal carcinoma (IDC);
D = ductal carcinoma in situ (DCIS);
L = low expression;
M = medium expression;
H = high expression

TABLE 4

| | | | |
|---|---|---|---|
| | | | Predominant Sites |
| Gene Name and SEQ ID | Regulation | Description | of Expression |

| | | | | |
|---|---|---|---|---|
| Bcu41 | (SEQ ID NO 115, 116) | Up | transmembrane Zn protease | |
| Bcu656 | (SEQ ID NO 117) | Up | noncoding RNA | |
| Bcu738 | (SEQ ID NO 118-119) | Up | ribosomal protein | |
| Bcu93 | (SEQ ID NO 120-121) | Up | ribosomal protein L23 | |
| Bcu931 | (SEQ ID NO 123-124) | Up | Ras-related GTP-binding protein | |
| Bcd215 | (SEQ ID NO 125-126) | Down | carboxypeptidase | |
| Bcu921 | (SEQ ID NO 127-128) | Up | | |
| Bcu224 | (SEQ ED NO 129-130) | Up | transmembrane BMP receptor | |
| Bcu259 | (SEQ ID NO 131-132) | Up | nuclear localizing protein | |
| Bcu616 | (SEQ ID NO 133-134) | Up | | |
| Bcu631 | (SEQ ID NO 135-136) | Up | | |
| Bcu1040 | (SEQ ID NO 137-138) | Up | | |
| Bcu924 | (SEQ ID NO 139-140) | Up | | |
| BCD0332 | (SEQ ID NO 141-142) | Up | slow voltage-gated potassium channel, 2-123 | |
| | | transmembrane domain, 47-69 | | |
| BCU0627 | (SEQ ED NO 143-144) | Up | signal peptide, 1-20 | colon, muscle |
| | | transmembrane domain, 21-40 | | |
| | | peptidase family M13, 538-748 | | |
| BCD0269 | (SEQ ID NO 145-146) | Up | transmembrane domain, 45-67 | adrenal, heart, |
| | | transmembrane domain, 80-102 | lung | |
| BCU0724 | (SEQ ID NO 147-148) | Up | transmembrane domain, 62-84 | pancreas, heart, |
| | | transmembrane domain, 91-108 | liver, breast, | |
| | | transmembrane domain, 113-135 | pancreas | |

1. An isolated polynucleotide comprising a polynucleotide sequence which codes without interruption for a human differentially-regulated breast cancer polynucleotide having an amino acid sequence set forth in SEQ ID NOS 2, 4, 6, 8, 10, 12, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 116, 119, 121, 124, 126, 128, 130, 132, 134, 136, 138, 140, 142, 144, 146, or 148, or a complement thereto.

2. An isolated polynucleotide of claim 1 having a polynucleotide sequence set forth in selected from SEQ ID NOS 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, or 147.

3. An isolated polynucleotide comprising, a polynucleotide coding sequence for a human differentially-regulated breast cancer gene having 95% or more nucleotide sequence identity along its entire length to a polynucleotide sequence set forth in SEQ ID NO 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, or 147, and which codes without interruption for said coding sequence, or a complement thereto.

4. An isolated polynucleotide which is specific for a human differentially-regulated breast cancer gene of claim 1 and having a nucleotide sequence selected from: SEQ ID NO 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 115, 117, 118, 120, 123, 125, 127, 129, 131, 133, 135, 137, 139, 141, 143, 145, or 147, or a complement thereto.

5. An isolated polynucleotide of claim 4, wherein said fragment is effective in a polymerase chain reaction.

6. An isolated polypeptide of claim 1 which is differentially-regulated in breast cancer having an amino acid sequence set forth in SEQ ID NOS 2, 4, 6, 8, 10, 12, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 116, 119, 121, 124, 126, 128, 130, 132, 134, 136, 138, 140, 142, 144, 146, or 148.

7. An isolated polypeptide which is a human differentially-regulated breast cancer gene having 95% or more amino acid sequence identity along its entire length to an amino acid sequence selected from SEQ ID NOS 2, 4, 6, 8, 10, 12, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 116, 119, 121, 124, 126, 128, 130, 132, 134, 136, 138, 140, 142, 144, 146, or 148.

8. A method of detecting a nucleic acid for a human differentially-regulated breast cancer gene, comprising,
contacting a sample comprising nucleic acid with a polynucleotide probe specific for a human differentially-regulated breast cancer gene of claim 1 under conditions effective for said probe to hybridize specifically with a nucleic acid for said gene, and
detecting hybridization between said probe and said nucleic acid.

9. A method of claim 8, wherein said detecting is performed by:
Northern blot analysis, polymerase chain reaction (PCR), reverse transcriptase PCR, RACE PCR, or in situ hybridization.

10. A method of treating a breast cancer showing elevated expression of a human differentially-regulated breast cancer gene, comprising:
administering to a subject in need thereof a therapeutic agent which is effective for regulating expression of a human differentially-regulated breast cancer gene polynucleotide, or polypeptide encoded thereby, of claim 1.

11. A method for identifying an agent that modulates the expression of a human differentially-regulated breast cancer gene in cells expressing said gene, or the biological activity of a polypeptide encoded thereby, comprising,
contacting a cell with a test agent under conditions effective for said test agent to modulate the expression of a human gene of claim 2, or the biological activity of a polypeptide encoded thereby, in said cell, and
determining whether said test agent modulates said gene or polypeptide.

12. A method of claim 11, wherein said agent is a binding-partner that specifically recognizes said polypeptide.

13. A method of detecting polymorphisms in a human differentially-regulated breast cancer gene, comprising,
comparing the structure of: genomic DNA comprising all or part of a human differentially-regulated breast cancer gene, mRNA comprising all or part of a human differentially-regulated breast cancer gene, cDNA comprising all or part of a human differentially-regulated breast cancer gene, or a polypeptide comprising all or part of a human differentially-regulated breast cancer gene, with the complete structure of a human differentially-regulated breast cancer gene of claim 2.

14. A method of claim 13, wherein said polymorphism is a nucleotide deletion, substitution, inversion, or transposition.

15. A mammalian cell whose genome comprises a functional disruption of a differentially-regulated breast cancer gene homolog of claim 1 within a nucleotide sequence which is specific for said gene.

16. A non-human, transgenic mammal comprising a cell of claim 15, said mammal being susceptible to breast cancer.

17. An antibody which is specific-for a polypeptide sequence which is specific for a human differentially-regulated breast cancer gene of claim 1.

18. A method of selecting a human differentially-regulated breast cancer gene polynucleotide or amino acid sequence from a database, comprising:
displaying, in a computer-readable medium, a polynucleotide sequence or polypeptide sequence for a human differentially-regulated breast cancer gene of claim 2, or complements to the polynucleotides sequence,
wherein said displayed sequences have been retrieved from said database upon selection by a user.
